(19) United States
(12) Patent Application Publication
(10) Pub. No.: US 2013/0149336 A1
(43) Pub. Date: Jun. 13, 2013

(54) Methods for Screening Viral Like Particles and Identifying Neutralizing Epitopes and Related Vaccines, Constructs, and Libraries

(71) Applicants: Brian L. Hjelle, Placitas, NM (US); David S. Peabody, Albuquerque, NM (US); Bryce Chackerian, Albuquerque, NM (US)

(72) Inventors: Brian L. Hjelle, Placitas, NM (US); David S. Peabody, Albuquerque, NM (US); Bryce Chackerian, Albuquerque, NM (US)

(21) Appl. No.: 13/662,060

(22) Filed: Oct. 26, 2012

Related U.S. Application Data
(60) Provisional application No. 61/622,154, filed on Apr. 10, 2012.
(60) Provisional application No. 61/552,008, filed on Oct. 27, 2011.

(51) Int. Cl. G01N 33/68

(52) U.S. Cl. CPC G01N 33/6878
USPC 424/204.1; 506/2; 435/5; 536/23.72

(57) ABSTRACT

The invention is directed to methods of screening immunogenic viral like particles and related immunogenic compositions and diagnostic techniques. In one embodiment, the invention provides methods of screening immunogenic viral like particles containing peptides corresponding to epitope regions of a wide variety of pathogens, including viruses, bacteria, parasites, and microbes. Non-infectious antigens and allergens of interest can also be screened as described herein. Immunization, therapeutic and diagnostic applications are also described for the compositions and methods according to the invention.

In another embodiment, the invention provides novel methods of identifying a cryptic neutralizing epitope and related vaccines, constructs, and libraries. In some embodiments, these methods use high-throughput formats that are facilitated by in silica or in vitro steps.

### RELATED APPLICATIONS

This application claims priority from U.S. Provisional Application No. 61/622,154, filed on Apr. 10, 2012, entitled “A Method of Screening Viral Like Particles for Utility in Immunodiagnostics, Allergic Diseases and Vaccine Development”, and U.S. Provisional Application No. 61/552,008, filed on Oct. 27, 2011, entitled “Methods for the Identification of Neutralizing Epitopes and Related Vaccines, Constructs, and Libraries”. The complete disclosures of these applications are hereby incorporated by reference in their entireties herein.

### GOVERNMENT INTEREST

This patent application was supported by National Institute of Allergy and Infectious Diseases Grant No. 1R01AI083305 and National Institute of General Medical Sciences Grant No. R01 GM042901. The Government has certain rights in the invention.

### FIELD OF THE INVENTION

The invention provides methods of screening immunogenic viral like particles and related immunogenic compositions and diagnostic techniques and kits.

In one embodiment, the invention provides methods of screening immunogenic viral like particles containing peptides corresponding to epitope regions of a wide variety of pathogens, including viruses, bacteria, parasites, and microbes. Non-infectious antigens and allergens of interest can also be screened.

In another embodiment, the invention provides novel methods of identifying a cryptic neutralizing epitope and related vaccines, constructs, and libraries. In some embodiments, these methods use high-throughput formats that are facilitated by in silica or in vitro steps.

The invention makes cryptic neutralizing epitopes potently immunogenic and provides a wide variety of potential vaccine candidates. In certain embodiments, methods of the invention are useful in the identification of cryptic neutralizing epitopes ranging in size from a few hundred to several thousands of amino acids.

### BACKGROUND OF THE INVENTION

Essential to the development of any vaccine is the identification of an immunogen that provokes a protective immune response. The specificity of the natural antibody response to infection can itself be used as a guide to the identity of relevant antigens, but the interaction of pathogens with the immune system is a complex arms race of mutual adaptation, and some infectious agents have learned to chronically or repeatedly infect their hosts by exhibiting lots of antigenic variation in the sequences of neutralizing epitopes, while hiding invariant ones. In such cases knowing the identities of immunodominant epitopes is often not very useful, either because they are being constantly changed by mutation and immune selection, or because they are non-neutralizing in the first place.

There are several cases where cryptic epitopes can elicit neutralizing antibodies if they could only somehow be made immunogenic. A search for candidate vaccine epitopes would benefit from the construction of a detailed epitope map for the pathogen in question, and an assessment of the ability of each epitope to elicit a neutralizing antibody response when made sufficiently immunogenic.

It is often assumed that a vaccine should target multiple epitopes, but in fact a large fraction of the antibodies elicited by a given pathogen or vaccine may be irrelevant in protective immunity (e.g. because they are non-neutralizing), or even harmful (e.g. by mediating antibody dependent enhancement of infection). A more intelligent approach would first produce a thorough map of epitopes, distinguishing those that elicit neutralizing antibodies from those that don't, and perhaps especially targeting cryptic neutralizing epitopes. (Non-neutralizing epitopes might find other uses, such as in diagnostics.) Peptide-based vaccines could then be generated to elicit highly specific and potent neutralizing antibody responses. If incorporation of multiple epitopes into a vaccine proves to be important, they can be presented as mixtures that elicit antibody responses only to the most efficacious epitopes.

Vaccines based on peptides alone are generally poorly immunogenic, particularly in humans. Immunogenicity of peptide antigens can be increased dramatically by displaying them in a multivalent format on the surface of virus-like particles (VLPs), where they induce strong antibody responses at low doses and without exogenous adjuvants. VLP-based vaccines have favorable safety profiles and have already been shown to be effective in several human clinical trials, indicating that application of this technology has the potential to dramatically accelerate the development of new human vaccines.

A major hurdle in VLP vaccine development is the difficulty in both identifying relevant target epitopes and then presenting them to the immune system in a highly immunogenic fashion. Conventional phage display mostly uses filamentous phages like M13, and while it is a powerful system for epitope mapping, it suffers from the drawback of limited immunogenicity of the peptides it displays. This is the consequence of a quirk of filamentous phage molecular biology that makes it difficult to display foreign peptides are the high densities that best favor high immunogenicity. To test a peptide's value as a potential epitope vaccine, it is usually necessary first to synthesize it chemically and then conjugate it to a more immunogenic carrier—an expensive, time consuming and inconvenient process. Even worse, some epitopes lose affinity for the selecting target (and ability to elicit the desired antibody response) when moved from the structural environment they experienced during affinity selection. We have developed a new peptide display and affinity selection platform that integrates the epitope discovery and immunization functions into a single virus-like particle derived from RNA bacteriophage MS2. The system has already demonstrated its utility for finding linear epitopes, and, in now showing the ability to find peptide mimics of protein conformational epitopes.

The recent discovery of a new class of broadly neutralizing antibodies for viruses like HIV and influenza A has raised anew the old hopes for universal vaccines against these deadly pathogens. It is now appreciated that such antibodies are frequently produced by infected individuals, but in such low concentrations that they are largely ineffective in controlling infection (except perhaps in so-called elite neutralizers). In the case of HIV, for example, new broadly neutralizing antibodies have been found by searching the B-cell population of infected persons for those rare cells that produce antibodies with the desired properties. These antibodies appear to recognize epitopes that are poorly immunogenic, but should nevertheless be able to elicit a neutralizing response when rendered sufficiently immunogenic. Apparently the virus has evolved to bury these sequence-invariant epitopes, hiding them from the immune system, while exposing other epitopes whose sequences are more tolerant of mutation. The occasional transient exposure of such epitopes, whether by thermal fluctuations in virus structure or as a consequence of conformational changes accompanying cell entry, makes them targets for neutralization whenever the antibodies that bind them are present at high enough concentration. We refer to these as cryptic neutralizing epitopes, and think they are likely to be more common than we have appreciated, especially in pathogens that chronically or repeatedly infect their hosts.

Protective antigen-based vaccines may elicit a limited neutralizing antibody response. For example, consider the now familiar case of a vaccine based on the Human Papilloma Virus L2 protein. Existing vaccines now in clinical use are based on the major capsid protein of HPV, called L1, and are limited in their effectiveness by the diversity of L1 sequences encountered in different HPV strains. The L2 protein sequence is far less variable. We now understand that antibodies against L2 are neutralizing, even though anti-L2 antibodies are not normally elicited in significant amounts during HPV infection. In other words, L2 epitopes are neutralizing but non-immunogenic. This is probably because they are only transiently exposed during the infection process, and have little opportunity to elicit an immune response. However, during infection their transient exposure makes them susceptible to binding (and neutralization) by any pre-existing antibodies elicited by an L2-based vaccine. In other words, the relevant L2 peptides may be said to contain cryptic neutralizing epitopes. Existing methods of vaccine discovery generally rely on knowledge of neutralizing epitopes elicited normally by exposure of a subject to an antigen. By their nature cryptic neutralizing epitopes are not detectable by such methods.

Other examples of cryptic neutralizing epitopes have been discovered in HIV infections. HIV evades the immune system by varying the sequences of its most prominent epitopes, so although infected individuals produce neutralizing antibodies, the dominant epitopes keep changing. Rare individuals, however, somehow manage to produce antibodies with neutralizing activity for diverse HIV strains by producing antibodies that bind epitopes that are normally cryptic. The sequences of these peptides are not as subject to variation as the dominant epitopes and are therefore largely conserved in otherwise widely divergent HIV strains, providing hope that a vaccine could be developed that targets such cryptic neutralizing epitopes.

In another example, current evidence suggests that protective antigen (PA)-based anthrax vaccines may elicit a narrow neutralizing antibody repertoire, and this may represent a vulnerability with PA-based vaccines. Oscherwitz, et al., Synthetic Peptide Vaccine Targeting a Cryptic Neutralizing Epitope in Domain 2 of Bacillus anthracis Protective Antigen, Infection and Immunity, August 2009, p. 3380-3388, Vol. 77, No. 8

The aforementioned limited neutralizing antibody response common to a variety of pathogens creates a need for useful antigen screening techniques that identify cryptic neutralizing epitopes and facilitate related vaccine production. In other words, methods are needed that can identify peptides that would be capable of eliciting a neutralizing antibody response, if only they could be rendered immunogenic. We envision a method that creates libraries of peptides derived from the sequences of potential antigens on virus-like particles (VLPs), and then tests large numbers of individual members of the library for the ability to elicit a neutralizing antibody response in an appropriate model organism. Optimally, libraries of fragments of potential antigens would be provided on virus-like particles (VLP's) and individual members of the libraries would be screened directly for their ability to elicit a neutralizing antibody response. Technical limitations have prevented the application of such approaches until now. Current challenges include difficulty in efficiently synthesizing a sufficiently complex antigen fragment library. Also, it has proven difficult to screen large numbers of VLP's for those few having an ability to elicit a desired antibody response. Further, there is a need for techniques which enable both purification of a large number of immunogenic antigens, and assaying antigens for a desired response in a high-throughput manner.

The need also exists for methods that seek to identify relevant epitopes directly by affinity selection, and that also subsequently screen for an ability to elicit a neutralizing response. Preferably, such methods would enable affinity selection on a platform that, because of its intrinsically high immunogenicity, allows selected peptides to be directly tested for their ability to elicit neutralizing antibodies.

Making an epitope “fingerprint” using the antibodies elicited after exposure of a human or animal to an infectious agent or other antigen would also prove useful for applications other than vaccine development, e.g. diagnostic applications, since VLPs displaying pathogen-specific, antigen-specific, or allergen-specific epitopes could be used to detect the presence of a specific antibody response.

The invention described here has the potential to create a comprehensive “epitope profile” that characterizes the totality of an individuals antibody response, thus enabling the identification of rare, relatively non-immunogenic epitopes.

### SUMMARY OF THE INVENTION

Exposure to an antigen (e.g. during infection by a pathogen, exposure to an allergen, or an epitope which elicits an autoantibody response, etc.) results in the production of antibodies that recognize specific epitopes on the antigen. The method described here allows the identification of the spectrum of epitopes recognized by antibodies produced in response to antigen exposure. The basic idea is to create peptide libraries on the surface of RNA phage VLPs and then utilize antibodies found in the sera of infected individuals or monoclonal antibodies derived from infected or immunized human or animal hosts to affinity-select the epitopes they recognize from the library. The library would normally be one of two types: 1. Antigen fragment libraries are constructed using sequences derived from the known primary structure of an antigen. This kind of library contains only sequences found in the antigen(s) of interest. 2. Random sequence libraries are produced by display of randomly generated peptide sequences on the VLP surface. In both cases affinity selection with appropriate antisera selectively isolates and amplifies VLPs bearing epitopes bound by the antibodies. The resulting selected VLPs can be characterized by a variety of methods, but the use of deep sequence analysis allows the identification of virtually all the peptides recognized by the antibody population. These VLPs can then be utilized as immunogens (i.e. vaccines) or as reagents to detect the presence of specific antibody responses in, for example, diagnostic assays, thus providing evidence of exposure to the antigen.

The methods described herein generate reagents that can document a subject's exposure to a wide variety of different agents. Affinity selected VLPs, and the epitopes they carry are utilized in a variety of formats for the detection of antibodies characteristically present in the sera of individuals exposed to an agent of interest e.g. Dengue Virus infection or an allergen, or to an epitope which produces an autoantibody response or a response to a foreign antigen or an allergenic protein or carbohydrate. In one embodiment, the invention enables the production of reagents that not only verify the type of infecting agent (e.g. Dengue Virus), but that also distinguish an infectious agent serotype.

In one embodiment, the invention provides a method of screening immunogenic viral like particles comprising:

(a) a library of peptides which have been expressed on virus-like particles comprising a preferably a MS2 single chain coat polypeptide dimer or a bacteriophage PP7 single chain coat polypeptide dimer, each of said peptides corresponding to a putative epitope, including a putative epitope of a pathogen, an allergen or an epitope which produces an autoantibody response or a response to a foreign antigen or an allogenic protein, said virus-like particles having been made by prokaryotically expressing nucleic acid constructs which each comprise an oligonucleotide encoding one of the peptides corresponding to the putative epitope of the pathogen;
(b) conducting affinity selection on the library of virus-like particles using monoclonal antibodies or antiserum, including anti-pathogen or anti-allergen antiserum (among others), to select those virus-like particles that bind to the antibodies of the antiserum;
(c) conducting affinity selection using a specific subset or class/subclass of immunoglobulins that are present in serum, plasma or bodily fluid, including IgM antibodies (such as for development of serodiagnostics for acute infectious diseases) or IgE antibodies (such as for development of diagnostics or vaccines for allergic conditions)
(d) sequencing the cDNAs encoding peptides which have been expressed on the candidate virus-like particles;
(e) prokaryotically expressing the sequenced peptides on virus-like particles comprising a bacteriophage MS2 single chain coat polypeptide dimer or a bacteriophage PP7 single chain coat polypeptide dimer and thereafter purifying the virus-like particles comprising the sequenced peptides; and optionally
(f) immunizing an animal or human subject with the purified virus-like particles comprising the sequenced peptides and assaying the subject's immune response upon exposure to the source of epitope, including a pathogen, allergen or other source of epitope.

In certain embodiments, the source of the epitope is a pathogen such as a virus, a bacterium, a parasite, or a microbe. For example, the pathogen can be selected from the group consisting of dengue virus; yellow fever virus; West Nile virus; Japanese encephalitis virus; HIV; HTLV-I, Bunyaviridae viruses including the hantaviruses, Crimean-Congo hemorrhagic fever, Rift Valley fever virus, and severe fever and thrombocytopenia virus; arenaviruses including all agents of South American hemorrhagic fever, Lassa virus and lymphocytic choriomeningitis virus; filoviruses including Ebola and Marburg viruses; paramyxoviruses including morbilliviruses, henipaviruses, respiroviruses including RSV and metapneumovirus and rubellaviruses; Alphaviruses including Chikungunya, O'nyung-nyung, Semliki Forest, Ross River, Sindbis, eastern, western and Venezuelan equine encephalitis; picornaviruses; papillomaviruses including HPV; herpesviruses including HSV-1/2, EBV, CMV, HHV-6, 7, and 8; polyomaviruses including SV40, JC and BK viruses; poxviruses including variola and vaccinia viruses; bacterial pathogens including any human pathogen such as Staphylococcus spp; Streptococcus spp; Burkholderia, mycoplasma, E. coli, and other pathogenic coliforms; and parasitic pathogens including malaria (Plasmodium spp).

In other embodiments, the source of the epitope is an allergen or source which produces an autoantibody response or a response to a foreign protein or carbohydrate antigen, an autoimmune antigen, an allogeneic protein or allergen.

The library of peptides which have been expressed on virus-like particles is either an antigen fragment library or a random sequence library.

Methods for peptide display on the VLPs of RNA bacteriophages, including MS2 and PP7, have been disclosed previously and are described in detail hereinafter.

In certain embodiments of the screening methods, peptides which have been expressed on selected virus-like particles are sequenced using deep sequencing.

In another embodiment, (a) the library of peptides which have been expressed on virus-like particles comprising a bacteriophage MS2 single chain coat polypeptide dimer or a bacteriophage PP7 single chain coat polypeptide dimer is a random sequence library; and (b) the pathogen is a virion containing a glycoprotein that maps to a sequenced peptide of a purified virus-like particle.

In still another embodiment, (a) the library of peptides which have been expressed on virus-like particles comprising a bacteriophage MS2 single chain coat polypeptide dimer or a bacteriophage PP7 single chain coat polypeptide dimer is a random sequence library; and (b) one or more members of the random sequence library is recognized by antibodies against the pathogen, either as a linear or conformational epitope.

In a particular embodiment, (a) the library of peptides which have been expressed on virus-like particles comprising a bacteriophage MS2 single chain coat polypeptide dimer or a bacteriophage PP7 single chain coat polypeptide dimer is an antigen fragment library; and (b) peptides which have been expressed on virus-like particles in the antigen fragment library have been prokaryotically expressed from oligonucleotides which in the aggregate scan the pathogen's genome. Preferably, screening methods of the invention are conducted in a high-throughput format.

In certain embodiments, the pathogen has more than one serotype and the peptides corresponding to a putative epitope of the pathogen, include peptides corresponding to epitopes of each pathogen serotype. Further, the pathogen antiserum can be a polyclonal antiserum.

Thus, in one embodiment of a screening method of the invention:

(a) affinity selection on the library of virus-like particles using pathogen antiserum comprises comparing an affinity range for a variety of neutralizing antibody titers;
(b) the pathogen has more than one serotype and the peptides corresponding to a putative epitope of the pathogen include peptides corresponding to epitopes of each pathogen serotype; and
(c) the pathogen antiserum is a polyclonal antiserum.

Preferably, peptides are expressed on virus-like particles comprising a bacteriophage MS2 single chain coat polypeptide dimer.

In certain embodiments, the step of prokaryotically expressing the sequenced peptides on virus-like particles comprising a bacteriophage MS2 single chain coat polypeptide dimer or a bacteriophage PP7 single chain coat polypeptide dimer comprises:

(a) selecting those phage that bind to antibodies (IgG, IgM, IgA, IgE or IgY) with desired anti-pathogen, anti-allergen, or anti-antigen specificity, and preparing cDNA and/or amplifying by PCR to determine the sequence of the phage that have been selected for binding to said antibodies; and (b) constructing a library of virus-like particles by (1) providing a plurality of nucleic acid constructs, treating the nucleic acid constructs with a restriction enzyme, and inserting the one or more nucleic acid sequences ascertained in step (a) into the nucleic acid constructs to obtain a population of transcription units (2) generating virus-like particles by expressing the transcription units in a prokaryote which has been modified to under-express an affinity tag, and (3.) purifying the viral-like particles using the affinity tag and isolating the library.

In still other embodiments, peptides corresponding to a putative epitope of a pathogen comprise a series of peptide mer units comprising between about 4 to about 100. about 5 to about 100 amino acids, between about 5 to about 90 amino acids, between about 5 to about 80 amino acids, between about 5 to about 70 amino acids, between about 5 to about 60 amino acids, between about 5 to about 50 amino acids, between about 5 to about 40 amino acids, often between about 5 to about 30 amino acids, more often between about 5 to about 20 amino acids, and often between about 2 to about 10 amino acids, or about 4 to about 8 amino acids.

Preferably, the nucleic acid constructs used to express putative epitope peptide-containing viral like particles comprise: (a) a bacterial or bacteriophage promoter which is operably associated with a coding sequence of either bacteriophage MS2 single chain coat polypeptide dimer or bacteriophage PP7 single chain coat polypeptide dimer, wherein the coat polypeptide dimer coding sequence (1) is modified to define a first restriction site positioned 5′ to that portion of the sequence which defines the coat polypeptide dimer AB loop, (b) a restriction site positioned 3′ to the coat polypeptide dimer coding sequence; (c) a PCR primer positioned 3′ to the second restriction site; (d) a gene for resistance to a first antibiotic; (e) a helper phage gene modified to contain a gene conferring resistance to a second antibiotic, and (f) a replication origin for replication in a prokaryotic cell.

Preferably, a first primer is positioned 5′ to the first restriction site and a second primer is positioned 3′ to the second restriction site and 5′ to the PCR primer. In some embodiments, the bacterial or bacteriophage promoter is a T7 promoter, the first restriction site is either a SalI and KpnI restriction site, the second restriction site is a BamHI site, the PCR primer is TP7, the antibiotic repressor is a kanamycin resistance gene, and the replication origin is colE1 ori. The construct can also comprise a transcription terminator positioned 5′ to the second restriction site and optionally contains a transcription terminator positioned 5′ to the second restriction site.

In one embodiment of the nucleic acid constructs used to express putative epitope peptide-containing viral like particles, the bacterial or bacteriophage promoter is a T7 promoter, the RNA bacteriophage single chain coat polypeptide dimer is a MS2 coat protein single chain dimer, the codon sequence contains the maximum possible number of silent nucleotide substitutions relative to wild type coat protein, the restriction site is a BamHI site, the PCR primer is TP7, the repressor to resistance to a first antibiotic is a kanamycin resistance gene, the helper phage gene is modified to contain a gene conferring resistance to chloramphenicol, and the replication origin is colE1 ori.

In preferred viral like particle screening methods of the invention, peptides corresponding to a putative epitope of a pathogen in step (a) and sequenced peptides in step (e) are displayed on virus-like particles and encapsidate either MS2 mRNA or PP7 mRNA.

In other embodiments, the invention provides an immunogenic composition comprising a population of purified virus-like particles that have been identified by a screening method of the invention as described herein.

In still other embodiments, the invention provides a method of characterizing an immune response of a sample by contacting the sample with purified virus-like particles that have been identified by a screening method of the invention as described herein and assaying any resultant immune response. Such a sample can be obtained from a mammal that may be infected with a virus, a bacterium, a parasite, or a microbe, or alternatively, an allergen or source which produces an autoantibody response or a response to a foreign antigen or an allogeneic protein or carbohydrate antigen.

In one embodiment, the invention provides a method of identifying a cryptic neutralizing epitope comprising:

(a) defining one or more mimetics of potential viral cryptic neutralizing epitope-targeting peptide sequences by selection and immunization with the corresponding VLP to demonstrate their ability to neutralize pathogen, allergen or antigen;
(b) determining peptide sequences for each of the defined potential viral cryptic neutralizing epitope-targeting peptide sequences by;
(c) using RT-PCR and sequencing to determine the nucleic acid sequences which encode potential viral cryptic neutralizing epitope-targeting peptide sequences;
(d) constructing specific virus-like particles by (i) having the insert DNAs synthesized in vitro and treating the nucleic acid constructs with a restriction enzyme, and inserting one or more nucleic acid sequences ascertained in step (c) into the nucleic acid constructs to obtain a population of transcription units (ii) generating virus-like particles by expressing the transcription units in a prokaryote which has been modified to under-express an affinity tag, and (iii) purifying the viral-like particles using the affinity tag and isolating the library;
(e) immunizing a subject with one or more of the viral-like particles; and
(f) identifying one or more viral-like particles that induce a neutralizing antibody response in the subject.

The potential viral cryptic neutralizing epitope-targeting peptide sequences can target a wide variety of peptides, including an HIV peptide, a self antigen, amino acid sequences derived from the minor capsid protein L2 of human Papillomavirus type 16 (HPV16), the V3 loop of HIV-1 gp120, and Bacillus anthracia protective antigen, or any of the pathogens previously mentioned as well as allergens.

Methods of the invention can be performed in a high-throughput format, and various steps (e.g. immunizing and identifying) can be performed in vivo, in vitro, or in silica.

Because of their versatility, methods of the invention can generate virus-like particles comprising a series of peptide AB loop or N-terminal inserts comprising between about 5 to about 100 amino acids, more preferably between about 5 to about 50 amino acids, even more preferably between about 5 to about 10 amino acids. These series can overlap peptide regions adjacent to potential cryptic neutralizing epitope-targeting peptide sequences by between about 2 to about 100 amino acids, more preferably by between about 5 to about 50 amino acids, and even more preferably between about 5 to about 10 amino acids.

As further evidence of their wide applicability, in methods of the invention, the sequences of the viral-like particles can be known prior to the identification of one or more viral-like particles that induce a neutralizing antibody response in the subject, or the sequences of the viral-like particles can remain unknown until after to the identification of one or more viral-like particles that induce a neutralizing antibody response in the subject.

In a preferred embodiment (see for example pDSP62, FIG. 2X), a transcription unit used in methods of the invention comprises:

(a) a bacterial or bacteriophage promoter which is operably associated with a coding sequence of either bacteriophage MS2 single chain coat polypeptide dimer or bacteriophage PP7 single chain coat polypeptide dimer, wherein the coat polypeptide dimer coding sequence (1) is modified to define a first restriction site positioned 5′ to that portion of the sequence which defines the coat polypeptide dimer AB loop, and (2) comprises a nucleic acid sequence encoding one of the potential cryptic neutralizing epitope-targeting peptide sequences;
(b) a second restriction site positioned 3′ to the coat polypeptide dimer coding sequence; (c) a PCR primer positioned 3′ to the second restriction site;
(d) an antibiotic resistance gene which is operably associated with the promoter, and
(e) a replication origin for replication in a prokaryotic, and
(f) a replication origin from a single-strand DNA phage such as M13

Beyond the methods and constructs identified above, the invention also provides prokaryotes transformed by the nucleic acid constructs described herein, methods for constructing a library of such virus-like particles, methods for identifying peptides and for isolating an immunogenic protein, and immunogenic compositions.

These and other aspects of the invention are described further in the Detailed Description of the Invention.

### BRIEF DESCRIPTION OF THE FIGURES

FIG. 1. A: VLPs of the RNA phage MS2. The coat protein dimer edge-on with its AB-loops (lighter) in and its two polypeptide chains in red and green (darker). B: The VLP with the AB-loops (lighter). C: The dimer as seen from outside the VLP. The figure shows the proximity of N- and C-termini of the two polypeptides. Genetic fusion of these termini produce the single-chain dimer.

FIG. 2. A plasmid structure illustrating pDSP62 and pDSP7, the vectors used for library construction on MS2 and PP7 VLPs. Each plasmid differs only in the identity of the coat protein it expresses, pDSP62 being specific for MS2, and pDSP7 being specific for PP7 coat protein expression. The plasmids are otherwise identical.

FIG. 3. Nipah Virus G protein epitopes of five different mAbs mapped by affinity selection using MS2VLPs. The sequence of each selectant is shown, with perfect matches (in bold underline) to the relevant portions of the natural sequence (G) shown below it. mAB101 and 102 identify a series of overlapping peptides that apparently correspond to two overlapping epitopes. This figure shows the ability of the VLP affinity selection method to identify linear epitopes of neutralizing antibodies for an important pathogen.

FIG. 4. (left) The ED3 domain of the envelope protein of Dengue Virus. In red are shown amino acid residues whose substitution results in the inhibition of binding of several neutralizing monoclonal antibodies, illustrating the discontinuous, conformational nature of the epitope. (right) Affinity selection with one of these antibodies (MDVP-55A) resulted in selection of the peptide whose sequence is shown. Reaction with Dengue envelope protein (by ELISA) of antisera elicited in three mice by immunization either with MS2VLPs, or with MS2 VLPs displaying the clone 9 MDVP55A selectant (three mice) is also shown. These results show that the VLP platform is able to discover immunogenic mimics of discontinuous protein epitopes.

FIG. 5. Schematic representation of a screening method of the invention.

FIG. 6. An example of a high-throughput screening assay for virus neutralization. This specific case shows a flow cytometry assay of neutralization of a Sin Nombre Virus-based pseudovirus expressing GFP. Inhibition of luminescence corresponds to virus neutralization.

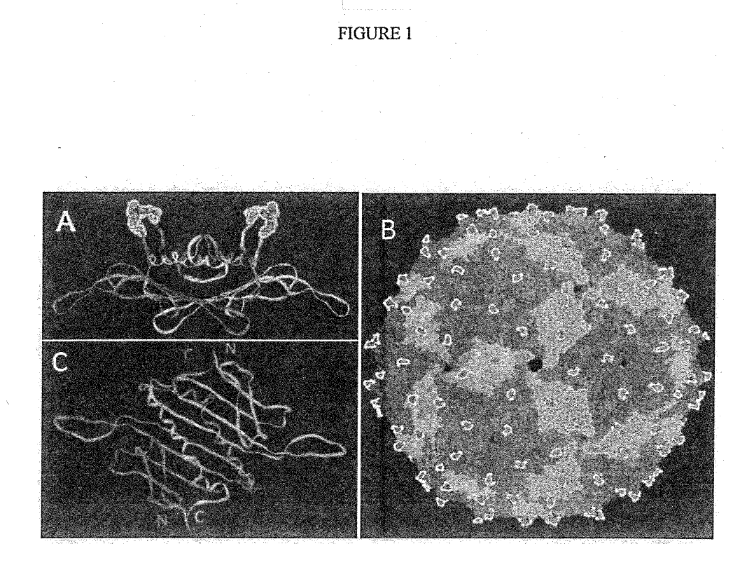

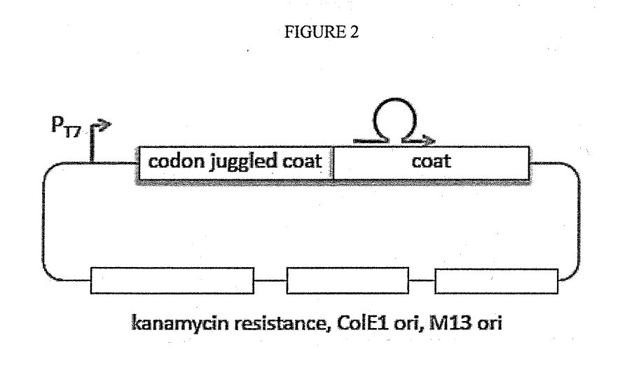

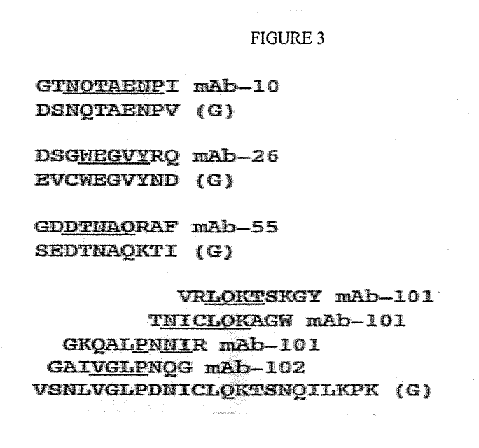

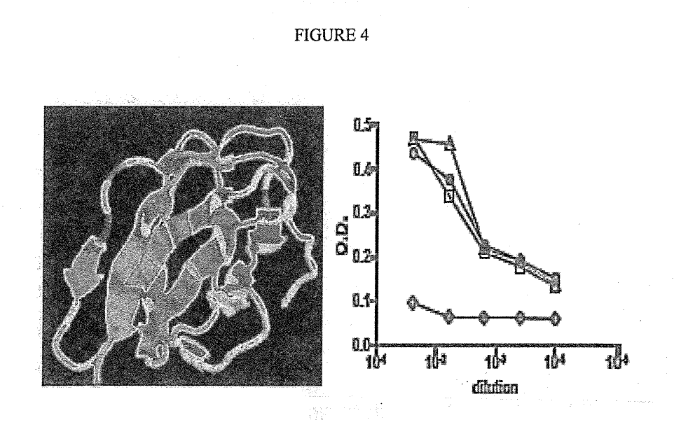

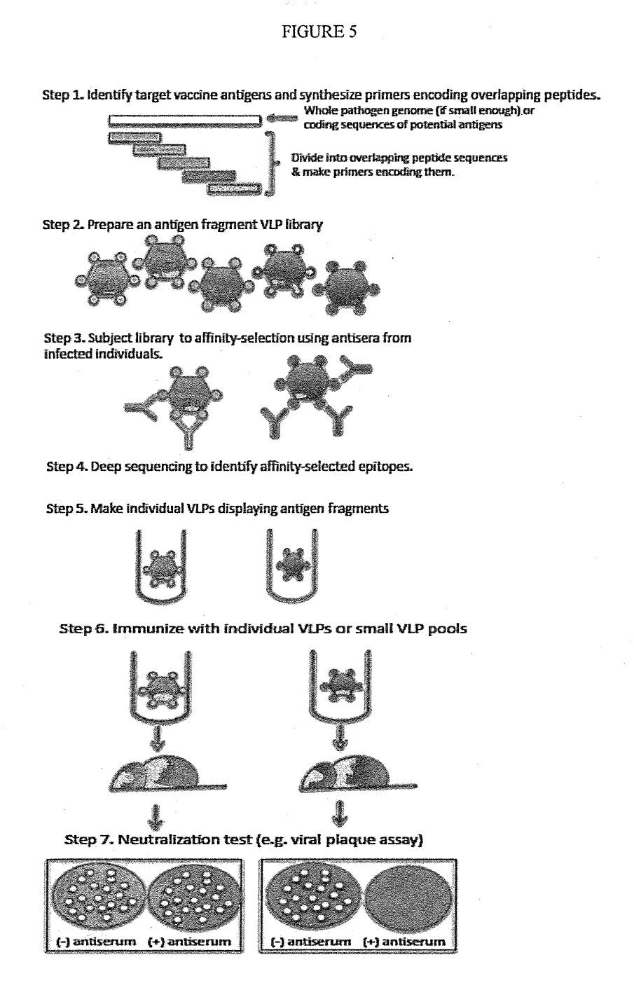

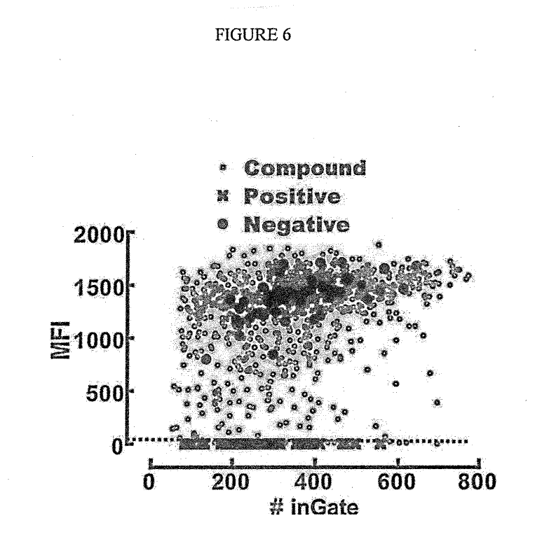

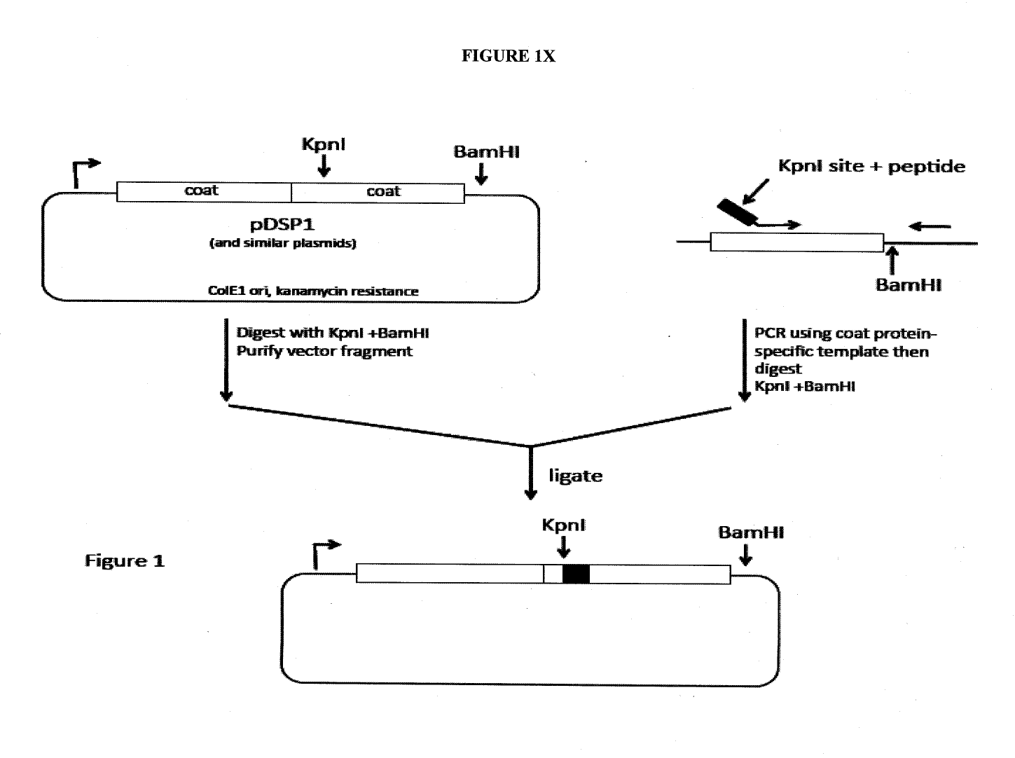

FIG. 1X depicts the pDSP1 plasmid and one technique for inserting a nucleic acid sequence encoding a heterologous peptide into the coat protein coding sequence. Note that pDSP1 contains the MS2 coat protein single-chain dimer sequence under control of the T7 promoter and transcription terminator. The plasmid replicates using a ColE1 origin of replication and confers resistance to kanamycin. The example shows utilization of a KpnI site in the AB-loop, but a similar scheme makes use of a SalI site just upstream of the AB-loop.

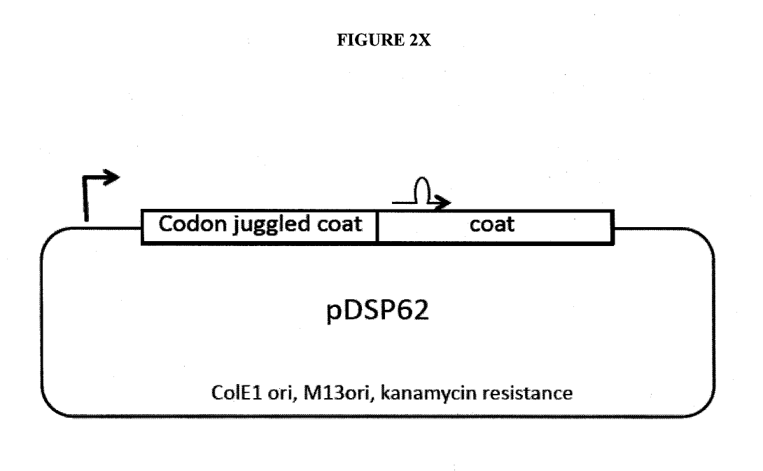

FIG. 2X depicts the pDSP62 plasmid, which is highly similar to pDSP 1 of FIG. 1. It differs from it in having a “codon-juggled” sequence in the 5′-half of the single-chain dimer. Another difference is the presence of an M13 origin of replication, which allows the facile preparation of single-strand plasmid DNA from cells superinfected with M13 phage. This enables an alternative to the method shown in FIG. 1 for insertion of foreign peptide sequences. A synthetic oligonucleotide containing a foreign sequence flanked by regions complementary to the insertion site is annealed to the single-strand template DNA and the primer is extended using DNA polymerase. Note that the plasmid called pDSP7 differs from pDSP62 only in containing PP7 coat sequences in place of MS2 and is therefore not illustrated separately. Another plasmid called pDSP7K differs from pDSP7 in having the sequence for a KpnI site in the AB-loop of the downstream half of its single-chain dimer.

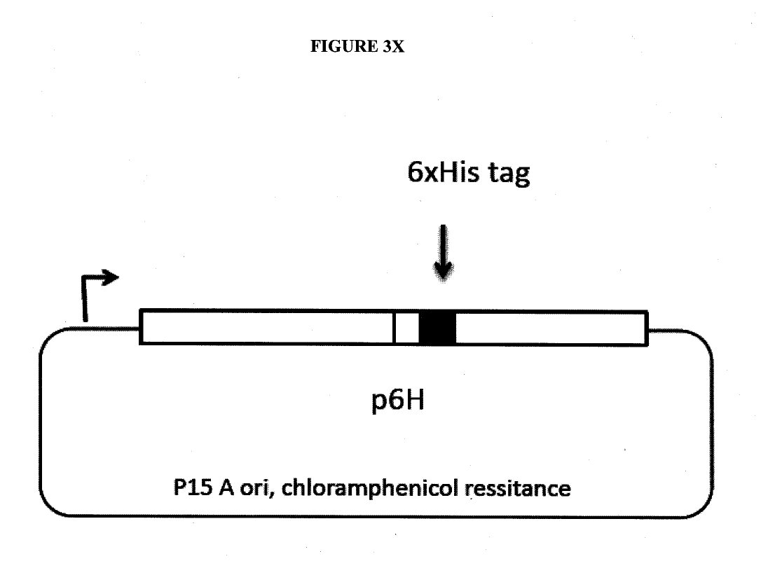

FIG. 3X shows a plasmid that produces a His6-tagged version of MS2 coat protein. In the example shown, the 6×His tag is inserted in the AB-loop of a single-chain coat protein dimer, but other configurations are possible, including fusion to N- or C-termini of either single-chain or conventional dimer coat proteins. The plasmid replicates from a P15A origin and is therefore compatible with pDSP1, pDSP62, pDSP7, pDSP7K, etc. which replicate from ColE1 origins. It confers resistance to chloramphenicol and expresses the 6×-His tagged single chain dimer coat protein from the T7 promoter and terminator. When present in cells together with other VLP producing plasmids (e.g. pDSP1) the two forms of coat protein co-assemble to produce a particle that can be purified on nickel affinity columns. A 6×His-tagged PP7 coat protein is produced from a similar plasmid for use with PP7 VLPs.

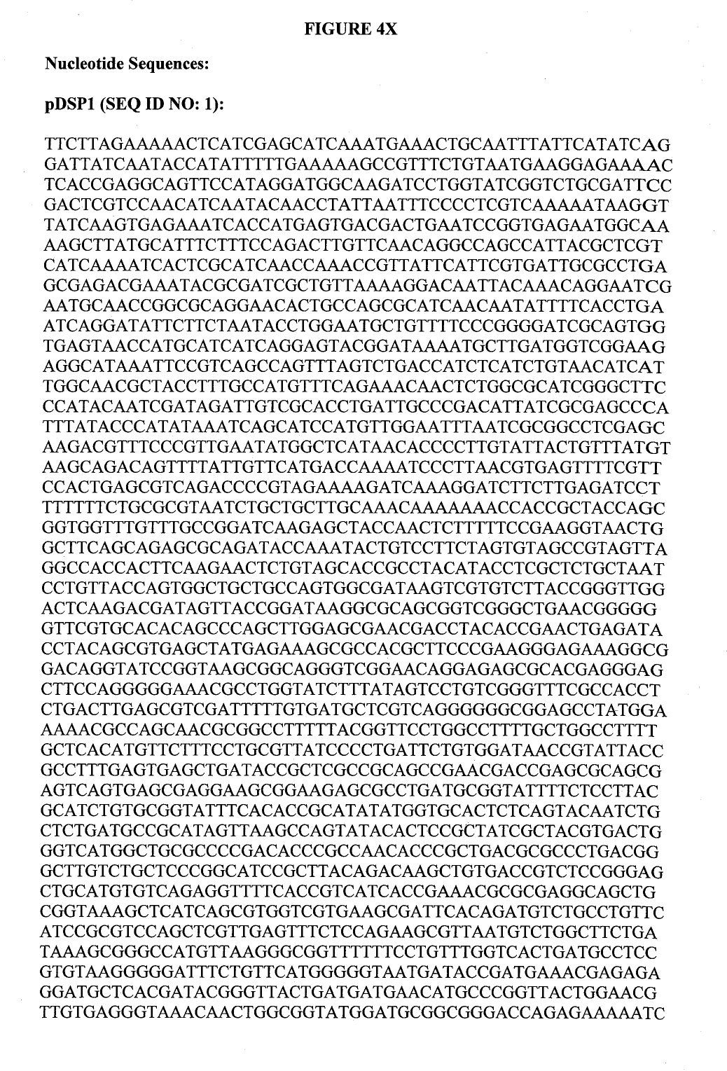

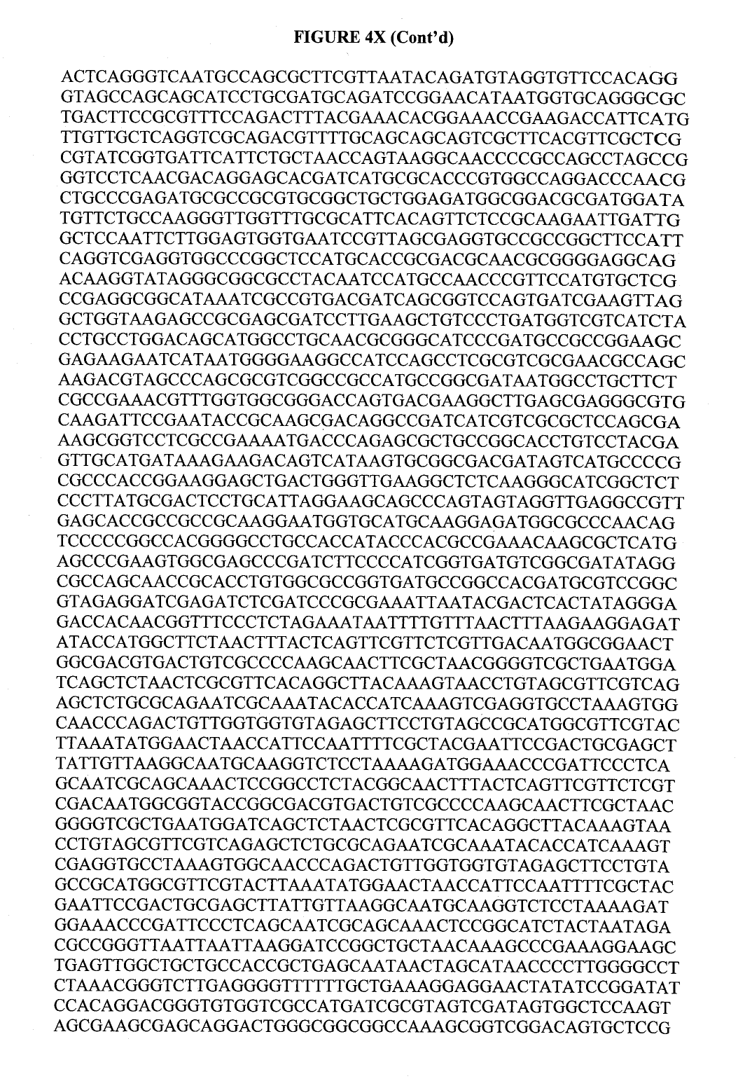

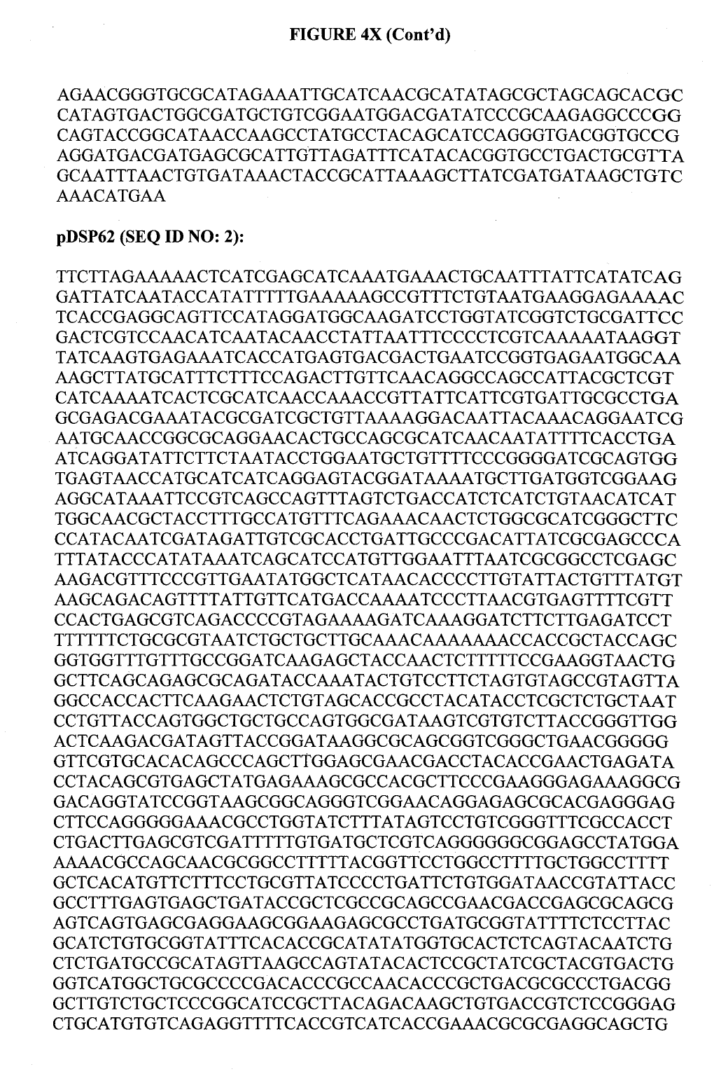

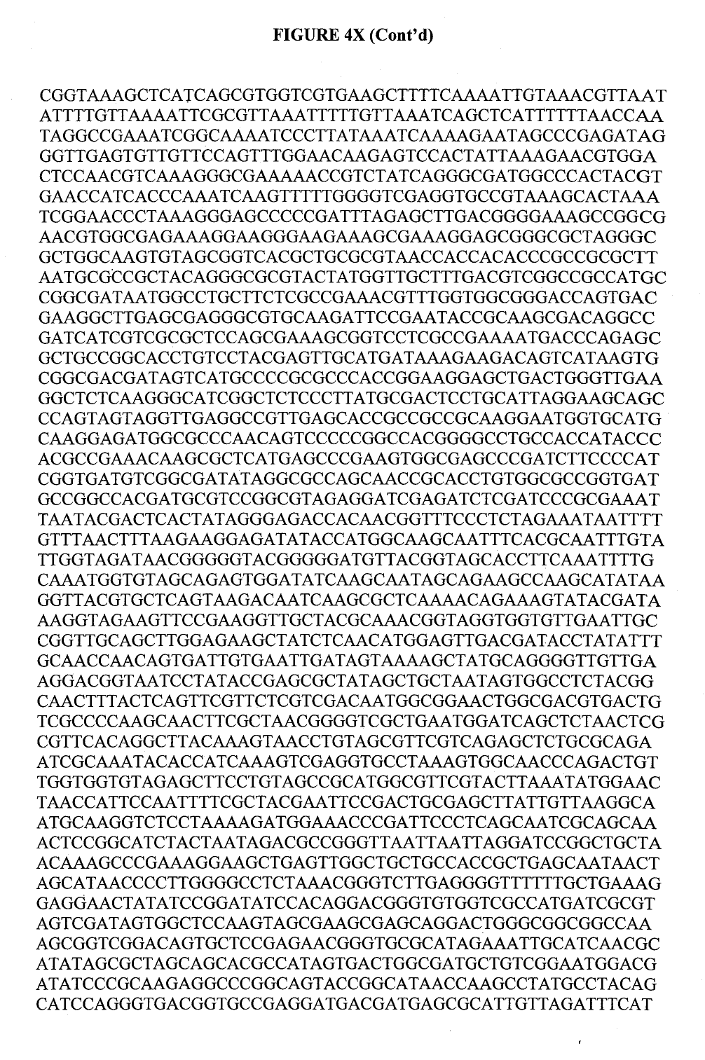

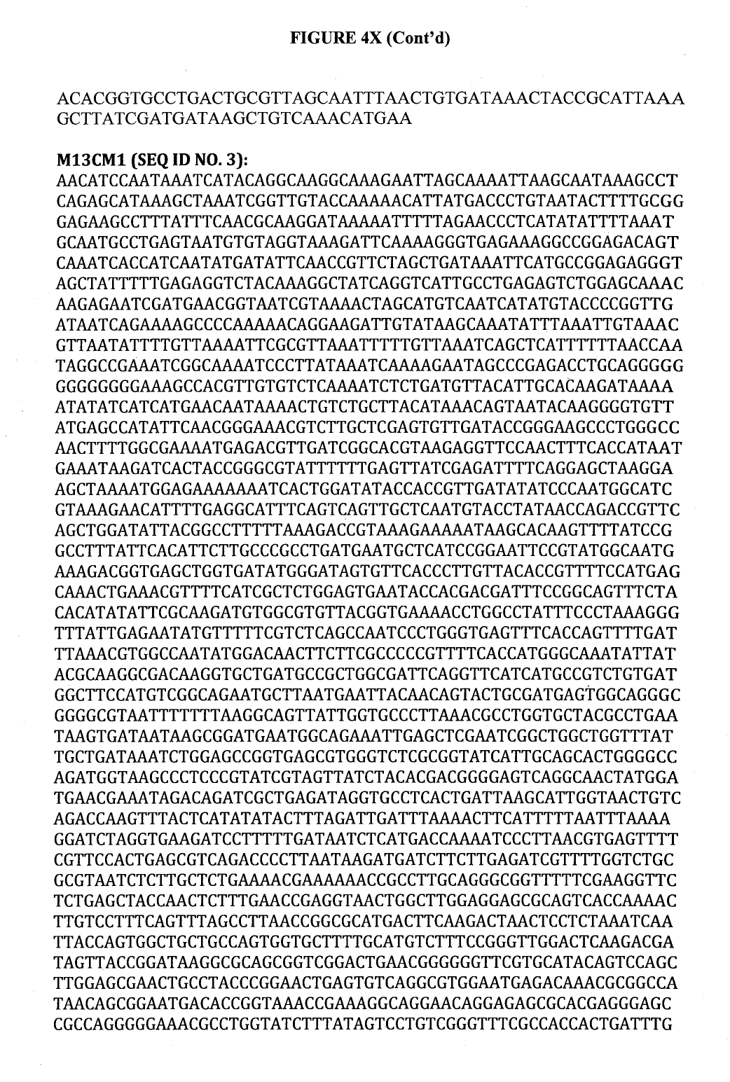

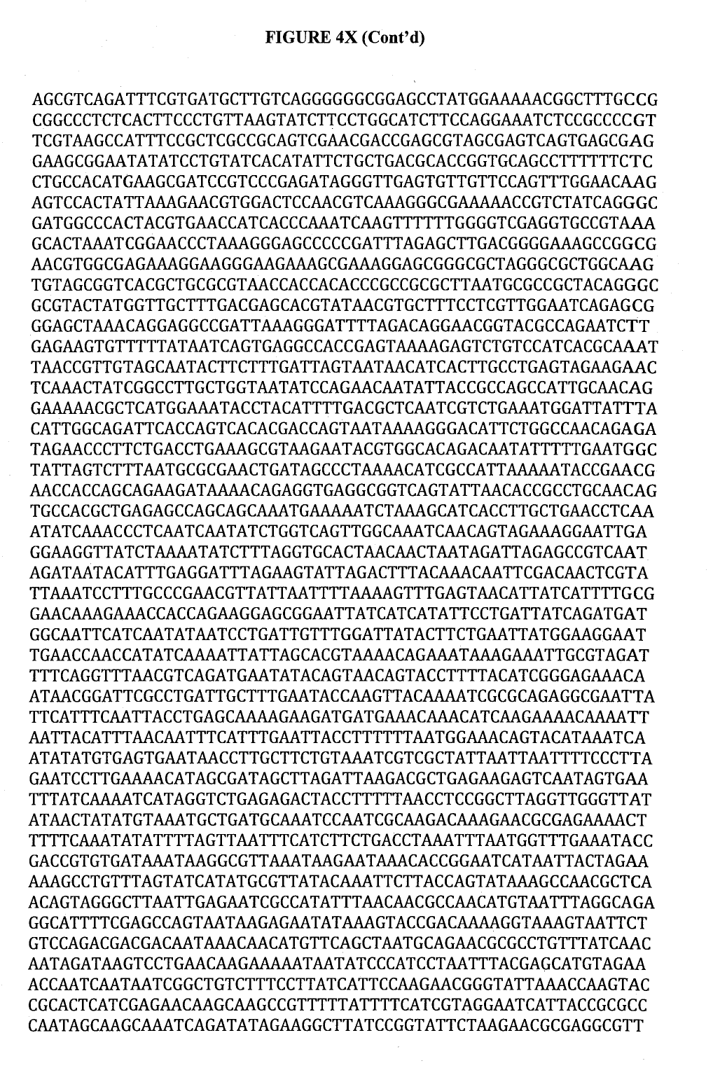

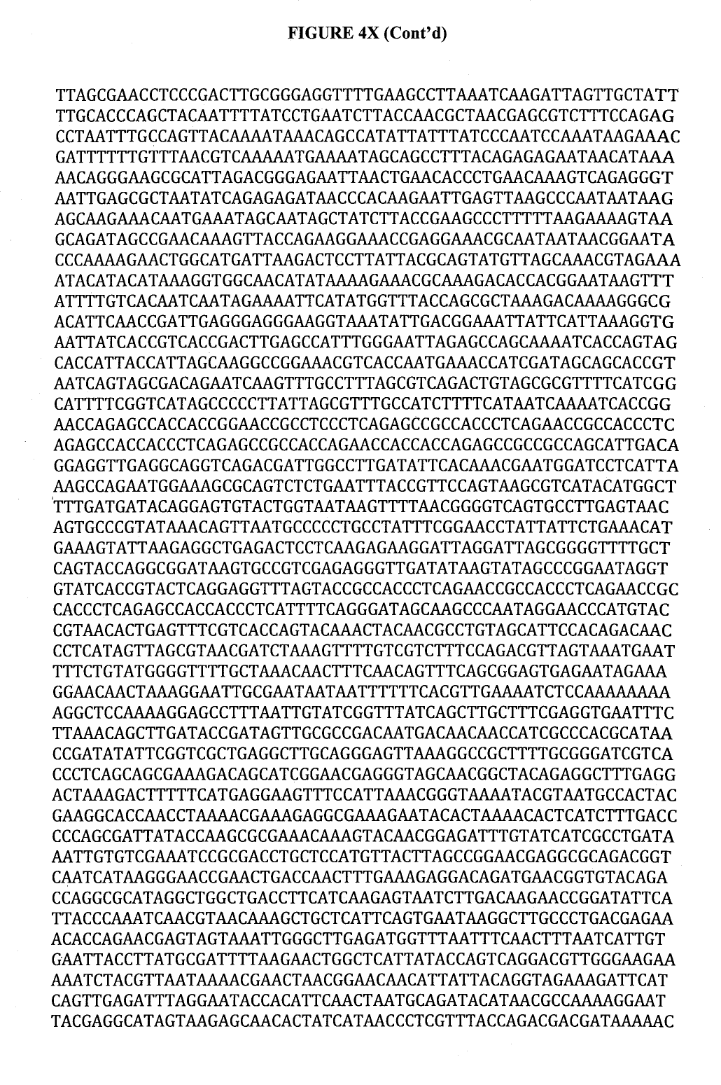

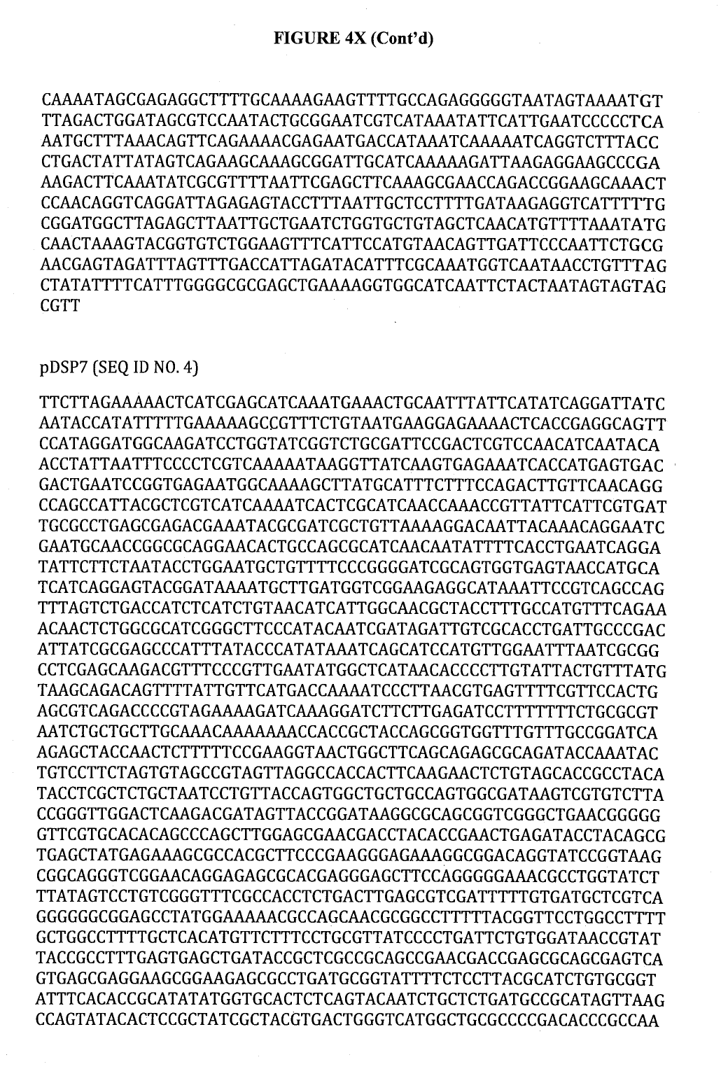

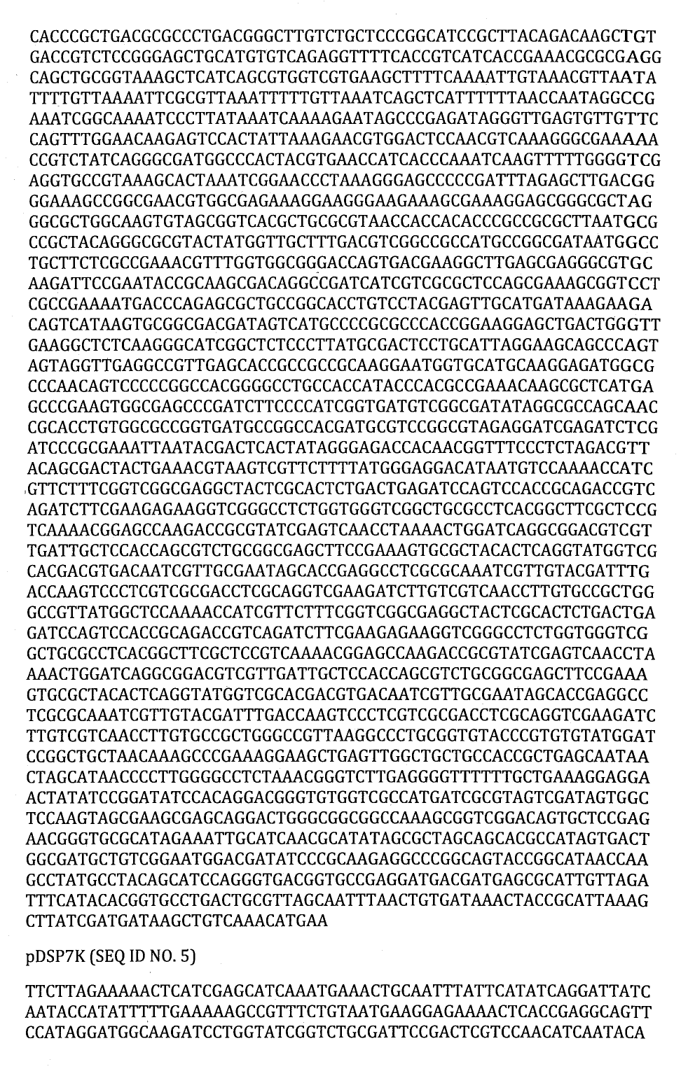

FIG. 4X contains the nucleic acid sequence for the pDSP1 plasmid (SEQ ID NO: 1), the nucleic acid sequence for the pDSP62 plasmid (SEQ ID NO: 2), and the nucleic acid sequence of M13CM1 (SEQ ID NO: 3.), a chloramphenicol resistant derivative of M13KO7 used as helper virus for production of single-strand DNA of pDSP1, pDSP62, pDSP7 or pDSP7K. Also provided are the sequences of pDSP7 (SEQ ID NO: 4), DSP7K (SEQ ID NO: 5) as well as p6H (SEQ ID NO: 6).

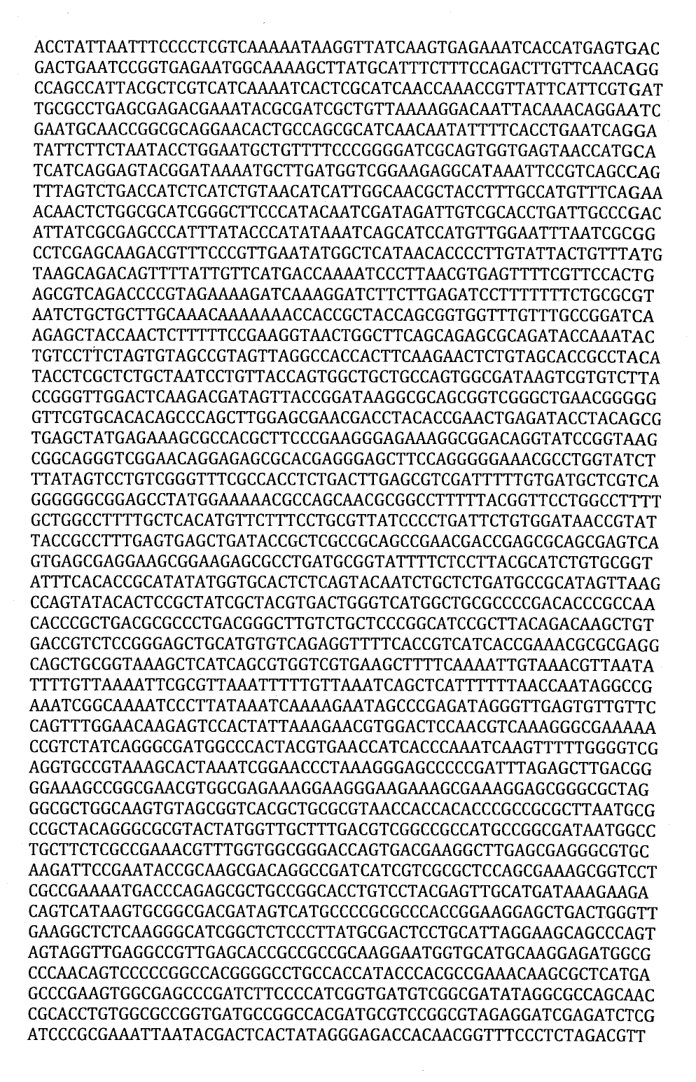

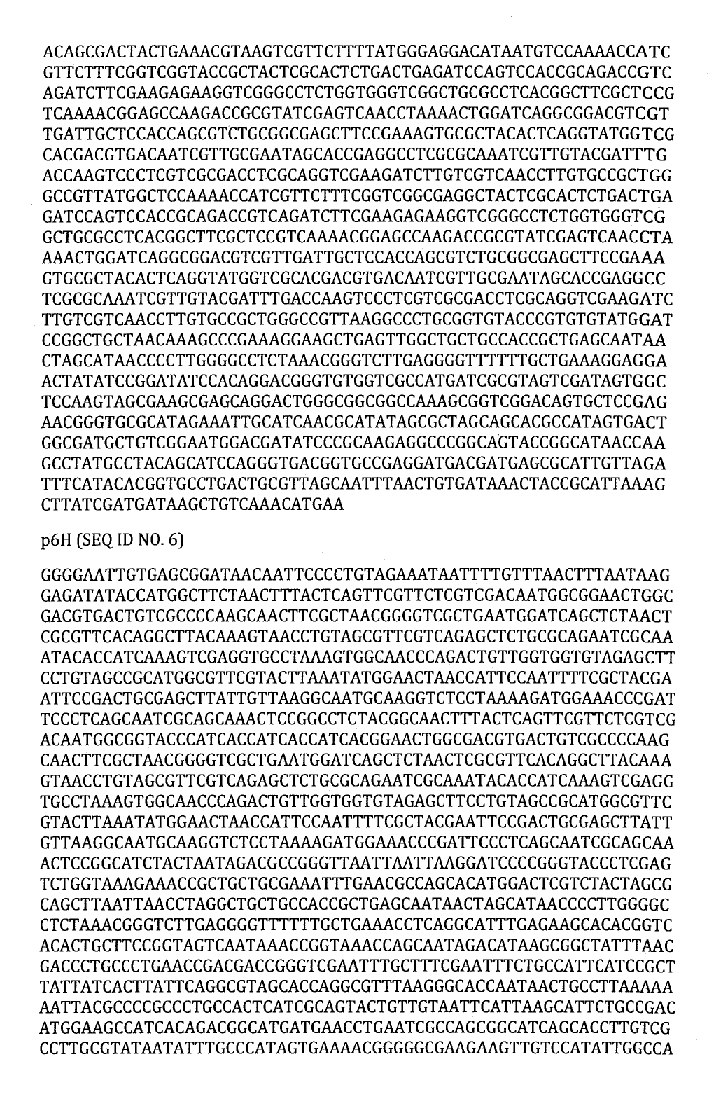

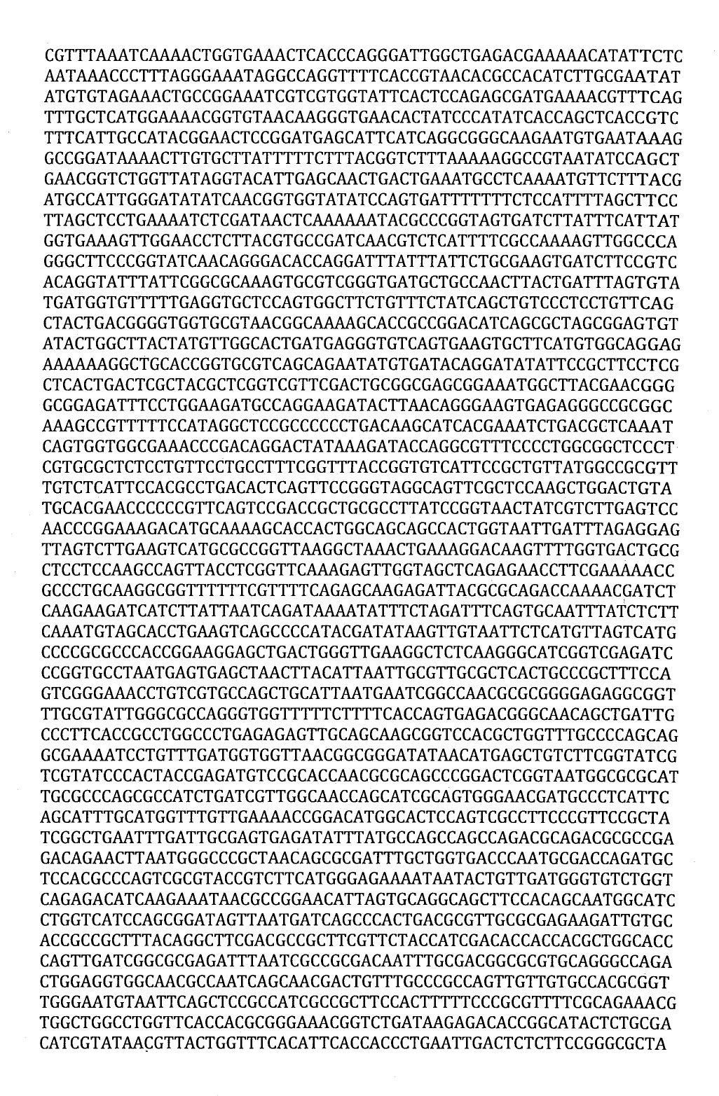

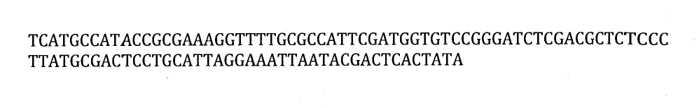

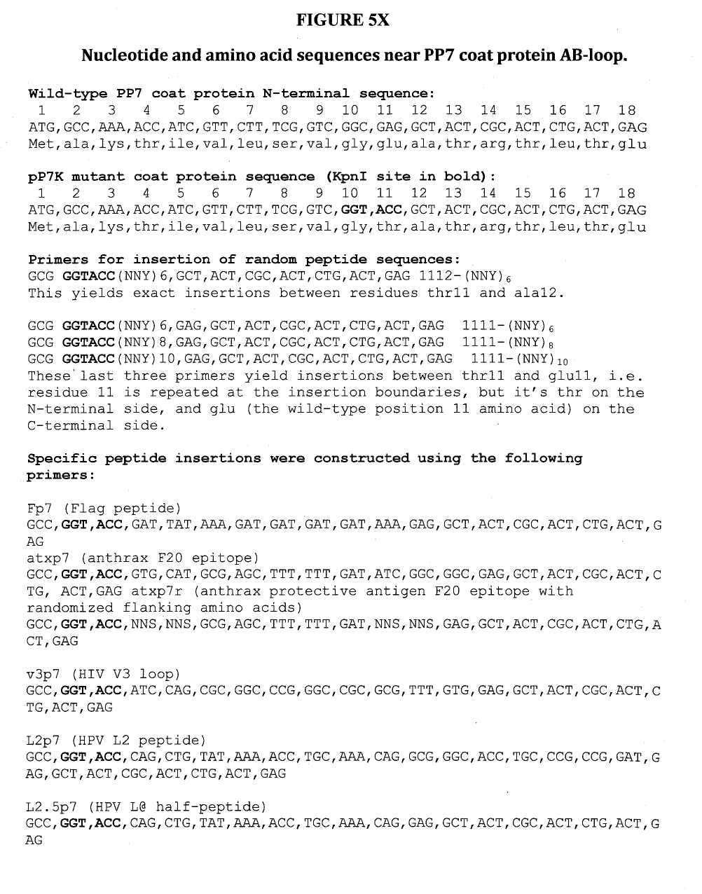

FIG. 5X provides the nucleotide and amino acid sequences near the PP7 coat protein AB-loop and also provides primer sequences that are used for peptide sequence insertion into plasmids like pDSP7K using the method illustrated for pDSP1 in FIG. 1.

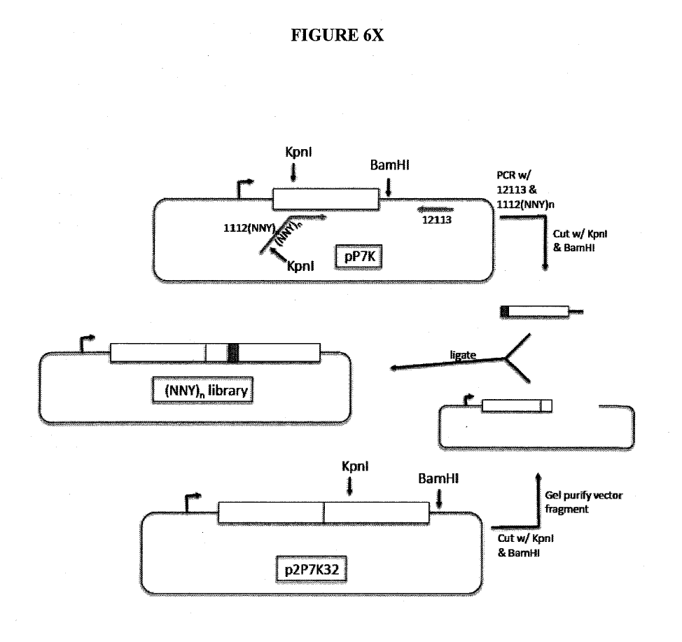

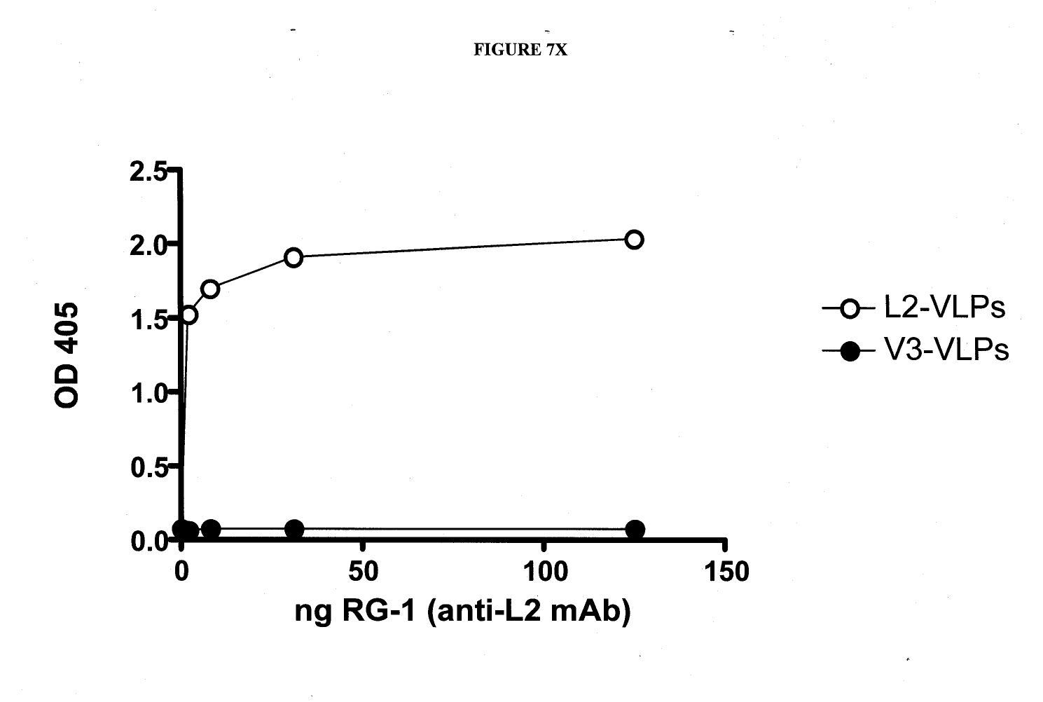

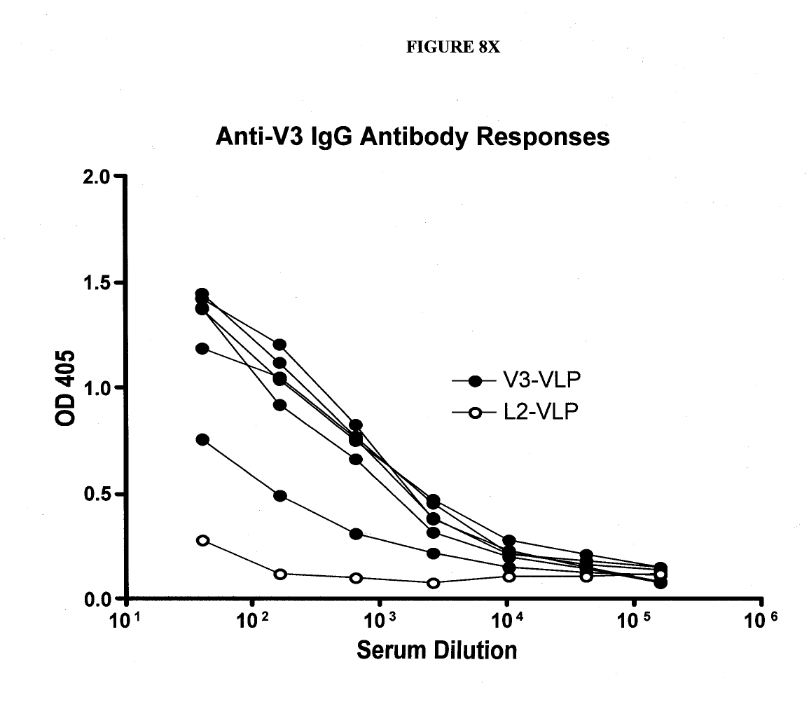

FIG. 6X illustrates a scheme that can be used for construction of antigen fragment libraries in plasmids like pDSP1, pDSP62, and pDSP7K, among others.

FIG. 7X illustrates that an anti-L2 mAb (RG-1) binds to PP7 L2-VLPs, but not PP7 V3-VLPs. Dilutions of mAb RG-1 were reacted with 500 ng/well of L2-VLPs or V3-VLPs. Binding was detected using a horseradish peroxidase-labeled goat anti-mouse IgG secondary followed by development with ABTS. Reactivity was determined by measurement of the absorbance at 405 nm (OD 405).

FIG. 8X illustrates that V3 peptide displaying PP7 VLPs induce anti-V3 IgG responses upon immunization. Shown are anti-V3 IgG antibody responses in mice immunized with PP7 V3-VLPs or, as a control, L2-VLPs. Mice were immunized three times with 10 μg of VLPs with incomplete Freund's adjuvant and then sera were collected two weeks after the final boost. Diluted sera from seven individual mice (six immunized with V3-VLPs and one immunized with L2-VLPs) were tested for reactivity with a peptide representing a portion of the V3 loop from HIV_{LAI} by ELISA. Binding was detected using a horseradish peroxidase-labeled goat anti-mouse IgG secondary followed by development with ABTS. Reactivity was determined by measurement of the absorbance at 405 nm (OD 405)

### DETAILED DESCRIPTION OF THE INVENTION

The Basis of Peptide Display and Affinity Selection on the RNA Phage VLP Platform.

It should be emphasized that the technology described here is based on virus-like particles that form when coat protein is expressed from a plasmid in bacteria. It is not based on the use of the infectious bacteriophage itself. By way of background, the normal RNA phage virion is comprised of an icosahedral capsid made of 180 copies of coat protein and one molecule of maturase protein together with one molecule of the RNA genome. Coat protein is also a specific RNA binding protein. Assembly may possibly be initiated when coat protein associates with its specific recognition target an RNA hairpin near the 5′-end of the replicase cistron. The virus particle is then liberated into the medium when the cell bursts under the influence of the viral lysis protein. The formation of an infectious virus requires at least three components, namely coat protein, maturase and viral genome RNA, but experiments show that the information required for assembly of the icosahedral capsid shell is contained entirely within coat protein itself. For example, purified coat protein can form capsids in vitro in a process stimulated by the presence of RNA [Beckett et al., 1988, J. Mol. Biol 204: 939-47]. Moreover, coat protein expressed in cells from a plasmid assembles into a virus-like particle in vivo [Peabody, D. S., 1990, J Biol Chem 265: 5684-5689]. The capability of coat protein to self-assemble into a VLP in the absence of other viral components forms the basis of this VLP-display technology. Our method relies on the production of VLPs when MS2 or PP7 coat protein is expressed from the plasmids described elsewhere in this application, or from other plasmids containing some or all of the various elements described in the embodiments.

Although the coat proteins of any number of known RNA phages could be utilized for display purposes, the RNA phage VLP system described here has been applied using coat proteins from two of them, namely MS2 and PP7. The two proteins show only about 13% amino acid identity, but have similar tertiary structures and each has been engineered by these inventors for the display of foreign peptides on the surface of the corresponding VLP. The peptide display and affinity selection capabilities rely on (1) the identification of a site or sites in coat protein that tolerate peptide insertion or fusion, and (2) on the encapsidation of the RNA that encodes the coat protein (and guest peptide) from which the VLP is assembled. The inventors have previously described the construction of a single-chain dimer version of coat protein which is highly tolerant of foreign peptide insertions at a site on the surface of the VLP. They also reported that the VLP encapsidates the RNA that encodes it, thus making possible the recovery and amplification of affinity selected VLPs by reverse transcription and polymerase chain reaction. Plasmid vectors that facilitate the production of complex random sequence and antigen fragment libraries and their use for the affinity selection of peptide mimics of epitopes have also been described.

Detailed Background on the VLP Display System.

Plasmid vectors we call pDSP62 and pDSP7 (FIGS. 2 and 2A) facilitate the insertion of foreign sequences and enable the construction of high complexity random sequence peptide libraries on MS2 and PP7 VLPs, respectively. Beside the usual plasmid features of a drug resistance determinant (kanamycin) and a plasmid replication origin (from ColEI) the plasmids contain: (i) An M13 origin of replication to facilitate the production of circular single-stranded DNA templates for use in the high efficiency site-directed mutagenesis method of Kunkel. An insertion primer is extended on single-stranded dUTP-substituted template DNA followed by ligation to produce closed circular DNA. Transformation of an ung⁺ E. coli strain results in strong selection for insertions. (ii) A single-chain dimer coat sequence with a synthetic “codon-juggled” upstream half. High tolerance of coat protein folding to the presence of AB-loop insertions depends on insertion into only one AB-loop of a single-chain dimer. In order to direct the annealing of a mutagenic primer to the targeted AB-loop we replaced the upstream half with a synthetic “codon-juggled” version of coat protein containing the maximum possible number of silent nucleotide substitutions. (iii) Valency Control. At high peptide density it is difficult for affinity selection to discriminate genuine high affinity peptides from those with weaker intrinsic affinities that bind tightly through multiple interactions (i.e. affinity vs. avidity). In our system valency control is achieved through the use of a variant of pDSP62 called pDSP62(am) (or pDSP7(am) in the case of PP7). It contains an amber codon at the junction between the two halves of the single-chain dimer. In the presence of a weak suppressor tRNA (expressed from pNMsupA) about 3% of the coat protein takes the form of the single-chain dimer with a foreign peptide in its downstream AB-loop. This co-assembles with an excess of the normally terminated wild-type protein to form mosaic capsids. Both forms of the protein are produced from a single mRNA, which is packaged by the VLP, thus preserving the genotype/phenotype linkage essential for affinity selection. After selection is complete, a return to high valency (and high immunogenicity) is achieved by simply cloning the selected sequences again in pDSP62, or, even more simply, by expressing the pDSP62(am) construct in the presence of an alternate suppressor tRNA with nearly 100% suppression efficiency.

Vaccine Discovery by VLP Display.

Our platform enables the production of vaccine candidates by two different routes.

1. Affinity Selection of Epitope Mimics (Mimotopes).

Introduced about 1985, phage display offered the possibility of a fairly direct route to development of peptide vaccines through the discovery of epitopes by affinity selection on antibody targets, and their presentation to the immune system as immunogens. In fact, phage display methods generally work well for selection of conformationally unconstrained linear epitopes, but, despite some hints of success, immunogenic mimics of more complex epitopes have been much more challenging. A quirk of filamentous phage biology makes it difficult to display peptides at the very high-densities necessary for best immunogenicity. This means that epitope mimics discovered by phage display are normally produced synthetically and then attached to a different, more immunogenic carrier. Peptide ligands often lose much of their activity for the selecting antibody when transferred to new environments, with an attendant loss of ability to elicit the desired antibody response. RNA phage VLPs integrate the epitope discovery/optimization and the immunization functions into a single particle, so that the structural constraints present during affinity selection are strictly maintained during immunization.

2. Rational Design by Display of Epitopes Already Defined.

This approach is illustrated by our creation of a second generation HPV vaccine. The reader will no doubt be aware of the recently introduced Human Papilloma Virus vaccines, which rely on immunization with the major HPV capsid protein, L1. Unfortunately, L1 is genetically variable, so the vaccines protect against only a few of the hundred or so HPV serotypes. The minor capsid protein, L2, although much more conserved, is not normally immunogenic. Nevertheless, when presented in an immunogenic form L2 elicits an HPV-neutralizing antibody response, thus raising the possibility of single component vaccine that could neutralize a much wider range of HPV serotypes. We introduced a 17-amino acid peptide from a well-conserved region of L2 into the AB-loop of the PP7 VLP. Animals immunized with the VLP mounted high titer anti-L2 responses, and were protected against infection by HPV pseudovirions with highly divergent serotypes.

Epitope Profiling and Discovery of Vaccines and Diagnostic Reagents Using the VLP Platform.

The present invention utilizes the ability to conduct both affinity selection and immunization on the RNA phage VLP to create an “epitope profile” of the antibody response to a given antigen based on affinity selection of peptide recognized by antisera, thus identifying epitopes that may be directly useful as vaccines or as diagnostic reagents.

In addition to pathogens, the following represent additional, nonlimiting examples of approaches to creating various epitopic profiles which may be useful in therapeutic and/or diagnostic aspects of the present invention.

1. Fine mapping (human serum or tracheal aspirate) IgE responses to important allergens from the environment. These could ultimately encompass the large array of known allergens of cats, housemites, peanuts, etc. It is quite likely that even when environmental allergens can't be absolutely identified, the VLPs and methods according to the present invention could put forth a candidate allergen by BLASTing peptides onto the NCBI nonredundant library. VLPs generated in such a way could potentially serve as vaccine immunogens to raise an IgG response to allergens that would reduce allergic responses.

2. Responses to immunization. Presently there is no robust means to compare and contrast responses to natural infection (e.g. measles, mumps, rubella or pertussus) with vaccine-induced antibody responses. There may be a number of fine differences that the MS2/PP7 VLP display technology according to the present invention could (with reasonable facility) uncover. Distinguishing responses to vaccines vs natural infection is of keen interest, inasmuch as providing a vaccine response which mimics a natural immune response may be particularly desirable in certain instances; and

3. Responses to foreign antigens and alloimmunization. It has been known for many years that some hemophiliacs respond to factor infusions by raising a neutralizing antibody response, since their bodies have never seen Factor VIII or IX in the past. This is one of many responses that patients will generate when exposed to foreign, or allogeneic antigens. The epitopes involved in responses to coagulation factors and infusion of other corrective proteins in patients who do not express the proteins naturally, could be informative in vaccine and intervention approaches. Allogeneic responses is a very important category. When patients are exposed to alloantigens consequent to transfusion or transplantation, they frequently raise immune responses including B cell responses, against the allogeneic antigens. The VLP platform and methods according to the present invention could be used to dissect, as never before, the exact molecular recognition events that lead to allogeneic responses to specific allogeneic antigens. These antigens could include both CHO and protein based epitopes emerging from ABO, Rh, Duffy, Kell, Kidd, Lutheran, P, Lewis, MNS, HLA, KIR, and many other polymorphic alloantigen groups, and the VLPs and methods according to the present invention could be used to determine the exact nature of the immune response, therefore suggesting ways to intervene against that response.

Thus, while a principal focus is in the infectious disease area, the present invention may be used in numerous applications outside of the infectious disease area, in order to facilitate the development of high-impact diagnoses and therapeutics outside of the infectious disease applications.

Below are described the specific steps in the epitope profiling process. Dengue Virus is used here as a convenient example, but the reader should remember that the same procedure can be applied to any pathogen, allergen, or antigen. Note also that the specific peptide lengths, overlaps, library construction techniques, etc. are also subject to adjustment over a wide range, and the values and parameters presented here are only examples.

Antigen Fragment Library Construction.

Knowledge of the Dengue Virus genome sequence and the existence of massively parallel microchip-based synthesis methods capable of making hundreds of thousands of specific oligonucleotides allows the efficient construction of antigen fragment libraries. The oligonucleotides are designed by virtually dividing the viral proteome into a large series of overlapping 10-amino acid fragments. After silent mutation of any codons rarely found in E. coli, the peptide-encoding sequences are then flanked with 18-nucleotide sequences that anneal the primers to the site of insertion in coat protein's AB-loop (see FIGS. 1 & 2). The relatively small size of the Dengue proteome allows the production of oligonucleotides that scan the entire genome with a series of peptides, progressing through the sequence in a series of small steps. For example, scanning the entire genome with 10-mers at 1-amino acid increments (i.e. with 9-amino acid overlaps) requires only about 4,000 primers, a number well within the capabilities of the new mega-scale synthesis methods. A library with, say, a ten-fold over-representation of the genome requires only around 40,000 clones, a number many orders of magnitude below the current capabilities of our VLP display system. The use of synthetic DNA to site-directedly mutagenize pDSP62—as opposed, for example, to the cloning of randomly generated DNase I fragments of the viral genome—lets us precisely control reading frame, peptide insertion length, overlap, and codon usage. pDSP62 was designed specifically to utilize oligonucleotide primers for efficient synthesis of high complexity peptide libraries, and we have already synthesized random sequence libraries with more than 10¹⁰ individual clones. These complexities far exceed the requirements of the experiments proposed here.

Some pathogens, of course, have much larger genomes than Dengue's. Existing oligonucleotide synthesis methods allow for the production of around 55,000 independent sequences on a single chip, and this number is bound to increase as the technology advances. This means that libraries containing perhaps a half-million to a million individual antigen fragments are already possible using existing methods. Increases in the average size of displayed peptides, or decreases in the extent of their overlap reduce the number of individual sequences necessary to represent the entire peptidome of an organism, thus increasing the size of the peptidome the method can cover. Even so in many instances it will make sense to target only those proteins that are expressed on a pathogen's surface, or are otherwise implicated as having antigenic significance.

It should also be noted that other methods for construction of antigen-fragment libraries exist. They typically rely on actual random fragmentation of DNA representing the genome (or antigen) of interest, followed by insertion into the display site. These methods can be used, of course, but for most applications they must be considered inferior to the one described above.

Random Sequence Library Construction. We have previously elaborated methods for construction of complex libraries of random sequence peptides on MS2 VLPs. They basically involve the use of recombinant DNA and site-directed mutagenesis methods to introduce random oligonucleotides encoding random peptides into the coat protein gene. For example, the introduction of 10 copies of the triplet sequence NNS (where N is A, C, G, or T, and where S is G or C) results in 10 mer peptide sequences where the 20 amino acids occur randomly. Libraries with tens to hundreds of billions of individual clones, each of which carries a different randomly generated sequence, can be constructed using existing methods.

Antigen fragment libraries have the advantage of relative sequence simplicity, since, in our example, they contain only sequences found in the Dengue antigens themselves. Antigen fragment libraries based on short peptides suffer the disadvantage however, that they normally allow the presentation only of linear epitopes. Random sequence libraries contain much more sequence complexity and introduce the possibility that affinity selection can find peptide mimics of protein conformational and carbohydrate epitopes, thus expanding the capabilities of the system.

Affinity-Selection.

A number of methods for affinity-selection have been described in the literature. The basic idea is to immobilize the selecting antibodies on the surface of a plastic dish or on small beads (e.g. protein G beads) and to them incubate them with the appropriate VLP library. VLPs displaying peptides recognized by one or more antibodies will be retained when unbound VLPs are washed away. The bound sequences are the eluted and the RNAs they contain are amplified by reverse transcription and polymerase chain reaction. The resulting DNA may be recloned for another round of VLP display and selection or subjected directly to sequence analysis.

Characterization of Affinity-Selected Sequences by Deep Sequence Analysis.

New generation DNA sequencing technologies (e.g. Ion Torrent) allow the simultaneous determination of the sequences of millions of individuals in a VLP selectant population. This allows the complete characterization of the peptide epitope mimics recognized by a given serum sample, even those that occur only rarely, and enables the construction of an epitope profile of the serum in question. Each of these epitope-displaying VLPs represents a potential vaccine candidate or diagnostic reagent. Taking Dengue Virus as a specific example, individual epitope-VLPs can be tested for the ability to elicit a Dengue neutralizing response by immunization of animals or humans. Such epitopes may be common to diverse Dengue Virus strains, or specific for different serotypes, making them useful as reagents for determining whether a person is infected Dengue, and if so, with which serotype.

Immunization and Neutralization Tests of VLP Vaccine Candidates.

Once identified as described above, individual peptide-displaying VLPs are purified and used as immunogens in an appropriate animal or human model. The antibodies they elicit are then tested for the ability to react with and neutralize the target antigen. In the case of the Dengue Virus, for example, antisera could be tested for the ability to inhibit infection of cells in culture using a so-called Plaque Reduction Neutralization Test (or PRNT). VLPs able to elicit a neutralizing response would be further tested, ultimately in clinical trials, for their efficacy as a vaccine.

Use of VLPs as Diagnostic Reagents.

Since the process described above identifies a number of VLPs displaying peptides reactive for antibodies elicited during antigen exposure (e.g. during Dengue Virus infection) these VLPs represent potential diagnostic reagents for detection of exposure to the Dengue Virus. Moreover, because different dengue serotypes present a somewhat different spectrum of epitopes, each may elicit a different spectrum of antibody specificities. Since the collection of VLPs identified by the above procedure should contain some that are serotype specific, they may be useful as reagents to determine whether an individual has been infected with Dengue, and if so, with which serotype. In one embodiment, the VLPs are arrayed on a chip such that those commonly recognized by IgM responses against dengue 1 are in one region of the chip, those recognized by anti-dengue 2 IgM in another region, and so on, with binding by a given antiserum detected with an anti-human IgM antibody conjugate. As a result, rapid single-test serotype-specific diagnosis is possible. Again, Dengue Virus serves only as an example. These general methods should apply to antigens of any type.

Some Definitions and General Considerations:

In accordance with the present invention there may be employed conventional molecular biology, microbiology, and recombinant DNA techniques within the skill of the art. Such techniques are explained fully in the literature. See, e.g., Sambrook et al, 2001, “Molecular Cloning: A Laboratory Manual”; Ausubel, ed., 1994, “Current Protocols in Molecular Biology” Volumes I-III; Celis, ed., 1994, “Cell Biology: A Laboratory Handbook” Volumes I-III; Coligan, ed., 1994, “Current Protocols in Immunology” Volumes I-III; Gait ed., 1984, “Oligonucleotide Synthesis”; Hames & Higgins eds., 1985, “Nucleic Acid Hybridization”; Hames & Higgins, eds., 1984, “Transcription And Translation”; Freshney, ed., 1986, “Animal Cell Culture”; IRL Press, 1986, “Immobilized Cells And Enzymes”; Perbal, 1984, “A Practical Guide To Molecular Cloning.”

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise.

Furthermore, the following terms shall have the definitions set out below.

As used herein, the term “polynucleotide” refers to a polymeric form of nucleotides of any length, either ribonucleotides or deoxyribonucleotides, and includes both double- and single-stranded DNA and RNA. A polynucleotide may include nucleotide sequences having different functions, such as coding regions, and non-coding regions such as regulatory sequences (e.g., promoters or transcriptional terminators). A polynucleotide can be obtained directly from a natural source, or can be prepared with the aid of recombinant, enzymatic, or chemical techniques. A polynucleotide can be linear or circular in topology. A polynucleotide can be, for example, a portion of a vector, such as an expression or cloning vector, or a fragment.

As used herein, the term “polypeptide” refers broadly to a polymer of two or more amino acids joined together by peptide bonds. The term “polypeptide” also includes molecules which contain more than one polypeptide joined by a disulfide bond, or complexes of polypeptides that are joined together, covalently or noncovalently, as multimers (e.g., dimers, tetramers). Thus, the terms peptide, oligopeptide, and protein are all included within the definition of polypeptide and these terms are used interchangeably. It should be understood that these terms do not connote a specific length of a polymer of amino acids, nor are they intended to imply or distinguish whether the polypeptide is produced using recombinant techniques, chemical or enzymatic synthesis, or is naturally occurring.

The amino acid residues described herein are preferred to be in the “L” isomeric form. However, residues in the “D” isomeric form can be substituted for any L-amino acid residue, as long as the desired functional is retained by the polypeptide. NH₂ refers to the free amino group present at the amino terminus of a polypeptide. COOH refers to the free carboxy group present at the carboxy terminus of a polypeptide.

The term “coding sequence” is defined herein as a portion of a nucleic acid sequence which directly specifies the amino acid sequence of its protein product. The boundaries of the coding sequence are generally determined by a ribosome binding site (prokaryotes) or by the ATG start codon (eukaryotes) located just upstream of the open reading frame at the 5′-end of the mRNA and a transcription terminator sequence located just downstream of the open reading frame at the 3′-end of the mRNA. A coding sequence can include, but is not limited to, DNA, cDNA, and recombinant nucleic acid sequences.

A “heterologous” region of a recombinant cell is an identifiable segment of nucleic acid within a larger nucleic acid molecule that is not found in association with the larger molecule in nature.

An “origin of replication” refers to those DNA sequences that participate in DNA synthesis.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation, as well as protein binding domains (consensus sequences) responsible for the binding of RNA polymerase. Eukaryotic promoters will often, but not always, contain “TATA” boxes and “CAT” boxes. Prokaryotic promoters contain Shine-Dalgarno sequences in addition to the −10 and −35 consensus sequences.

An “expression control sequence” is a DNA sequence that controls and regulates the transcription and translation of another DNA sequence. A coding sequence is “under the control” of transcriptional and translational control sequences in a cell when RNA polymerase transcribes the coding sequence into mRNA, which is then translated into the protein encoded by the coding sequence. Transcriptional and translational control sequences are DNA regulatory sequences, such as promoters, enhancers, polyadenylation signals, terminators, and the like, that provide for the expression of a coding sequence in a host cell.

A “signal sequence” can be included before the coding sequence. This sequence encodes a signal peptide, N-terminal to the polypeptide, that communicates to the host cell to direct the polypeptide to the cell surface or secrete the polypeptide into the media, and this signal peptide is clipped off by the host cell before the protein leaves the cell. Signal sequences can be found associated with a variety of proteins native to prokaryotes and eukaryotes.

A cell has been “transformed” by exogenous or heterologous DNA when such DNA has been introduced inside the cell. The transforming DNA may or may not be integrated (covalently linked) into chromosomal DNA making up the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA.

It should be appreciated that also within the scope of the present invention are nucleic acid sequences encoding the polypeptide(s) of the present invention, which code for a polypeptide having the same amino acid sequence as the sequences disclosed herein, but which are degenerate to the nucleic acids disclosed herein. By “degenerate to” is meant that a different three-letter codon is used to specify a particular amino acid.

As used herein, “epitope” refers to an antigenic determinant of a polypeptide. An epitope could comprise 3 amino acids in a spatial conformation which is unique to the epitope. Generally an epitope consists of at least 5 such amino acids, and more usually, consists of at least 8-10 such amino acids. Methods of determining the spatial conformation of amino acids are known in the art, and include, for example, x-ray crystallography and 2-dimensional nuclear magnetic resonance.

As used herein, a “mimotope” is a peptide that mimics an authentic antigenic epitope.

As used herein, the term “coat protein(s)” refers to the protein(s) of a bacteriophage or a RNA-phage capable of being incorporated within the capsid assembly of the bacteriophage or the RNA-phage.

As used herein, a “coat polypeptide” as defined herein is a polypeptide fragment of the coat protein that possesses coat protein function and additionally encompasses the full length coat protein as well or single-chain variants thereof.

As used herein, the term “immune response” refers to a humoral immune response and/or cellular immune response leading to the activation or proliferation of B- and/or T-lymphocytes and/or and antigen presenting cells. In some instances, however, the immune responses may be of low intensity and become detectable only when using at least one substance in accordance with the invention. “Immunogenic” refers to an agent used to stimulate the immune system of a living organism, so that one or more functions of the immune system are increased and directed towards the immunogenic agent. An “immunogenic polypeptide” is a polypeptide that elicits a cellular and/or humoral immune response, whether alone or linked to a carrier in the presence or absence of an adjuvant. Preferably, antigen presenting cell may be activated.

As used herein, the term “self antigen” refers to proteins encoded by the host's DNA and products generated by proteins or RNA encoded by the host's DNA are defined as self. In addition, proteins that result from a combination of two or several self-molecules or that represent a fraction of a self-molecule and proteins that have a high homology two self-molecules as defined above (>95%, preferably >97%, more preferably >99%) may also be considered self. Examples of a self-antigen includes but is not limited to ErbB-2, amyloid-beta, immunoglobulin E (IgE), gastrin, ghrelin, vascular endothelial growth factor (VEGF), interleukin (IL)-17, IL-23, IL-13, CCR5, CXCR4, nerve growth factor (NGF), angiotensin II, TRANCE/RANKL and MUC-1.

As used herein, the term “vaccine” refers to a formulation which contains the composition of the present invention and which is in a form that is capable of being administered to an animal.

As used herein, the term “virus-like particle of a bacteriophage” refers to a virus-like particle (VLP) resembling the structure of a bacteriophage, being non replicative and noninfectious, and lacking at least the gene or genes encoding for the replication machinery of the bacteriophage, and typically also lacking the gene or genes encoding the protein or proteins responsible for viral attachment to or entry into the host.

This definition should, however, also encompass virus-like particles of bacteriophages, in which the aforementioned gene or genes are still present but inactive, and, therefore, also leading to non-replicative and noninfectious virus-like particles of a bacteriophage.

VLP of RNA bacteriophage coat protein: The capsid structure formed from the self-assembly of one or more subunits of RNA bacteriophage coat protein and optionally containing host RNA is referred to as a “VLP of RNA bacteriophage coat protein”. In a particular embodiment, the capsid structure is formed from the self assembly of 1-180 subunits.

A nucleic acid molecule is “operatively linked” to, or “operably associated with”, an expression control sequence when the expression control sequence controls and regulates the transcription and translation of nucleic acid sequence. The term “operatively linked” includes having an appropriate start signal (e.g., ATG) in front of the nucleic acid sequence to be expressed and maintaining the correct reading frame to permit expression of the nucleic acid sequence under the control of the expression control sequence and production of the desired product encoded by the nucleic acid sequence. If a gene that one desires to insert into a recombinant DNA molecule does not contain an appropriate start signal, such a start signal can be inserted in front of the gene.

The term “stringent hybridization conditions” are known to those skilled in the art and can be found in Current Protocols in Molecular Biology, John Wiley & Sons, N.Y. (1989), 6.3.1-6.3.6. A preferred, non-limiting example of stringent hybridization conditions is hybridization in 6× sodium chloride/sodium citrate (SSC) at about 45° C., followed by one or more washes in 0.2.×SSC, 0.1% SDS at 50° C., preferably at 55° C., and more preferably at 60° C. or 65° C.

### Production of Virus-Like Particles

The growth of recombinant DNA technology in recent years has led to the introduction of vaccines in which an immunogenic protein has been identified, cloned and expressed in a suitable host to obtain sufficient quantities of protein to allow effective protective immunization in both animals and humans. Many of the most effective vaccines are based on the potent ability of virion surfaces to elicit neutralizing antibodies. These include licensed killed or attenuated virus vaccines, such as polio, influenza and rabies, which effectively induce protective antibody responses. More recently, subunit vaccines based upon self-assemblages of the structural proteins of human papillomavirus (HPV) and hepatitis B virus (HBV) have been approved by the Food and Drug Administration.

Phage display is one of several technologies that make possible the presentation of large libraries of random amino acid sequences with the purpose of selecting from them peptides with certain specific functions. The basic idea is to create recombinant bacteriophage genomes containing a library of randomized sequences genetically fused to one of the structural proteins of the virion. When such recombinants are transfected into bacteria each produces virus particles that display a particular peptide on their surface and which package the same recombinant genome that encodes that peptide, thus establishing the linkage of genotype and phenotype essential to the method. Arbitrary functions (e.g. the binding of a receptor, immunogenicity) can be selected from such libraries by the use of biopanning and other techniques. Because of constraints imposed by the need to transform and subsequently cultivate bacteria, the practical upper limit on peptide library complexity in phage display is said to be around 10¹⁰-10¹¹ [Smothers et al., 2002, Science 298:621-622]. This requirement for passage through E. coli is the result of the relatively complex makeup of the virions of the phages used for phage display, and the consequent necessity that their components be synthesized and assembled in vivo. For example, display of certain peptides is restricted when filamentous phage is used, or not possible, since the fused peptide has to be secreted through the E. coli membranes as part of the phage assembly apparatus.

The present invention employs virus-like phage particles as well as methods for producing these particles in vitro. The resulting phage can be used to conduct phage display in vitro. The methods typically include producing virions in vitro and recovering the virions. As used herein, producing virions “in vitro” refers to producing virions outside of a cell, for instance, in a cell-free system, while producing virions “in vivo” refers to producing virions inside a cell, for instance, an Escherichia coli or Pseudomonas aeruginosa cell.

### Bacteriophages

Properties of single-strand RNA bacteriophages have been described [RNA Bacteriophages, in The Bacteriophages. Calendar, R L, ed. Oxford University Press. 2005]. The known viruses of this group attack bacteria as diverse as E. coli, Pseudomonas and Acinetobacter. Each possesses a highly similar genome organization, replication strategy, and virion structure. In particular, the bacteriophages contain a single-stranded (+)-sense RNA genome, contain maturase, coat and replicase genes, and have small (<300 angstrom) icosahedral capsids. These include but are not limited to MS2, Qb, R17, SP, PP7, GA, M11, MX1, f4, Cb5, Cb12r, Cb23r, 7s and f2 RNA bacteriophages.

For purposes of illustration, the genome of a particularly well-characterized member of the group, called MS2, comprises a single strand of (+)-sense RNA 3569 nucleotides long, encoding only four proteins, two of which are structural components of the virion. The viral particle is comprised of an icosahedral capsid made of 180 copies of coat protein and one molecule of maturase protein together with one molecule of the RNA genome. Coat protein is also a specific RNA binding protein. Assembly may possibly be initiated when coat protein associates with its specific recognition target an RNA hairpin near the 5′-end of the replicase cistron. The virus particle is then liberated into the medium when the cell bursts under the influence of the viral lysis protein. The formation of an infectious virus requires at least three components, namely coat protein, maturase and viral genome RNA, but experiments show that the information required for assembly of the icosahedral capsid shell is contained entirely within coat protein itself. For example, purified coat protein can form capsids in vitro in a process stimulated by the presence of RNA [Beckett et al., 1988, J. Mol. Biol 204: 939-47]. Moreover, coat protein expressed in cells from a plasmid assembles into a virus-like particle in vivo [Peabody, D. S., 1990, J Biol Chem 265: 5684-5689].

### Coat Polypeptide

The coat polypeptide encoded by the coding region is typically at least 120, preferably, at least 125 amino acids in length, and no greater than 135 amino acids in length, preferably, no greater than 130 amino acids in length. It is expected that a coat polypeptide from essentially any single-stranded RNA bacteriophage can be used. Examples of coat polypeptides include but are not limited to the MS2 coat polypeptide, R17 coat polypeptide (see, for example, Genbank Accession No P03612), PRR1 coat polypeptide (see, for example, Genbank Accession No. ABH03627), fr phage coat polypeptide (see, for example, Genbank Accession No. NP_{—}039624), GA coat polypeptide (see, for example, Genbank Accession No. P07234), Qb coat polypeptide (see, for example, Genbank Accession No. P03615), SP coat polypeptide (see, for example, Genbank Accession No P09673), f4 coat polypeptide (see, for example, Genbank accession no. M37979.1 and PP7 coat polypeptide (see, for example, Genbank Accession No PO363 0).

The coat polypeptides useful in the present invention also include those having similarity with one or more of the coat polypeptide sequences disclosed above. The similarity is referred to as structural similarity. Structural similarity may be determined by aligning the residues of the two amino acid sequences (i.e., a candidate amino acid sequence and the amino acid sequence) to optimize the number of identical amino acids along the lengths of their sequences; gaps in either or both sequences are permitted in making the alignment in order to optimize the number of identical amino acids, although the amino acids in each sequence must nonetheless remain in their proper order. A candidate amino acid sequence can be isolated from a single stranded RNA virus, or can be produced using recombinant techniques, or chemically or enzymatically synthesized. Preferably, two amino acid sequences are compared using the BESTFIT algorithm in the GCG package (version 10.2, Madison Wis.), or the Blastp program of the BLAST 2 search algorithm, as described by Tatusova, et al. (FEMS Microbial Lett 1999, 174:247-250), and available at http://www.ncbi.nlm.nih.gov/blast/b12seq/b12.html. Preferably, the default values for all BLAST 2 search parameters are used, including matrix=BLOSUM62; open gap penalty=11, extension gap penalty=1, gap xdropoff=50, expect=10, wordsize=3, and optionally, filter on. In the comparison of two amino acid sequences using the BLAST search algorithm, structural similarity is referred to as “identities.” Preferably, a coat polypeptide also includes polypeptides with an amino acid sequence having at least 80% amino acid identity, at least 85% amino acid identity, at least 90% amino acid identity, or at least 95% amino acid identity to one or more of the amino acid sequences disclosed above. Preferably, a coat polypeptide is active. Whether a coat polypeptide is active can be determined by evaluating the ability of the polypeptide to form a capsid and package a single stranded RNA molecule. Such an evaluation can be done using an in vivo or in vitro system, and such methods are known in the art and routine. Alternatively, a polypeptide may be considered to be structurally similar if it has similar three dimensional structure as the recited coat polypeptide and/or functional activity.

Heterologous peptide sequences inserted into the coat polypeptide or polypeptide may be a random peptide sequence. In a particular embodiment, the random sequence has the sequence Xaaₙ wherein n is at least 4, at least 6, or at least 8 and no greater than 20, no greater than 18, or no greater than 16, and each Xaa is independently a random amino acid. Alternatively, the peptide fragment may possess a known functionality (e.g., antigenicity, immunogenicity). The heterologous sequence may be present at the amino-terminal end of a coat polypeptide, at the carboxy-terminal end of a coat polypeptide, or present elsewhere within the coat polypeptide. Preferably, the heterologous sequence is present at a location in the coat polypeptide such that the insert sequence is expressed on the outer surface of the capsid. In a particular embodiment, the peptide sequence may be inserted into the AB loop regions the above-mentioned coat polypeptides. Examples of such locations include, for instance, insertion of the insert sequence into a coat polypeptide immediately following amino acids 11-17, or amino acids 13-17 of the coatpolypeptide. In a most particular embodiment, the heterologous peptide is inserted at a site corresponding to amino acids 11-17 or particularly 13-17 of MS-2.

Alternatively, the heterologous peptide may be inserted at the N-terminus or C-terminus of the coat polypeptide.

The heterologous peptide may be selected from the group consisting of an HIV peptide, a self antigen, Flag peptide, amino acid sequences derived from the minor capsid protein L2 of human Papillomavirus type 16 (HPV16), the V3 loop of HIV-1 gp120, Bacillus anthracia protective antigen, a receptor, a ligand which binds to a cell surface receptor, a peptide with affinity for either end of a filamentous phage particle specific peptide, a metal binding peptide or a peptide with affinity for the surface of MS2.

In order to determine a corresponding position in a structurally similar coat polypeptide, the amino acid sequence of this structurally similar coat polypeptide is aligned with the sequence of the named coat polypeptide as specified above.

In a particular embodiment, the coat polypeptide is a single-chain dimer containing an upstream and downstream subunit. Each subunit contains a functional coat polypeptide sequence. The heterologous peptide may be inserted ton the upstream and/or downstream subunit at the sites mentioned herein above, e.g., A-B loop region of downstream subunit. In a particular embodiment, the coat polypeptide is a single chain dimer of an MS2 or PP7 coat polypeptide.

### Preparation of Transcription Unit

The transcription unit used in the present invention comprises an expression regulatory region, (e.g., a promoter), a sequence encoding a coat polypeptide and transcription terminator. The RNA polynucleotide may optionally include a coat recognition site (also referred to a “packaging signal”, “translational operator sequence”, “coat recognition site”). Alternatively, the transcription unit may be free of the translational operator sequence. The promoter, coding region, transcription terminator, and, when present, the coat recognition site, are generally operably linked. “Operably linked” or “operably associated with” refer to a juxtaposition wherein the components so described are in a relationship permitting them to function in their intended manner. A regulatory sequence is “operably linked” to, or “operably associated with”, a coding region when it is joined in such a way that expression of the coding region is achieved under conditions compatible with the regulatory sequence. The coat recognition site, when present, may be at any location within the RNA polynucleotide provided it functions in the intended manner.

The invention is not limited by the use of any particular promoter, and a wide variety of promoters are known. The promoter used in the invention can be a constitutive or an inducible promoter. Preferred promoters are able to drive high levels of RNA encoded by me coding region encoding the coat polypeptide Examples of such promoters are known in the art and include, for instance, T7, T3, and SP6 promoters.

The nucleotide sequences of the coding regions encoding coat polypeptides described herein are readily determined. These classes of nucleotide sequences are large but finite, and the nucleotide sequence of each member of the class can be readily determined by one skilled in the art by reference to the standard genetic code.

Furthermore, the coding sequence of an RNA bacteriophage single chain coat polypeptide comprises a site for insertion of a heterologous peptide as well as a coding sequence for the heterologous peptide itself. In a particular embodiment, the site for insertion of the heterologous peptide is a restriction enzyme site.

In a particular embodiment, the coding region encodes a single-chain dimer of the coat polypeptide. In a most particular embodiment, the coding region encodes a modified single chain coat polypeptide dimer, where the modification comprises an insertion of a coding sequence at least four amino acids at the insertion site. The transcription unit may contain a bacterial promoter, such as a lac promoter or it ma contain a bacteriophage promoter, such as a T7 promoter and optionally a T7 transcription terminator. In addition to containing a promoter and a coding region encoding a fusion polypeptide, the RNA polynucleotide typically includes a transcription terminator, and optionally, a coat recognition site. A coat recognition site is a nucleotide sequence that forms a hairpin when present as RNA. This is also referred to in the art as a translational operator, a packaging signal, and an RNA binding site. Without intending to be limiting, this structure is believed to act as the binding site recognized by the translational repressor (e.g., the coat polypeptide), and initiate RNA packaging. The nucleotide sequences of coat recognition sites are known in the art. Other coat recognition sequences have been characterized in the single stranded RNA bacteriophages R17, GA, Qβ, SP, and PP7, and are readily available to the skilled person. Essentially any transcriptional terminator can be used in the RNA polynucleotide, provided it functions with the promoter. Transcriptional terminators are known to the skilled person, readily available, and routinely used.

### Synthesis

As will be described in further detail below, the VLPs used in the present invention may be synthesized in vitro in a coupled cell-free transcription/translation system. Alternatively VLPs could be produced in vivo by introducing transcription units into bacteria, especially if transcription units contain a bacterial promoter.

### Assembly of VLPs Encapsidating Heterologous Peptides

As noted above, the VLPs used in the present invention may encapsidate one or more heterologous peptides. These VLPs may be assembled by performing an in vitro VLP assembly reaction in the presence of the heterologous peptide. Specifically, purified coat protein subunits are obtained from VLPs that have been disaggregated with a denaturant (usually acetic acid). The protein subunits are mixed with the heterologous peptide. In a particular embodiment, the peptide has some affinity for the interior of the VLP and is preferably negatively charged.

Another method involves attaching the heterologous peptide to a synthetic RNA version of the translational operator. During an in vitro assembly reaction the RNA will tightly bind to its recognition site and be efficiently incorporated into the resulting VLP, carrying with it the foreign peptide.

In another embodiment, the peptide is passively diffused into the VLP through pores that naturally exist in the VLP surface. In a particular embodiment, the peptide is small enough to pass through these pores (in MS2 they are about 10 angstroms diameter) and has a high affinity for the interior of the VLP.

### VLP Populations

As noted above, the invention uses VLP populations or libraries. The terms “population” and “libraries” in the instant specification are used interchangeably and are thus deemed to be synonymous. In one particular embodiment, the library may be a random library; in another embodiment, the library is an antigen fragment library, a library of fragments derived from an antigenic polypeptide.

### Random Libraries (Populations)

Oligonucleotides encoding peptides may be prepared. In one particular embodiment, the triplets encoding a particular amino acid have the composition NNS where N is A, G, C or T and S is G or T or alternatively NNY where N is A, G, C, or T and Y is C or T. In order to minimize the presence of stop codons, peptide libraries can be constructed using oligonucleotides synthesized from custom trinucleotide phosphoramidite mixtures (available from Glen Research, Inc.) designed to more accurately reflect natural amino acid compositions and completely lacking stop codons.

### Antigen Fragment Libraries

An alternative strategy takes advantage of the existence of a cloned antigen gene or pathogen genome to create random antigen fragment libraries. The idea is to randomly fragment the gene (e.g. with DNasel) to an appropriate average size (e.g. −30 bp), and to blunt-end ligate the fragments to an appropriate site in coat polypeptide. In a particular embodiment, a restriction site may be inserted into the AB-loop or N-terminus of the coat polypeptide). Only a minority of clones will carry productive inserts, because they shift reading frame, introduce a stop codon, or receive an insert in antisense orientation, any expression vector may in one embodiment contain a marker to pre-select clones with intact coat coding sequences. For example, GalE-strains of E. coli are defective for galactose kinase and accumulate a toxic metabolite when b-galactosidase is expressed in the presence of the galactose analogue, phenyl-b,D-galactoside (PGaI). Subjecting a random antigen-fragment library to selection for translational repressor function in the GalE-strain CSH41 F-containing pRZ5, a plasmid that fuses the MS2 replicase cistron's translational operator to lacZ will eliminate most undesired insertions by enriching the library for those that at least maintain the coat reading-frame.

In certain embodiments, the strategies envisioned herein generally rely on the construction of antigen fragment libraries by one of two general methods: (1) The cloned DNA encoding an antigen or antigens may be randomly generated fragments of an antigen gene and then cloned into RNA phage coat protein. Insertions may usually be made in the coat protein AB-loop, but N-terminal and C-terminal fusions are also anticipated, as are insertions at other as yet unidentified sites in the coat protein sequence. It is important merely that the foreign peptide be displayed on the VLP in a manner that renders it immunogenic. (2) An alternative strategy takes advantage of synthetic oligonucleotide primers to introduce specific foreign peptide insertions, for example by the methods illustrated in FIGS. 1X and 2X. In order to produce a sufficiently complex library, a sufficient number of individual oligonucleotide primers will be required so as to represent all the potential epitopes (even cryptic epitopes) of an antigen or antigens. This can be accomplished for relatively small numbers of sequences by conventional column based oligonucleotide synthesis methods, but new chip based synthesis methods make possible the simultaneous solid-phase synthesis of many thousand of oligonucleotides, which may then be used to produce complex libraries of peptides representing all the potential epitopes of an antigen or multiple antigens.

For the first method the idea is to randomly fragment the gene (e.g. with DNasel) to an appropriate average size (e.g. −30 bp), and to blunt-end ligate the fragments to an appropriate site in coat polypeptide. In a particular embodiment, a restriction site may be inserted into the AB-loop or N-terminus of the coat polypeptide). Only a minority of clones will carry productive inserts, because they shift reading frame, introduce a stop codon, or receive an insert in antisense orientation, Any expression vector may in one embodiment contain a marker to pre-select clones with intact coat coding sequences. For example, GalE-strains of E. coli are defective for galactose kinase and accumulate a toxic metabolite when b-galactosidase is expressed in the presence of the galactose analogue, phenyl-b,D-galactoside (PGal). Subjecting a random antigen-fragment library to selection for translational repressor function in the GalE-strain CSH41 F-containing pRZ5, a plasmid that fuses the MS2 replicase cistron's translational operator to lacZ will eliminate most undesired insertions by enriching the library for those that at least maintain the coat reading-frame.

The second method of antigen fragment library synthesis relies on the synthesis of oligonucleotide primers suitable for insertion of foreign peptide sequences using the insertion schemes shown in FIGS. 1X and 2X. In this case the primers are designed to encode peptide sequences derived from the known amino acid sequences of an antigen or antigens. Since new chip based solid phase synthesis methods allow the simultaneous production of many thousands of oligonucleotide primers, it is possible to synthesize such primers and produce antigen fragment libraries containing many thousands of peptide sequences.

### Synthesis

The synthesis of VLPs is normally directed by plasmids like pDSP1 or pDSP7 (or similar) in E. coli. For the present method VLPs are produced from many different bacterial clones, each synthesizing a VLP with a different antigen peptide. When the antigen sequences are of relatively low complexity—that is, when relatively few sequences (e.g. tens to hundreds of specific peptides), each plasmid can be individually constructed and the identities of the peptides will be known from the beginning of the process all the way through immunization and screening. When many more peptides are involved, however, random fragment libraries will be constructed and the identities of individual cloned peptides may be only determined by sequence analysis after a positive result (i.e. demonstration of its ability to elicit neutralizing antibodies) is obtained. Immunization tests normally require VLPs that have been at least partially purified. To facilitate high-throughput purification of VLPs, the E. coli strain producing them may also contain a second, compatible plasmid such as p6H (FIG. 4). It produces a form of coat protein that contains an affinity tag, in this example a 6×His tag. The tagged coat protein will usually be synthesized in smaller amounts and will coassemble with the coat protein displaying a potential epitope peptide, resulting in the production of mosaic VLPs. Since the tag has affinity for nickel ions, such VLPs can be rapidly purified on nickel affinity media (e.g. NTA-agarose). This enables the simultaneous purification of hundreds or thousands of individual VLPs in sufficient quantities for immunization trials.

In a particular embodiment, the populations of the present invention may be synthesized in a coupled in vitro transcription/translation system using procedures known in the art (see, for example, U.S. Pat. No. 7,008,651 Kramer et al., 1999, Cell-free coupled transcription-translation systems from E. coli, Protein Expression. A Practical Approach, Higgins and Hames (eds.), Oxford University Press). In a particular embodiment, bacteriophage T7 (or a related) RNA polymerase is used to direct the high-level transcription of genes cloned under control of a T7 promoter in systems optimized to efficiently translate the large amounts of RNA thus produced [for examples, see Kim et al., 1996, Eur J Biochem 239: 88 1-886; Jewett et al., 2004, Biotech and Bioeng 86: 19-26].

It is possible in a mixture of templates, particularly in the population of the present invention, different individual coat polypeptides, distinguished by their fusion to different peptides, could presumably package each other's mRNAs, thus destroying the genotype/phenotype linkage needed for effective phage display. Moreover, because each capsid is assembled from multiple subunits, formation of hybrid capsids may occur. Thus, in one preferred embodiment, when preparing the populations or libraries of the present invention, one or more cycles of the transcription/translation reactions be performed in water/oil emulsions [Tawfik et al., 1998, Nat Biotechnol 16: 652-6]. In this now well-established method, individual templates are segregated into the aqueous compartments of a water/oil emulsion. Under appropriate conditions huge numbers of aqueous microdroplets can be formed, each containing on average a single DNA template molecule and the machinery of transcription/translation. Because they are surrounded by oil, these compartments do not communicate with one another. The coat polypeptides synthesized in such droplets should associate specifically with the same mRNAs which encode them, and ought to assemble into capsids displaying only one peptide. After synthesis, the emulsion can be broken and the capsids recovered and subjected to selection. In one particular embodiment, all of the transcription/translation reactions are performed in the water/oil emulsion. In one particular embodiment, only droplets containing only one template per droplet (capsids displaying only one peptide) is isolated. In another embodiment, droplets containing mixed capsids may be isolated (plurality of templates per droplet) in one or more cycles of transcription/translation reactions and subsequently capsids displaying only one peptide (one template per droplet) are isolated.

### Uses of VLPs and VLP Populations

There are a number of possible uses for the VLPs and VLP populations of the present invention. As will be described in further detail below, the VLPs may be used to as immunogenic compositions, particularly vaccines, drug delivery devices, biomedical imaging agents and self-assembling nanodevices. The VLP populations of the present invention may be used to select suitable vaccine candidates.

### Selection Techniques

Techniques for affinity selection in phage display are well developed and are directly applicable to the VLP display system of the present invention. Briefly, an antibody (or antiserum) is allowed to form complexes with the peptides on VLPs in a random sequence or antigen fragment display library. Typically the antibodies will have been labeled with, for example, biotin so that the complexes can be captured by binding to a streptavidin-coated surface, magnetic beads, or other suitable immobilizing medium.

After washing, bound VLPs are eluted, and RNAs are extracted from the affinity-selected population and subjected to reverse transcription and PCR to recover the coat-encoding sequences, which are then recloned and subjected to further rounds of expression and affinity selection until the best-binding variants are obtained. A number of schemes for retrieval of RNA from VLPs are readily imagined. One attractive possibility is to simply capture biotin-mAb-VLP complexes in streptavidin coated PCR tubes, then thermally denature the VLPs and subject their RNA contents directly to RT-PCR. Many obvious alternatives exist and adjustments may be required depending on considerations such as the binding capacities of the various immobilizing media. Once the selected sequences are recovered by RT-PCR it is a simple matter to clone and reintroduce them into E coli, taking care at each stage to preserve the requisite library diversity, which, of course, diminishes with each round of selection. When selection is complete, each clone can be over-expressed to produce a VLP vaccine candidate.

### Immunogenic Compositions

As noted above, the VLPs identified by the screening procedures of the present invention may be used to formulate immunogenic compositions, particularly vaccines. The vaccines should be in a form that is capable of being administered to an animal. Typically, the vaccine comprises a conventional saline or buffered aqueous solution medium in which the composition of the present invention is suspended or dissolved. In this form, the composition of the present invention can be used conveniently to prevent, ameliorate, or otherwise treat a condition or disorder. Upon introduction into a host, the vaccine is able to provoke an immune response including, but not limited to, the production of antibodies and/or cytokines and/or the activation of cytotoxic T cells, antigen. presenting cells, helper T cells, dendritic cells and/or other cellular responses.

Optionally, the vaccine of the present invention additionally includes an adjuvant which can be present in either a minor or major proportion relative to the compound of the present invention. The term “adjuvant” as used herein refers to non-specific stimulators of the immune response or substances that allow generation of a depot in the host which when combined with the vaccine of the present invention provide for an even more enhanced immune response. A variety of adjuvants can be used. Examples include complete and incomplete Freund's adjuvant, aluminum hydroxide and modified muramyl dipeptide.

PP7 is a single-strand RNA bacteriophage of Pseudomonas aeruginosa and a distant relative to coliphages like MS2 and Qβ. We have shown that PP7 coat protein is a specific RNA-binding protein, capable of repressing the translation of sequences fused to the translation initiation region of PP7 replicase. Its RNA binding activity is specific since it represses the translational operator of PP7, but does not repress the operators of the MS2 or Qβ phages. Conditions for the purification of coat protein and for the reconstitution of its RNA binding activity from disaggregated virus-like particles have been established. Its dissociation constant for PP7 operator RNA in vitro was determined to be about 1 nM. Using a genetic system in which coat protein represses translation of a replicase-β-galactosidase fusion protein, amino acid residues important for binding of PP7 RNA were identified. Peabody, et al., Translational repression and specific RNA binding by the coat protein of the Pseudomonas phage PP72001, J. Biol. Chem., June 22; 276(25):22507-13. Epub 2001 Apr. 16.

The coat proteins of several single-strand RNA bacteriophages are known translational repressors. They shut off viral replicase synthesis by binding an RNA hairpin that contains the replicase ribosome binding site. X-ray structure determination of RNA phages shows that homologies evident from comparisons of coat protein amino acid sequences are reflected in their tertiary structures. The coat protein dimer, which is both the repressor and the basic building block of the virus particle, consists of two intertwined monomers that together form a large β-sheet surface upon which the RNA is bound. Each of the coat proteins uses a common structural framework to bind different RNAs, thereby presenting an opportunity to investigate the basis of specific RNA-protein recognition. We have described the RNA binding properties of the coat protein of PP7, an RNA bacteriophage of Pseudomonas aeruginosa whose coat protein shows only 13% amino acid sequence identity to that of MS2.

We have also presented the following findings. 1) The coat protein of PP7 is a translational repressor. 2) An RNA hairpin containing the PP7 replicase translation initiation site is specifically bound by PP7 coat protein both in vivo and in vitro, indicating that this structure represents the translational operator. 3) The RNA binding site resides on the coat protein (3-sheet. A map of this site has been presented. Id.

### PP7 Coat Polypeptide

Examples of PP7 coat polypeptides include but are not limited to the various chains of PP7 Coat Protein Dimer in Complex With Rna Hairpin (e.g. Genbank Accession Nos. 2QUXR; 2QUXO; 2QUX_L; 2QUX_I; 2QUX_F; and 2QUX_C). See also Example 1 herein and Peabody, et al., RNA recognition site of PP7 coat protein, Nucleic Acids Research, 2002, Vol. 30, No. 19 4138-4144.

By way of comparison, the genome of MS2 comprises a single strand of (+)-sense RNA 3569 nucleotides long, encoding only four proteins, two of which are structural components of the virion. The viral particle is comprised of an icosahedral capsid made of 180 copies of coat protein and one molecule of maturase protein together with one molecule of the RNA genome. Coat protein is also a specific RNA binding protein. Assembly may possibly be initiated when coat protein associates with its specific recognition target an RNA hairpin near the 5′-end of the replicase cistron. The virus particle is then liberated into the medium when the cell bursts under the influence of the viral lysis protein. The formation of an infectious virus requires at least three components, namely coat protein, maturase and viral genome RNA, but experiments show that the information required for assembly of the icosahedral capsid shell is contained entirely within coat protein itself. For example, purified coat protein can form capsids in vitro in a process stimulated by the presence of RNA [Beckett et al., 1988, J. Mol. Biol 204: 939-47]. Moreover, coat protein expressed in cells from a plasmid assembles into a virus-like particle in vivo [Peabody, D. S., 1990, J Biol Chem 265: 5684-5689].

Methods for peptide display on the VLPs of RNA bacteriophages, including MS2 and PP7, are described further below.

Briefly, these methods entail the construction of recombinant coat proteins, in which a target peptide is inserted into the coat protein sequence and then displayed in an immunogenic format of the surface of the VLP. In these methods, it was first necessary to identify a form of the RNA phage coat protein, and sites within it, that tolerated insertion of foreign peptides without disruption of its ability to properly fold and assemble into a VLP. We have identified three sites that fulfill this requirement: (1) the N-terminus of coat protein, (2) the so-called AB-loop, and (3) the C-terminus of coat protein. Peptides inserted at these sites are prominently displayed on the surface of the VLP. Unfortunately, the wild-type form of coat protein can be intolerant of peptide insertions, especially in the AB-loop.

For example, the vast majority of AB-loop insertions in the MS2 and PP7 coat proteins (>98%) lead to folding failures. Coat protein normally folds as a dimer, ninety of which assemble into the icosahedral VLP.

To solve the folding problem, we engineered a novel form of coat protein to stabilize it and to render it more tolerant of AB-loop insertions. To do so, we took advantage of the proximity of the N- and C-termini of the two identical polypeptide chains in the dimer. By duplicating the coat protein coding sequence and then fusing the two copies into a single reading frame, we produced a so-called single-chain dimer. This form of the protein is dramatically more stable thermodynamically and its folding is vastly more tolerant of peptides inserted into the AB-loop of the downstream copy of the single-chain dimer. Peptides can also be inserted at the N- or C-termini of the single-chain dimer. Regardless of the insertion site, the resulting VLPs display one peptide per dimer, or ninety peptides per VLP.

As originally described, the RNA phage VLP display technology presents 90 peptides on each VLP since the peptide is inserted at one site in the single-chain dimer, and 90 dimers make up the VLP. The multivalency of these particles may be desirable for certain applications. For example, the high immunogenicity of the particle is related to the high density of the peptides displayed, and is thus a valued property in a vaccine. However, it may be advantageous to display peptides of varying valencies (e.g. a low valency so that they do not induce strong antibody responses).

In preferred embodiments, the methods of the invention are conducted in a high-throughput format.

As explained above, the invention provides a method of identifying a cryptic neutralizing epitope comprising:

(a) defining one or more potential viral cryptic neutralizing epitope-targeting peptide sequences;
(b) determining peptide sequences for each of the defined potential viral cryptic neutralizing epitope-targeting peptide sequences;
(c) reverse-translating the potential viral cryptic neutralizing epitope-targeting peptide sequences to ascertain one or more nucleic acid sequences which encode the potential viral cryptic neutralizing epitope-targeting peptide sequences;
(d) constructing a library of virus-like particles by (i) providing a plurality of a nucleic acid constructs, treating the nucleic acid constructs with a restriction enzyme, and inserting the one or more nucleic acid sequences ascertained in step (c) into the nucleic acid constructs to obtain a population of transcription units (ii) generating virus-like particles by expressing the transcription units in a prokaryote which has been modified to under-express an affinity tag, and (iii) purifying the viral-like particles using the affinity tag and isolating the library;
(e) immunizing a subject with one or more of the viral-like particles; and
(f) identifying one or more viral-like particles that induce a neutralizing antibody response in the subject.

In one embodiment, more than one potential cryptic neutralizing epitope-targeting peptide sequence is defined and each of said sequences corresponds to overlapping peptides from throughout the targeted sequences.

In another embodiment, the virus-like particles comprise a series of peptide units comprising between about 5 to about 20 amino acids, and wherein said series overlaps peptide regions adjacent to the potential viral cryptic neutralizing epitope-targeting peptide sequences by between about 2 to about 10 amino acids.

In still another embodiment, the potential viral cryptic neutralizing epitope-targeting peptide sequence is comprised of between about 100 to about 300 amino acids, and the virus-like particles comprise a series of around 10 to around 1,000 peptide mer units comprising between about 5 to about 20 amino acids.

In still another embodiment, the potential viral cryptic neutralizing epitope-targeting peptide sequence is comprised of between about 100 to about 300 amino acids, and the virus-like particles comprise a series of around 10 to around 50 peptide mer units comprising between about 5 to about 20 amino acids.

In still another embodiment, the sequences of the viral-like particles are known prior to the identification of one or more viral-like particles that induce a neutralizing antibody response in the subject.

In still another embodiment, the sequences of the viral-like particles are known after to the identification of one or more viral-like particles that induce a neutralizing antibody response in the subject.

In still another embodiment, the affinity tag is His6 and the viral-like particles are purified using affinity chromatography.

A variety of peptide tags with different functions and affinities can be used in the invention to facilitate the purification and enrichment of phage. A variety of peptide tag known in the art may be used, such as but not limited to the immunoglobulin constant regions, polyhistidine sequence (Petty, 1996, Metal-chelate affinity chromatography, in Current Protocols in Molecular Biology, Vol. 2, Ed. Ausubel et al., Greene Publish. Assoc. & Wiley Interscience), glutathione S-transferase (GST; Smith, 1993, Methods Mol. Cell. Bio. 4:220-229), the E. coli maltose binding protein (Guar et al., 1987, Gene 67:21-30), and various cellulose binding domains (U.S. Pat. Nos. 5,496,934; 5,202,247; 5,137,819; Tomme et al., 1994, Protein Eng. 7:117-123), protein A, protein G, calmodulin binding peptide (CBP) etc. Other peptide tags are recognized by specific binding partners and thus facilitate isolation by affinity binding to the binding partner, which is preferably immobilized and/or on a solid support. As will be appreciated by those skilled in the art, many methods can be used to obtain the coding region of the above-mentioned peptide tags, including but not limited to, DNA cloning, DNA amplification, and synthetic methods. Some of the peptide tags and reagents for their detection and isolation are available commercially.

In still another embodiment, the method is conducted in a high-throughput format.

Exemplary high-throughput assay systems include, but are not limited to, an Applied Biosystems plate-reader system (using a plate with any number of wells, including, but not limited to, a 96-well plate, a-384 well plate, a 768-well plate, a 1,536-well plate, a 3,456-well plate, a 6,144-well plate, and a plate with 30,000 or more wells), the ABI 7900 Micro Fluidic Card system (using a card with any number of wells, including, but not limited to, a 384-well card), other microfluidic systems that exploit the use of TaqMan probes (including, but not limited to, systems described in WO 04083443 A1, and published U.S. Patent Application Nos. 2003-0138829 A1 and 2003-0008308 A1), other micro card systems (including, but not limited to, WO04067175 A1, and published U.S. Patent Application Nos. 2004-083443 A1, 2004-0110275 A1, and 2004-0121364 A1), the Invader® system (Third Wave Technologies), the OpenArray™ system (Biotrove), systems including integrated fluidic circuits (Fluidigm), and other assay systems known in the art. In certain embodiments, multiple different labels are used in each multiplex amplification reaction in a high-throughput multiplex amplification assay system such that a large number of different target nucleic acid sequences can be analyzed on a single plate or card. In certain embodiments, a high-throughput multiplex amplification assay system is capable of analyzing most of the genes in a genome on a single plate or card. In certain embodiments, a high-throughput multiplex amplification assay system is capable of analyzing all genes in an entire genome on a single plate or card. In certain embodiments, a high-throughput multiplex amplification assay system is capable of analyzing most of the nucleic acids in a transcriptome on a single plate or card. In certain embodiments, a high-throughput multiplex amplification assay system is capable of analyzing all of the nucleic acids in a transcriptome on a single plate or card.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, for example Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2.sup.nd ed., 2001). See U.S. Pat. No. 6,420,108.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention relates to embodiments that include methods for providing information over networks such as the Internet. For example, the components of the system may be interconnected via any suitable means including over a network, e.g. the ELISA plate reader to the processor or computing device. The processor may take the form of a portable processing device that may be carried by an individual user e.g. lap top, and data can be transmitted to or received from any device, such as for example, server, laptop, desktop, PDA, cell phone capable of receiving data, BLACKBERRY™, and the like. In some embodiments of the invention, the system and the processor may be integrated into a single unit. In another example, a wireless device can be used to receive information and forward it to another processor over a telecommunications network, for example, a text or multi-media message.

The functions of the processor need not be carried out on a single processing device. They may, instead be distributed among a plurality of processors, which may be interconnected over a network. Further, the information can be encoded using encryption methods, e.g. SSL, prior to transmitting over a network or remote user. The information required for decoding the captured encoded images taken from test objects may be stored in databases that are accessible to various users over the same or a different network.

In some embodiments, the data is saved to a data storage device and can be accessed through a web site. Authorized users can log onto the web site, upload scanned images, and immediately receive results on their browser. Results can also be stored in a database for future reviews.

In some embodiments, a web-based service may be implemented using standards for interface and data representation, such as SOAP and XML, to enable third parties to connect their information services and software to the data. This approach would enable seamless data request/response flow among diverse platforms and software applications.

In one embodiment, the invention uses nucleic acid construct comprising:

(a) a bacterial or bacteriophage promoter which is operably associated with a coding sequence of either bacteriophage MS2 single chain coat polypeptide dimer or bacteriophage PP7 single chain coat polypeptide dimer, wherein the coat polypeptide dimer coding sequence (1) is modified to define a first restriction site positioned 5′ to that portion of the sequence which defines the coat polypeptide dimer AB loop, and (2) comprises a nucleic acid sequence which encodes a heterologous peptide (putative epitope of a pathogen) and which contains a stop codon which (i) substitutes for that codon which would otherwise encode the coat polypeptide's first amino acid, or (ii) which is positioned at the C-terminus of the single-chain dimer;
(b) a second restriction site positioned 3′ to the coat polypeptide dimer coding sequence; (c) a PCR primer positioned 3′ to the second restriction site;
(d) an antibiotic repressor which is operably associated with the promoter, and
(e) a replication origin for replication in a prokaryotic.

In the construct described above, a first primer can be positioned 5′ to the first restriction site and a second primer can be positioned 3′ to the second restriction site and 5′ to the PCR primer.

In some embodiments, the bacterial or bacteriophage promoter is a T7 promoter, the first restriction site is either a SalI and KpnI restriction site, the second restriction site is a BamHI site, the PCR primer is TP7, the antibiotic repressor is a kanamycin resistance gene, and the replication origin is colE1 ori.

In other embodiments, the construct further comprises a transcription terminator positioned 5′ to the second restriction site, or the construct optionally comprises a transcription terminator positioned 5′ to the second restriction site.

In another embodiment, the nucleic acid construct comprises:

(a) a bacterial or bacteriophage promoter which is operably associated with a coding sequence of either bacteriophage MS2 single chain coat polypeptide dimer or bacteriophage PP7 single chain coat polypeptide dimer, wherein the coat polypeptide dimer coding sequence (1) is modified to define a first restriction site positioned 5′ to that portion of the sequence which defines the coat polypeptide dimer AB loop, and (2) comprises a nucleic acid sequence which encodes a heterologous peptide (putative epitope of a pathogen) and which contains a stop codon which (i) substitutes for that codon which would otherwise encode the coat polypeptide's first amino acid, or (ii) which is positioned at the C-terminus of the single-chain dimer;
(b) a restriction site positioned 3′ to the coat polypeptide dimer coding sequence;
(c) a PCR primer positioned 3′ to the second restriction site;
(d) a repressor to resistance to a first antibiotic, wherein the repressor is operably associated with the promoter;
(e) a helper phage gene modified to contain a gene conferring resistance to a second antibiotic, and
(f) a replication origin for replication in a prokaryotic cell.

In the embodiment described above, the codon sequence may contain the maximum possible number of silent nucleotide substitutions, the repressor to resistance to a first antibiotic may be a kanamycin resistance gene and the helper phage gene may be modified to contain a gene conferring resistance to chloramphenicol, and the construct may comprise a transcription terminator positioned 5′ to the second restriction site.

In one embodiment, the bacterial or bacteriophage promoter is a T7 promoter, the RNA bacteriophage single chain coat polypeptide dimer is a MS2 or PP7 coat protein single chain dimer, the codon sequence contains the maximum possible number of silent nucleotide substitutions, the restriction site is a BamHI site, the PCR primer is TP7, the repressor to resistance to a first antibiotic is a kanamycin resistance gene, the helper phage gene is modified to contain a gene conferring resistance to chloramphenicol, and the replication origin is colE1 ori.

The nucleic acid constructs of the invention may use an amber stop codon, and may optionally comprises a transcription terminator positioned 5′ to the second restriction site. In some of these embodiments, the repressor to resistance to a first antibiotic is a kanamycin resistance gene and the helper phage gene is modified to contain a gene conferring resistance to chloramphenicol.

In some embodiments, the prokaryote is an E. coli strain; the plasmid is pNMsupA; and the suppressor tRNA gene is an alanine-inserting suppressor tRNA gene which is expressed under the control of a lac promoter.

The promoter whose activity can be modulated can be proB. In one embodiment, valency of the virus-like particles is adjusted by controlling the expression of the suppressor tRNA gene and the plasmid which comprises a suppressor tRNA gene is pNMsupA.

Techniques that are well-known to those of ordinary skill in the art can be used in the various steps of the methods described above.

For example, genome engineering technologies now enable reengineering genomes from the nucleotide to the megabase scale, including the identification and replacement of stop codons. Isaacs, et al., Precise manipulation of chromosomes in vivo enables genome-wide codon replacement, Science. 2011 Jul. 15; 333 (6040):348-53.

Methods of making suppressor tRNA that comprises a unique three base codon of natural and/or unnatural bases, or is a nonsense codon, a rare codon, an unnatural codon, a codon comprising at least 4 bases, an amber codon, an ochre codon, or an opal stop codon, are disclosed in U.S. Pat. No. 8,012,739 and may be used in the methods described herein. United States Patent Application Document No. 20110166868 also provides useful ways of identifying and employing suppressor tRNA's.

TLR-specific polypeptides can be identified using techniques such as those disclosed in United States Patent Application Document No. 20090068224.

“CR2 ligand” refers to any molecule that binds to a naturally occurring CR2 protein, which include, but are not limited to, C3d, iC3b, C3dg, C3d, and cell-bound fragments of C3b that bind to the two N-terminal SCR domains of CR2. The CR2-FH molecule may, for example, bind to a CR2 ligand with a binding affinity that is about any of 10%, 20%, 30%, 40%, 50%, 60%, 70%, 80%, 90%, or 100% of the CR2 protein. Binding affinity can be determined by any method known in the art, including for example, surface plasmon resonance, calorimetry titration, ELISA, and flow cytometry. In some embodiments, the CR2 ligand has one or more of the following properties of CR2: (1) binding to C3d, (2) binding to iC3b, (3) binding to C3dg, (4) binding to C3d, and (5) binding to cell-bound fragment(s) of C3b that bind to the two N-terminal SCR domains of CR2.

### Plasmid Background Information

Two types of plasmid vector have been constructed for purposes of library construction in MS2 and PP7 VLPs. The general features of pDSP1 and pDSP62, plasmids for expression of MS2 VLPs, are described in some detail below. Two other plasmids (pDSP7 and pDSP72) were constructed for expression of PP7 VLPs. Their features are highly similar to those of pDSP1 and pDSP62. For simplicity only the MS2 VLP producers are described here. Plasmid pDSP1 was constructed for convenient cloning of PCR-generated—or other double-stranded DNA—fragments into the AB-loop of the downstream copy of a coat protein single chain dimer. The second is called pDSP62 and was constructed specifically for introduction of peptide sequences at virtually any position in the single-chain dimer (usually the AB-loop) by the site-directed mutagenesis method of Kunkel et al. (1). The general features of the plasmids are presented below.

The adaption of these plasmids for use in accordance with the invention claimed herein (e.g. the insertion of a stop codon which substitutes for that codon which would otherwise encode the coat polypeptide's first amino acid) has been explained in detail above and is further exemplified hereinafter.

pDSP1—a Plasmid Expressing a Single-Chain Dimer with Convenient Cloning Sites for Insertion in the AB-Loop.

The plasmid pDSP1 contains the T7 transcription signals of pET3d and the kanamycin resistance and replication origin of pET9d. [Information regarding pET3d and pET9d may be found at the New England Biolabs vector database, https://www.lablife.org/ct?f=v&a=listvecinfo). It expresses the coding sequence of the MS2 single-chain coat protein dimer (2), modified to contain unique SalI and KpnI restriction sites. This facilitates simple cloning of foreign sequences into the AB-loop. To make these sites unique, it was necessary to destroy other Sail and KpnI sites in the vector and in the upstream coat sequence.

The MS2 coat sequence in the vicinity of the AB-loop insertion site for pDSP1 is shown below. Note the presence of SalI and KpnI sites.

. . . 6 7 8 9 10 11 12 13 14 15 16 17 18 19 20 21 22 . . .
. . . GlnPheValLeuValAspAsnGlyGlyThrGlyAspValThrValAlaPro . . .
. . . CAGTTCGTTCTCGTCGACAATGGCGGTACCGGCGACGTGACTGTCGCCCA . . .
SalI KpnI

Shown below is an example of a random 7-mer library in pDSP1, with the random sequence inserted in the so-called 13/16 mode.

. . . 6 7 8 9 10 11 12 13 16 17 18 19 20 21 22 . . .
. . . GlnPheValLeuValAspAsnGly x x x x x x x GlyAspValThrValAlaPro . . .
. . . CAGTTCGTTCTCGTCGACAATGGCNNSNNSNNSNNSNNSNNSNNSGGCGACGTGACTGTCGCCCCA . . .
SalI

The 5′-PCR primer shown below would be used with the 3′ primer 12013 to generate a fragment of the coat protein coding sequence with the random sequence inserted between amino acids 13 and 16. N=A, C, G, or T and S=G or C. After digestion with SalI and BamHI, the fragment would be inserted between SalI and BamHI of pDSP1. Shown below is an example of a 5′-primer that could be used to generate such a library:

5′-CGCGTCGACAATGGC(NNS)₇GGCGACGTGACTGTCGCCCCA-3′

With pDSP1, random-sequence peptide libraries are usually constructed by cloning into the AB-loop a PCR fragment generated using a monomeric coat protein sequence as template (e.g. pMCT). A synthetic oligonucleotide 5′-primer is designed to attach a SalI (or KpnI) site and a sequence of random codons (e.g. 6-10 copies of NNY) to a site just upstream of the AB-loop. A 3′-primer anneals to sequences in the plasmid vector just downstream of BamHI. The resulting PCR product is digested with SalI (or KpnI) and BamHI and cloned at the corresponding sites of pDSP1. This results in insertion of peptides into the AB-loop, the exact site of insertion depending on the specific design of the 5′-primer. For most insertions use of the SalI site is preferred as it affords more flexibility that KpnI in selection of the insertion site. With these methods it is relatively straightforward to produce peptide VLP libraries with up to 10⁸-10⁹ individual members.

pDSP62—a Plasmid Suitable for Library Construction Using Efficient Site-Directed Mutagenesis Methods.

Introduction of an M13 Origin of Replication.

Methods for library production like that described above for pDSP1, are difficult to scale up, because it is inconvenient to purify DNA restriction fragments in the necessary quantities. Moreover, during ligation reactions some of the DNA is inevitably diverted into useless side-products, reducing the yield of the desired plasmid. The construction of complex libraries would be facilitated by methods that efficiently produce larger yields of the correct recombinant DNA than are found in a typical ligation reaction. Specifically, we want to make use of a variation of an old method for site-directed mutagenesis, which has been used already by others to produce peptide libraries on filamentous phage in the 10¹¹ complexity range (1,3). The method is applied to single-stranded circular DNAs produced from a particular kind of plasmid (also know as a phagemid) that contains an M13 origin of replication. Infection with an M13 helper phage (e.g. M13K07) of a dut⁻, ung⁻ strain (e.g. BW313) containing the plasmid results in facile production of dUTP-substituted single-stranded DNA. In the actual mutagenesis reaction, a mismatched oligonucleotide primer is annealed to the single-stranded DNA template and is elongated using a DNA polymerase (e.g. that of T7 phage). The DNA is ligated to produce closed circular DNA, and introduced by transformation into and ung⁺ strain, where the mutant strand is preferentially replicated. Experience in the production of peptide-VLP libraries indicates that typically about 90% of the transformants contain the desired peptide insertions. The primer extension mutagenesis reaction can be conducted on relatively large quantities of DNA (e.g. 20 ug), enough to readily generate on the order of 10¹¹ individual recombinants by electroporation.

To facilitate the production of single-stranded DNA, we introduced an M13 origin of replication into pDSP1. To do so, the M13 origin found in pUC119 was amplified by PCR and cloned at a unique AlwNI site in pDSP1. This plasmid, called pDSP1-IG, is the progenitor to pDSP62. Because it is only an intermediate to the construction of pDSP62 I, its sequence is not shown.

Targeting Insertions to Only One Half of the Single-Chain Dimer Through the Use of a Synthetic “Codon-Juggled” Coat Gene.

The desire to use primer-extension mutagenesis for efficient peptide library construction introduced a new complication. Our display method relies on the ability to specifically introduce foreign peptides into only one of the two AB-loops of the single-chain dimer. Using the single-chain dimer sequence present in pDSP1, the mutagenic primer would anneal to sequences in both halves, resulting in double insertions. But we already know that insertions in both AB-loops result in a high frequency of protein folding failures. Moreover, even if the insertions were tolerated, an site-directed mutagenesis that failed to target only one half of the single-chain dimer would result in the display of two different peptides on each VLPs. For these reasons, we synthesized a “codon-juggled” version of coat protein and exchanged it for the normal upstream half of the single-chain dimer. The codon-juggled sequence contains the maximum possible number of silent nucleotide substitutions, and thus produces a polypeptide having the wild-type coat protein amino acid sequence. However, the presence of numerous mutations makes the juggled sequence incapable of efficiently annealing to the mutagenic oligonucleotide, and therefore the mutagenic primer is specifically directed to the downstream AB-loop sequence.

A Chloramphenicol-Resistant M13 Helper Phage for Single-Strand pDSP62 Production.

Plasmid pDSP62 confers resistance to kanamycin. The helper phages (e.g. M13KO7) usually used for production of single stranded phagemid DNA also confer kanamycin resistance, and are therefore unsuitable for use with the plasmids described here. For this reason we constructed M13CM1, a chloramphenicol resistant derivative of M13KO7. In the presence of kanamycin (selects for pDSP62 maintenance) and chloramphenicol (selects for helper phage), cells produce large quantities of single-stranded plasmid DNA after infection with M13 CM1. Using these single-stranded templates and the method of Kunkel et al. (1), random sequence peptide libraries have been readily produced that contain more than 10¹⁰ individual members for [NNS]₆, [NNS]₇, [NNS]₈ and [NNS]₁₀. Significantly higher complexities are possible with scale-up.

### REFERENCES FOR PLASMID BACKGROUND INFORMATION

1. Kunkel, T. A., Bebenek, K., and Mcclary, J. (1991) Methods in Enzymology 204, 125-139
2. Peabody, D. S., and Lim, F. (1996) Nucleic Acids Res 24, 2352-2359
3. Sidhu, S. S., Lowman, H. B., Cunningham, B. C., and Wells, J. A. (2000) Methods Enzymol 328, 333-363
4. Chang, A. C., and Cohen, S, N. (1978) J Bacteriol 134, 1141-1156

### Further Description of the Methods of the Invention:

To better understand the claimed methods, each of their steps can be considered in terms of the following steps, which are presented as a purely exemplary embodiment.

Step 1. Identify Open Reading Frames in a Target Antigen Sequence and Make Antigen Fragment VLP Libraries.

[Note that above we described two general approaches to the production of antigen fragment libraries. One directly fragments DNA encoding the antigen(s) and then inserts those fragments into the coat protein gene in such a manner that the foreign (i.e. antigen) peptides are displayed on the surface of the coat protein VLP. Although certainly applicable here, that method suffers the disadvantage of random insert orientation (i.e. forward and reverse), and frequent disruption of the coat protein reading frame (since it is difficult with random fragmentation to ensure the insertion of fragments whose lengths are multiples of 3 nucleotides). For these reasons we favor approaches such as those described above and now below that utilize DNA synthesis to produce the antigen fragments.}

The first step is to define the target sequences. These can be as simple as a single protein antigen or, in principle, as complex as all the proteins encoded in a genome. Next decide on the sizes of the peptides to be displayed on VLP's. To minimize arbitrary interruption of epitopes, consider utilizing overlapping peptides from throughout the targeted sequences. Next, list all the overlapping peptide-encoding sequences needed to ensure complete coverage of the target open reading frame(s), and reverse translate them into oligonucleotide primers. For example, make primers to insert into VLP's a series of peptide 10-mers with a 5-amino acid overlaps that represent the entire sequence of the targeted antigen(s). During oligonucleotide design, it may be desirable to mutate any of the codons that appear rarely in E. coli to more abundant ones and then send the list to the synthesizer. In one system, each synthetic peptide-encoding sequence could be flanked by 18-mer oligonucleotides complementary to sequences flanking the site of insertion in MS2 or PP7. These primers would then be used for second-strand synthesis on single-stranded pDSP62 or pDSP7 templates (see FIG. 2) and transformation of E. coli or as PCR primers in the scheme illustrated in FIG. 1.

For a typical protein of, say, 200 amino acids, thirty-nine 10mers with 5 amino acid overlaps are required to represent the whole sequence. In such a case, the number of individual VLP's needed is so small that each VLP is constructed individually, so its identity is known from the beginning and is tracked throughout the process. When the library is much more diverse, however, it is simpler to construct the VLP's as complex mixtures, from which individual members are randomly screened and their identities are determined only at the end of the process. As an example of intermediate complexity, consider Hepatitis C Virus. Its genome encodes a single polyprotein with a total of about 3,000 amino acids, which is processed proteolytically to produce all the individual viral proteins. The complete amino acid sequence can be represented by about 600 10mer peptides with 5-amino acid overlaps. Or consider a bacterial genome with, say, 4,000 proteins, each 200 amino acids long, on average. Around 156,000 10mer peptides with 5-amino acid overlaps are needed to obtain complete coverage of all these sequences. Of course, in the case of bacterial pathogens, one would most likely choose to target known or bioinformatically identified cell-surface antigens, thus greatly restricting the required library complexity.

Note that the preferred method of primer synthesis depends on the number of sequences to be synthesized. For libraries representing only one or a few antigen proteins, the synthesis can be performed using conventional, column-based methods. However, truly large numbers (hundreds to millions) will require microarray methods, which now make possible the synthesis of hundreds, thousands, or hundreds of thousands of specific oligonucleotide primer sequences.

Step 2. Make a Library of VLP's Expressing the Peptides.

The oligonucleotides synthesized as described above are used to create VLP libraries using our plasmid vectors and methods described herein. Two kinds of libraries can be made, depending on the size of the target antigens (i.e. the number of clones needed to cover the antigen sequence). For example, when the target is relatively small and can be covered with say 10-100 different recombinants, the oligonucleotides can be made using conventional synthesis methods and each recombinant VLP can be individually constructed and purified in step 4 below. In such cases, each primer can be used individually to produce a VLP whose identity will be known from the start and tracked throughout the entire process. When the target is large, for example all the ORFs of a bacterial genome, a mixed library will be constructed consisting of many individual members. In such a library the sequence identities of individual VLP clones are only determined by sequence analysis after screening is complete (see steps 4 an 5 below).

Step 3. Synthesize and Purify the VLP Library.

Use of VLPs for immunization requires that they be purified first. Our usual methods depend on purification by gel filtration chromatography and, although straightforward for a few VLP's, they are too laborious for purification of large numbers. We can solve this problem by introducing an affinity tag (e.g. His6) in the VLP. To accomplish this, the VLP will be synthesized in a bacterial strain that also produces a lower level of His-tagged coat protein, from, for example, a plasmid such as p6H, which contains an AB-loop insertion in the MS2 coat protein of six histidines. The general structure of the plasmid and the sequence of the His6-tag insertion is shown in FIG. 3. The resulting mosaic particles (with, say, 10% of coat proteins displaying the tag) are rapidly and easily purified by affinity chromatography on an Ni²⁺NTA-resin. This will be straightforward to automate, making it easy to purify 100's to 1,000's of individual VLPs at a time. It should be noted that the 6×His tag is only one of several that could be employed.

Step 4. Immunization.

This is not much of a problem with small numbers (e.g. 10's to 100's) of VLPs, since each animal can be immunized with a cocktail of perhaps 10 different VLPs at a time, and since, although laborious, it is possible to handle 100's of animals. For larger numbers (e.g. 1000's) it will be necessary to utilize an in vitro immune system so as to produce antibodies in a high-throughput manner.

Step 5. Identify VLPs that Induced a Neutralizing Antibody Response in Step 5.

Neutralization assays range in complexity depending on the available antigen-specific technology. In many instances, for example, simple plaque assays can determine whether a neutralizing response against a viral antigen has been elicited. In more complicated cases the search can first be narrowed by screening antisera by ELISA for sample reaction with the antigen. Any reactive antibodies thus obtained can then be tested for neutralization activity in the more laborious in vitro or in vivo assays.

Techniques that are well-known to those of ordinary skill in the art can be used in the various steps of the methods described above.

For example, conventional reverse translation can employ the following approach. A purified peptide or protein is sequenced using an automated amino acid sequencing machine. Following sequencing, the identity and order of the amino acids are read. From the sequence, an oligonucleotide is synthesized using a second instrument, an oligonucleotide synthesizer. Oligonucleotides may also be synthesized manually. From the prepared oligonucleotide, the full-length polynucleotide can be cloned. From the full-length polynucleotide, the protein can be produced. Alternatively, automated reverse translation techniques disclosed in U.S. Pat. No. 7,820,786 can be used.

Oligonucleotide primer sequences can be determined using one or more of the techniques disclosed in United States Patent Application Document No. 20110021383.

Affinity tag methodologies described in United States Patent Application Document No. 20110218379 can be employed to facilitate purification of the VLP libraries.

Exemplary high-throughput immunization techniques and processes for identifying immune responses are disclosed in Tang, et al., A novel high-throughput neutralization assay for supporting clinical evaluations of human cytomegalovirus vaccines, Vaccine. 2011 Sep. 7 and Reddy, et al., Systems analysis of adaptive immunity by utilization of high-throughput technologies, Curr Opin Biotechnol. 2011 August; 22(4):584-9. Epub 2011 May 12.

The invention is described further in the following examples, which are illustrative and in no way limiting.

### Example 1

### Novel Peptide Display and Affinity Selection Platform

Conventional phage display mostly uses filamentous phages like M13, and while it is a powerful system for epitope mapping, it suffers from the drawback of limited immunogenicity of the peptides it displays. This is the consequence of a quirk of filamentous phage molecular biology that makes it difficult to display foreign peptides are the high densities that best favor high immunogenicity. To test a peptide's value as a potential epitope vaccine, it is usually necessary first to synthesize it chemically and then conjugate it to a more immunogenic carrier in an expensive, time consuming and inconvenient process. Even worse, some epitopes lose affinity for the selecting target (and ability to elicit the desired antibody response) when moved from the structural environment they experienced during affinity selection. We have developed a new peptide display and affinity selection platform that integrates the epitope discovery and immunization functions into a single virus-like particle derived from RNA bacteriophage MS2. The system has already demonstrated its utility for finding linear epitopes, and, in experiments now underway, is also showing promise for discovery of peptide mimics of protein conformational epitopes.

This invention specifies the use of RNA phage VLP-based phage display in two broad ways toward identification of epitopes of viral, bacterial and protozoan pathogens with the goal of (1) identifying epitopes of particular pathogenic significance, such as those epitopes that are exposed only in cases of secondary dengue virus (DENY) infection; (2) identification of epitopes of potential significance in vaccine development; and (3) identifying epitopes of diagnostic significance. We already described the development of peptide display platforms based on RNA phages MS2 and PP7 with insertions in either the AB-loops or at the N- or C-termini of the phage coat proteins. The use of two general types of library is anticipated:

1. Antigen Fragment Libraries. Preparation of a library of phage in which an “antigen-fragment library” of overlapping peptides that span the entire proteome of a viral pathogen or the entire surface-expressed proteome of a bacterial or protozoon pathogen has been inserted into an antigenically-favored and polyvalent part of the phage (eg MS2 or PP7) particle (“antigen fragment library”). The peptide length and extent of overlap may vary depending on a given specific application. The relatively small size of most viral genomes allows the production of oligonucleotides that scan the entire genome with a series of peptides, progressing through the sequence in a series of small steps. For example, scanning the entire genome of, say, 12 kb with 10-mers at 1-amino acid increments (i.e. with 9-amino acid overlaps) requires only about 4,000 primers, a number well within the capabilities of the new micro-chip-based oligonucleotide synthesis methods. An antigen fragment library has the advantage of yielding a complete representation of the peptidome of either a pathogen, or of the most relevant (e.g. surface-expressed) portions of a pathogen's (or other antigen target's) peptidome. No extraneous, non-pathogen sequences are present. However, it is essentially restricted to the display of linear epitopes, at least when relatively small peptides are displayed.

Some pathogens, of course, have much larger genomes than a typical virus. Existing oligonucleotide synthesis methods allow for the production of around 55,000 independent sequences on a single chip, and this number is bound to increase as the technology advances. This means that libraries containing perhaps a half-million to a million individual antigen fragments are already possible using existing methods. Increases in the average size of displayed peptides, or decreases in the extent of their overlap reduce the number of individual sequences necessary to represent the entire peptidome of an organism, thus increasing the size of the peptidome the method can cover. Even so in many instances it will make sense to target only those proteins that are expressed on a pathogen's surface, or are otherwise implicated as having antigenic significance.

It should also be noted that other methods for construction of antigen-fragment libraries exist. They typically rely on actual random fragmentation of DNA representing the genome (or antigen) of interest, followed by insertion into the display site. These methods can be used, of course, but for most applications they must be considered inferior to the one described above.

2. Random Sequence Libraries. This entails preparation of a library similar to (1) above, except that random sequence peptides are inserted into the appropriate display site on the appropriate RNA phage coat protein gene. Random sequence libraries will usually contain much higher sequence complexity than the antigen fragment libraries, but have the advantage that they may contain peptide mimetics of protein conformational epitopes or of carbohydrate epitopes that are normally inaccessible by the antigen fragment approach, especially when the antigen fragments are relatively small.

### Affinity-Selection and Epitope Identification.

We previously demonstrated the ability of affinity selection (e.g. biopanning) to find defined peptide epitopes in complex random sequence peptide libraries using the RNA phage VLP display system on monoclonal antibody targets. In the present application antigen-fragment or random sequence libraries are subjected to affinity selection using human or animal polyclonal antisera known to contain antibodies directed to epitopes of interest, even if their identities are not yet known. For example, serum from a Dengue Virus-infected individual will normally contain antibodies able to neutralize the infecting virus. Affinity selection using such sera, and comparing them to the peptides recognized by uninfected sera, allows us to identify those peptides in the library specifically recognized by those antibodies. Among these will be some that are bound by virus-neutralizing antibodies.

The affinity-selected peptides are identified by DNA sequence analysis. The use of a highly parallel, high-throughput sequence method (i.e. deep sequencing) of the VLPs present after one or more cycles of selection on an antigen fragment library allows an essentially complete characterization of all the epitopes of the agent in question—yielding, in effect, a high resolution epitope map of the antigen(s). Similar analysis of selections using random sequence libraries confirms the identities of linear epitopes, and also identifies potential mimics of conformational and carbohydrate epitopes.

Deep sequencing identifies the individual members of a complex population, but does not associate them with a given clone. It is a simple matter to synthesize an oligonucleotide representing each peptide sequence and then introduce it into a VLP expression plasmid. Purification of the VLPs by conventional methods, on the other hand, is too cumbersome to conduct in large numbers on a reasonable time scale. We have recently developed a simple high-throughput approach to VLP purification. From a second, compatible plasmid called pAC3dH6 we express a relatively small amount (about 10-15% of total coat protein) of a his-tagged coat protein. It co-assembles with the larger amount of an epitope-displaying coat protein to produce mosaic particles that are readily purified on nickel-affinity columns. Since VLPs from our plasmids are produced at about 20 mg per liter of culture, we should be able to purify amounts sufficient for our needs from 5-10 ml cultures. It is unlikely that a small percentage of His-displaying coat protein will substantially effect antibody titers; we have recently shown that a 50% reduction in epitope density has minimal effects (unpublished data).

### Immunologic Screening of VLP Selectants for Display of Neutralizing Epitopes.

To distinguish those epitopes able to elicit a neutralizing response it will be necessary to immunize animals with purified VLPs and test the resulting antisera for the ability to inhibit infection or some other essential activity of the relevant agent/antigen. The emergence of in vitro immune systems may make this process more efficient and allow higher throughput, but at present at least hundreds of epitopes may be screened using animals (e.g. mice). When large numbers are involved, it is possible to pool as many as 10 VLPs (or more) for immunization of each animal, and these are later sorted out by immunization with individual VLPs from pools that elicit a neutralization response.

The list of pathogens to which this method might be applied includes any virus, bacterium or parasite for which the genomic sequence is known and which infects humans or any animal. The viral list includes dengue virus; yellow fever virus; West Nile virus; Japanese encephalitis virus; HIV; HTLV-I, Bunyaviridae viruses including the hantaviruses, Crimean-Congo hemorrhagic fever, Rift Valley fever virus, and fever and severe fever and thrombocytopenia virus; arenaviruses including all agents of South American hemorrhagic fever, Lassa virus and lymphocytic choriomeningitis virus; filoviruses including Ebola and Marburg viruses; paramyxoviruses including morbilliviruses, henipaviruses, respiroviruses including RSV and metapneumovirus and rubellaviruses; Alphaviruses including Chikungunya, O'nyung-nyung, Semliki Forest, Ross River, Sindbis, eastern, western and Venezuelan equine encephalitis; picornaviruses; papillomaviruses including HPV; herpesviruses including HSV-1/2, EBV, CMV, VZV, HHV-6, 7, and 8; polyomaviruses including SV40, JC and BK viruses; poxviruses including variola and vaccinia viruses. The bacterial pathogens include any human pathogen such as Staphylococcus spp; Streptococcus spp; E. coli and other pathogenic coliforms. Parasitic pathogens include malaria (Plasmodium spp). In no sense do we consider this technology limited to the above illustrative list, nor is it restricted to pathogens of humans. Furthermore, the technology can be applied to any protein for which it is advantageous to understand or exploit the knowledge of its antigenicity (or allergenicity, as assessed by studying specific IgE responses) in connection with its introduction into humans or animals. Thus, the method could be used to characterize the immune response to coagulation factor VIII in a hemophiliac, the epitome of pathogenic or cellular forms of the proteins (PrPs) associated with Creutzfeld-Jacob disease or scrapie, or the immune responses of vaccines to the agent used as immunogen. The method could quickly identify the antigenic portion(s) of any vaccine or therapeutic biologic, for example, as part of a pre-clinical screen for the antigenicity or allergenicity of such proteins, or in evaluating the antigenicity or allergenicity of products that elicit immune or allergic responses in an individual or group of patients or animal subjects.

The more detailed description that follows is developed using the dengue viruses as model, but all of the principles could also be applied to entire virus, bacterial or parasitic genomes, or, for bacteria or parasites, could be simplified by considering only the part of the genome that encodes for protein determinants that are known to be, or are predicted to be, expressed on the surface of the pathogen's cells at one or another stage of infection.

### Application to Dengue Viruses (DENV).

Dengue viruses (Flaviviridae: Flavivirus: dengue virus) comprise four separate species of flaviviruses, designated DENY-1 through 4. Approximately 2.5 billion people in 100 countries, largely in the tropics, are at risk for this mosquito-borne complex of viruses, which infects 50-100 million persons per year. This makes it the most widespread zoonotic viral infection in the world.

The first (primary) infection of a person by a dengue virus can be inapparent or cause dengue fever (aka breakbone fever), a very painful and debilitating but usually self-limiting acute fever and exanthem, accompanied by severe muscle and bone pain. Such infection confers lifelong immunity but only against the virus that caused the original infection, such as DENV-1. Subsequently, it is possible to acquire a second infection with another DENV type (2, 3, or 4 in the example), and in many cases, such secondary infections are significantly more severe and life-threatening compared to the primary infection. A major mechanism for such enhanced pathogenicity with secondary DENV is the phenomenon known as antibody-dependent enhancement (ADE). In ADE, non-neutralizing or slightly-neutralizing antibodies, originally raised against the virus that caused the primary infection (eg DENV-1), are able to cross-react with the virus that caused the secondary infection (eg, DENV-2, 3, or 4) and cause that virus to be taken into macrophages or dendritic cells as a consequence of the antibody's engagement by the Fc receptor. Such alternate means of entry are thought to confer replicative advantage to the virus causing the secondary infection in vivo, while possibly allowing the virus to avoid immune recognition.

Many laboratories have studied the epitopes recognized in the course of primary infection by DENV as well as those recognized during secondary infections, but prior art does not specify any highly-sensitive, high-throughput and generic way to systematically identify such epitopes. The present invention changes this situation dramatically in that it offers, for the first time, the possibility to systematically survey all or virtually all linear epitopes that are recognized by people who have been infected with DENV. In particular, because the MS2 phage display system displays 90 copies of the DENV-derived or random-amino acid peptide per particle, it serves as an extraordinarily efficient platform for recognition by the mammalian immune system, such that even vanishingly small quantities or low-affinity antibodies directed against such peptides can be detected.

The DENV peptides encoded and expressed in the MS2 coat protein in the antigen-fragment libraries are derived from the published sequences of the four DENV serotypes and span the entire coding sequence of each of the four viruses. To assure that no linear epitopes can be missed, each member of the DNA library encoding the peptides is offset from the preceding peptide by only a single residue, ie, they overlap by 9 of the 10 residues in the case of a library of 10-mers. For a genome of the size of DENV, about 10 kilobases, encoding some 3400-aa as a single polyprotein and 10-mer peptides, such a library will have about 3400 members. [It is important to note that this is only an example. The peptide length and extent of overlap may be varied according to requirements. With very large proteomes, for example, the number of individual library recombinants needed to obtain complete coverage can be greatly reduced through the use of longer peptides and/or smaller overlaps.]

The present invention specifies that after insertion of the ˜3400 30-nt DENV-derived DNA fragments into the appropriate site of the RNA phage (eg, MS2) coat protein gene, the phage (virus-like particle) library is prepared when the phage coat proteins bearing the DENV-specific inserts is transformed into E. coli and the particles are produced in bulk culture. The library, eg that for DENV-1, is then exposed to the polyclonal antiserum from a patient with DENV-1 fever in the solid phase, and the phage VLP that are bound by the polyclonal antiserum are selected for their ability to bind. Multiple rounds of such selection can be carried out under conditions in which the valency of the peptide in the phage can be maintained as high (180-copies/VLP) to maximize the efficiency with which epitopes are recognized, or can be decreased by means described in our prior art such that only those VLPs that are recognized with high affinity are selected. In net however, with appropriate controls such as using antisera from uninfected persons, it is possible to systematically survey the DENV proteome for any and all epitopes that are recognized even at low affinity and that are present in the serum at low concentrations.

The consequence of such capacity are manifold but include the following:

### A. Antigen-Fragment Library

1. By systematically surveying the DENV genome for hundreds to potentially hundreds of thousands of specificities, it is possible to array the RNA phage VLPs that react to detect and characterize the performance of these thousands of epitopes simultaneously in diagnosis of DENV infection. Thus, with a panel of (eg) DENV-1 antisera, obtained from collaborators in Belem, Brazil or Panama City, Panama (who have already agreed to collaborate) or elsewhere, as well as control antisera or antisera from patients infected with agents other than DENV, we will be uniquely situated to sort through these thousands of epitopes while selecting for future diagnostic usage those that are frequently recognized by patients with DENV-1 but rarely if ever recognized by control antisera, or even by patients who were infected by DEN-2, 3, or 4. Inasmuch as current DENV serodiagnostics are of modest performance and do not make claims to be able to distinguish infections among the four DENV serotypes, this method offers the potential for truly disruptive improvements in DENV diagnosis. This aim is greatly aided by the very large numbers of DENV-specific epitopes that will be identified for each serotype in this method.

2. By systematically comparing the reactivity of the sera from patients who have experienced a secondary DENV infection (eg, DENV-2 infection some years after DENV-1 infection) to those who have had only a primary infection with DENV-1 or -2, we will identify those epitopes that are commonly or disproportionately recognized in such secondary infections, offering the possibility of specific diagnosis of secondary infections. No such capability exists presently.

3. By identifying epitopes in (2) above, the present invention offers the potential for identifying epitopes involved in ADE and thus intervening in ADE. The ability of these epitopes as well as those in primary infection to neutralize DENV can be characterized by raising antibodies against the corresponding VLP in animal models such as mice (Mus musculus). Our prior art has show such immunization to be able to routinely elicit high-titer antibodies. The mouse antisera can be studied for their ability to facilitate ADE.

4. DENV vaccines are not yet available, in large part because of the considerable challenge in evoking protective antibody responses against all 4 serotypes while having to assure, because of the potential that a vaccine will suffer from severe illness due to ADE caused by infection with a DENV type against which the vaccine did not protect, that strong and durable responses against all 4 serotypes will be virtually universally attained. By identifying thousands of protective epitopes, as determined by mouse-based or “in vitro immune system” based approaches for testing for neutralization, we will potentially expand manifold the number of candidate epitopes for vaccines, while offering the ready and known ability to evoke potent immunity against them (with RNA phage VLPs as immunogen) in mouse models while assessing their individual neutralizing capacities of the mouse antibodies. Those VLPs that evoke potent neutralizing responses against each DENV type can be identified, pooled and arrayed into a vaccine that is multi-component against all 4 DENV types. Such format significantly increases the likelihood that potent neutralizing responses can be evoked reliably against all four types while deploying the immunologically highly potent vehicle of a VLP that is inherently structurally optimal for evoking potent and specific anti-peptide responses.

### B. Random Peptide Library

1. The random-peptide library is composed of peptides of random sequence that are not informed in any way by the sequence of the proteome of the pathogen of interest. Libraries of 10¹⁰ complexity are routinely possible using (eg) MS2 VLP technology at modest scale. This library offers additional means of identifying neutralizing epitopes for vaccines as well as diagnostically useful epitopes, even those epitopes that are generated through the interaction of residues (aa or carbohydrate) that are non-contiguous on the pathogen genome (“folded” epitopes or carbohydrate/glycoprotein epitopes). Often such folded epitopes prove to be important neutralization determinants, and the random peptide library offers the potential to identify mimetics of epitopes that are not identifiable with other technologies, much less on a large-scale and systematic level.

2. Thus the present invention claims the ability to screen human serum antibodies against a pathogen and identify mimetics of linear or folded epitopes that are of diagnostic and/or vaccine importance, and to systematically screen such putative determinants in a high throughput, highly immunogenic context that will potentially identify a spectrum of neutralization determinants that in many cases will be unidentifiable or not systemically identifiable by other technologies.

Further Development of the VLP Technology.

The invention could enable the high-throughput screening of large numbers of candidate vaccine epitopes. Our methods for efficient synthesis of high complexity MS2 VLP libraries, coupled with recent developments in mega-scale oligonucleotide synthesis, and use of affinity selection and deep sequence analysis for epitope identification make it possible to generate a thorough catalog of epitopes for peptidomes much larger than that of dengue virus. Of course, we can already tackle genomes several times larger than dengue's by the simple expedient of designing libraries whose peptides scan the proteome in increments larger than the single amino acid steps we proposed above. Taking the next step beyond mere epitope identification—assigning a neutralization activity to each epitope—will require implementation of high-throughput immunization systems and neutralization assays. For dengue virus, as for many other agents, the necessary neutralization assays either exist already or can be readily devised. The current requirement for immunization of animals remains a significant bottleneck, but even this seems to be succumbing to new technology as in vitro immune systems begin to appear (note for example the technology being commercialized by the company called Vaxdesign). Even assuming the necessity of continued use of animals, it should be possible to apply this approach to pathogens with much larger genomes.

### Example 1

### References

1. Jonsson C B, Figueiredo L T, Vapalahti O: A global perspective on hantavirus ecology, epidemiology, and disease. Clin Microbiol Rev, 23:412-441.
2. Sui J, Hwang W C, Perez S, Wei G, Aird D, Chen L M, Santelli E, Stec B, Cadwell G, All M, et al: Structural and functional bases for broad-spectrum neutralization of avian and human influenza A viruses. Nat Struct Mol Biol 2009, 16:265-273.
3. Wang T T, Palese P: Universal epitopes of influenza virus hemagglutinins? Nat Struct Mol Biol 2009, 16:233-234.
4. Clapham P R, Lu S: Vaccinology: precisely tuned antibodies nab HIV. Nature, 477:416-417.
5. Wu X, Zhou T, Zhu J, Zhang B, Georgiev I, Wang C, Chen X, Longo N S, Louder M, McKee K, et al: Focused evolution of HIV-1 neutralizing antibodies revealed by structures and deep sequencing. Science, 333:1593-1602.
6. Wu X, Yang Z Y, Li Y, Hogerkorp C M, Schief W R, Seaman M S, Zhou T, Schmidt S D, Wu L, Xu L, et al: Rational design of envelope identifies broadly neutralizing human monoclonal antibodies to HIV-1. Science, 329:856-861.
7. Zhou T, Georgiev I, Wu X, Yang Z Y, Dai K, Finzi A, Kwon Y D, Scheid J F, Shi W, Xu L, et al: Structural basis for broad and potent neutralization of HIV-1 by antibody VRC01. Science, 329:811-817.
8. Walker L M, Phogat S K, Chan-Hui P Y, Wagner D, Phung P, Goss J L, Wrin T, Simek M D, Fling S, Mitcham J L, et al: Broad and potent neutralizing antibodies from an African donor reveal a new HIV-1 vaccine target. Science 2009, 326:285-289.
9. Walker L M, Huber M, Doores K J, Falkowska E, Pejchal R, Julien J P, Wang S K, Ramos A, Chan-Hui P Y, Moyle M, et al: Broad neutralization coverage of HIV by multiple highly potent antibodies. Nature, 477:466-470.
10. Scheid J F, Mouquet H, Ueberheide B, Diskin R, Klein F, Oliveira T Y, Pietzsch J, Fenyo D, Abadir A, Velinzon K, et al: Sequence and structural convergence of broad and potent HIV antibodies that mimic CD4 binding. Science, 333:1633-1637.
11. Lober C, Anheier B, Lindow S, Klenk H D, Feldmann H: The Hantaan virus glycoprotein precursor is cleaved at the conserved pentapeptide WAASA. Virology 2001, 289:224-229.
12. Wang Y, Boudreaux D M, Estrada D F, Egan C W, St Jeor S C, De Guzman R N: NMR structure of the N-terminal coiled coil domain of the Andes hantavirus nucleocapsid protein. J Biol Chem 2008, 283:28297-28304.
13. Arikawa J, Schmaljohn A L, Dalrymple J M, Schmaljohn C S: Characterization of Hantaan virus envelope glycoprotein antigenic determinants defined by monoclonal antibodies. J Gen Virol 1989, 70 (Pt 3):615-624.
14. Wang M, Pennock D G, Spik K W, Schmaljohn C S: Epitope mapping studies with neutralizing and non-neutralizing monoclonal antibodies to the G1 and G2 envelope glycoproteins of Hantaan virus. Virology 1993, 197:757-766.
15. Lundkvist A, Niklasson B: Bank vole monoclonal antibodies against Puumala virus envelope glycoproteins: identification of epitopes involved in neutralization. Arch Virol 1992, 126:93-105.
16. Lundkvist A, Horling J, Athlin L, Rosen A, Niklasson B: Neutralizing human monoclonal antibodies against Puumala virus, causative agent of nephropathia epidemica: a novel method using antigen-coated magnetic beads for specific B cell isolation. J Gen Virol 1993, 74 (Pt 7):1303-1310.
17. Botten J, Mirowsky K, Kusewitt D, Bharadwaj M, Yee J, Ricci R, Feddersen R M, Hjelle B: Experimental infection model for Sin Nombre hantavirus in the deer mouse (Peromyscus maniculatus). Proc Nall Acad Sci USA 2000, 97:10578-10583.
18. Bharadwaj M, Nofchissey R, Goade D, Koster F, Hjelle B: Humoral immune responses in the hantavirus cardiopulmonary syndrome. J Infect Dis 2000, 182:43-48.
19. Jenison S, Yamada T, Morris C, Anderson B, Torrez-Martinez N, Keller N, Hjelle B: Characterization of human antibody responses to four corners hantavirus infections among patients with hantavirus pulmonary syndrome. J Virol 1994, 68:3000-3006.
20. Yamada T, Hjelle B, Lanzi R, Morris C, Anderson B, Jenison S: Antibody responses to Four Corners hantavirus infections in the deer mouse (Peromyscus maniculatus): identification of an immunodominant region of the viral nucleocapsid protein. J Virol 1995, 69:1939-1943.
21. Khurana S, Suguitan A L, Jr., Rivera Y, Simmons C P, Lanzavecchia A, Sallusto F, Manischewitz J, King L R, Subbarao K, Golding H: Antigenic fingerprinting of H5N1 avian influenza using convalescent sera and monoclonal antibodies reveals potential vaccine and diagnostic targets. PLoS Med 2009, 6:e1000049.
22. Larman H B, Zhao Z, Laserson U, Li M Z, Ciccia A, Gakidis M A, Church G M, Kesari S, Leproust E M, Solimini N L, Elledge S J: Autoantigen discovery with a synthetic human peptidome. Nat Biotechnol, 29:535-541.
23. Caldeira J D, Medford A, Kines R C, Lino C A, Schiller J T, Chackerian B, Peabody D S: Immunogenic display of diverse peptides, including a broadly cross-type neutralizing human papillomavirus L2 epitope, on virus-like particles of the RNA bacteriophage PP7. Vaccine 2010, 28:4384-4393.
24. Peabody D S: Subunit fusion confers tolerance to peptide insertions in a virus coat protein. Arch Biochem Biophys 1997, 347:85-92.
25. Peabody D S, Manifold-Wheeler B, Medford A, Jordan S K, do Carmo Caldeira J, Chackerian B: Immunogenic display of diverse peptides on virus-like particles of RNA phage MS2. J Mol Biol 2008, 380:252-263.
26. Chackerian B, Caldeira Jdo C, Peabody J, Peabody D S: Peptide Epitope Identification by Affinity Selection on Bacteriophage MS2 Virus-Like Particles. J Mol Biol 2011, 409:225-237.
27. Kunkel T A: Rapid and efficient site-specific mutagenesis without phenotypic selection. Proc Natl Acad Sci USA 1985, 82:488-492.
28. Sidhu S S, Lowman H B, Cunningham B C, Wells J A: Phage display for selection of novel binding peptides. Methods Enzymol 2000, 328:333-363.
29. Bowden T A, Aricescu A R, Gilbert R J, Grimes J M, Jones E Y, Stuart D I: Structural basis of Nipah and Hendra virus attachment to their cell-surface receptor ephrin-B2. Nat Struct Mol Biol 2008, 15:567-572.
30. Xu K, Rajashankar K R, Chan Y P, Himanen J P, Broder C C, Nikolov D B: Host cell recognition by the henipaviruses: crystal structures of the Nipah G attachment glycoprotein and its complex with ephrin-B3. Proc Natl Acad Sci USA 2008, 105:9953-9958.
31. Gromowski G D, Barrett N D, Barrett A D: Characterization of dengue virus complex-specific neutralizing epitopes on envelope protein domain III of dengue 2 virus. J Virol 2008, 82:8828-8837.
32. Baker M: Microarrays, megasynthesis. Nature Methods 2011, 8:457-460.
33. Algire M, Krishnakumar R, Merryman C: Megabases for kilodollars. Nat Biotechnol, 28:1272-1273.
34. Kosuri S, Eroshenko N, Leproust E M, Super M, Way J, Li J B, Church G M: Scalable gene synthesis by selective amplification of DNA pools from high-fidelity microchips. Nat Biotechnol, 28:1295-1299.
35. Matzas M, Stahler P F, Kefer N, Siebelt N, Boisguerin V, Leonard J T, Keller A, Stahler C F, Haberle P, Gharizadeh B, et al: High-fidelity gene synthesis by retrieval of sequence-verified DNA identified using high-throughput pyrosequencing. Nat Biotechnol, 28:1291-1294.
36. Peabody D S: Translational repression by bacteriophage MS2 coat protein expressed from a plasmid. A system for genetic analysis of a protein-RNA interaction. J Biol Chem 1990, 265:5684-5689.
37. Peabody D S: The RNA binding site of bacteriophage MS2 coat protein. Embo J 1993, 12:595-600.
38. Binder M, Otto F, Mertelsmann R, Veelken H, Trepel M: The epitope recognized by rituximab. Blood 2006, 108:1975-1978.
39. Sabo J K, Keizer D W, Feng Z P, Casey J L, Parisi K, Coley A M, Foley M, Norton R S: Mimotopes of apical membrane antigen 1: Structures of phage-derived peptides recognized by the inhibitory monoclonal antibody 4G2dc1 and design of a more active analogue. Infect Immun 2007, 75:61-73.
40. Irving M B, Craig L, Menendez A, Gangadhar B P, Montero M, van Houten N E, Scott J K: Exploring peptide mimics for the production of antibodies against discontinuous protein epitopes. Mol Immunol 2010, 47:1137-1148.
41. Botten J, Mirowsky K, Kusewitt D, Ye C, Gottlieb K, Prescott J, Hjelle B: Persistent Sin Nombre virus infection in the deer mouse (Peromyscus maniculatus) model: sites of replication and strand-specific expression. J Virol 2003, 77:1540-1550.
42. Hughes J B, Hellmann J J, Ricketts T H, Bohannan B J: Counting the uncountable: statistical approaches to estimating microbial diversity. Appl Environ Microbiol 2001, 67:4399-4406.
43. Srivastava S, Chen L: A two-parameter generalized Poisson model to improve the analysis of RNA-seq data. Nucleic Acids Res, 38:e170.
44. Tumban E, Peabody J, Peabody D S, Chackerian B: A pan-HPV vaccine based on bacteriophage PP7 VLPs displaying broadly cross-neutralizing epitopes from the HPV minor capsid protein, L2. PLoS One, 6:e23310.
45. Bharadwaj M, Lyons C R, Wortman I A, Hjelle B: Intramuscular inoculation of Sin Nombre hantavirus cDNAs induces cellular and humoral immune responses in BALB/c mice. Vaccine 1999, 17:2836-2843.
46. Ray N, Whidby J, Stewart S, Hooper J W, Bertolotti-Ciarlet A: Study of Andes virus entry and neutralization using a pseudovirion system. J Virol Methods, 163:416-423.

### Plasmid Examples

### Experimental Overview for Examples 2 and 3

Two plasmid vectors that facilitate the construction of random-sequence peptide libraries on virus-like particles (VLP's) of bacteriophage MS2 are described. The first, pDSP1, was constructed for convenient cloning of PCR-generated—or other double-stranded DNA—fragments into the AB-loop of the downstream copy of a coat protein single chain dimer. The second is called pDSP62 and was constructed specifically for introduction of peptide sequences at virtually any position in the single-chain dimer (usually the AB-loop) by the site-directed mutagenesis method of Kunkel et al. (1). The general features of the plasmids are presented below.

### Example 2

pDSP1—a Plasmid Expressing a Single-Chain Dimer with Convenient Cloning Sites for Insertion in the AB-Loop

The plasmid pDSP1 (see FIG. 1X) contains the T7 transcription signals of pET3d and the kanamycin resistance and replication origin of pET9d. [Information regarding pET3d and pET9d may be found at the New England Biolabs vector database, https://www.lablife.org/ct?f=v&a=listvecinfo). It expresses the coding sequence of the MS2 single-chain coat protein dimer (2), modified to contain unique SalI and KpnI restriction sites. This facilitates simple cloning of foreign sequences into the AB-loop. To make these sites unique, it was necessary to destroy other SalI and KpnI sites in the vector and in the upstream coat sequence.

The MS2 coat sequence in the vicinity of the AB-loop insertion site for pDSP1 is shown below. Note the presence of SalI and KpnI sites.

. . . 6 7 8 9 10 11 12 13 14 15 16 17 18 19 20 21 22 . . .
. . . GlnPheValLeuValAspAsnGlyGlyThrGlyAspValThrValAlaPro . . .
. . . CAGTTCGTTCTCGTCGACAATGGCGGTACCGGCGACGTGACTGTCGCCCA . . .
SalI KpnI

Shown below is an example of an unspecified 7-mer insertion in pDSP1, with the random sequence inserted in the so-called 13/16 mode (i.e. between residues 13 and 16 of MS2 coat protein) using the PCR-based method of FIG. 1X.

. . . 6 7 8 9 10 11 12 13 16 17 18 19 20 21 22 . . .
. . . GlnPheValLeuValAspAsnGly X X X X X X X GlyAspValThrValAlaPro . . .
. . . CAGTTCGTTCTCGTCGACAATGGCnnnnnnnnnnnnnnnnnnnnnGGCGACGTGACTGTCGCCCCA . . .
SalI

In this PCR-based method the 5′-PCR primer shown below would be used with the 3′ primer 12013 to generate a fragment of the coat protein coding sequence with the random sequence inserted between amino acids 13 and 16. N=A, C, G, or T and S=G or C. After digestion with SalI and BamHI, the fragment would be inserted between SalI and BamHI of pDSP1. Shown below is an example of a 5′-primer that could be used to generate such a library:

5′-CGCGTCGACAATGGC(NNS)₇GGCGACGTGACTGTCGCCCCA-3′

With pDSP1, random-sequence peptide libraries are usually constructed by cloning into the AB-loop a PCR fragment generated using a monomeric coat protein sequence as template (e.g. pMCT). A synthetic oligonucleotide 5′-primer is designed to attach a SalI (or KpnI) site and a sequence of codons specified by the synthetic primer DNA (e.g. 6-10 codons taken from the sequence of an antigen or potential antigen) to a site just upstream of the AB-loop. A 3′-primer anneals to sequences in the plasmid vector just downstream of BamHI. The resulting PCR product is digested with SalI (or KpnI) and BamHI and cloned at the corresponding sites of pDSP1. This results in insertion of peptides into the AB-loop, the exact site of insertion depending on the specific design of the 5′-primer. For most insertions use of the SalI site is preferred as it affords more flexibility that KpnI in selection of the insertion site. With these methods it is relatively straightforward to produce peptide VLP libraries with up to 10⁸-10⁹ individual members.

### Example 3

### pDSP62—a Plasmid Suitable for Library Construction Using Efficient Site-Directed Mutagenesis Methods

pDSP62 Differs from pDSP1 Primarily by the Introduction of an M13 Origin of Replication.

Methods for library production like that described above for pDSP1, are difficult to scale up, because it is inconvenient to purify DNA restriction fragments in the necessary quantities. Moreover, during ligation reactions some of the DNA is inevitably diverted into useless side-products, reducing the yield of the desired plasmid. The construction of complex libraries would be facilitated by methods that efficiently produce larger yields of the correct recombinant DNA than are found in a typical ligation reaction. Specifically, we want to make use of a variation of an old method for site-directed mutagenesis, which has been used already by others to produce peptide libraries on filamentous phage in the 10¹¹ complexity range (1,3). The method is applied to single-stranded circular DNAs produced from a particular kind of plasmid (also know as a phagemid) that contains an M13 origin of replication. Infection with an M13 helper phage (e.g. M13K07) of a dut⁻, ung⁻ strain (e.g. BW313) containing the plasmid results in facile production of dUTP-substituted single-stranded DNA. In the actual mutagenesis reaction, a mismatched oligonucleotide primer is annealed to the single-stranded DNA template and is elongated using a DNA polymerase (e.g. that of T7 phage). The DNA is ligated to produce closed circular DNA, and introduced by transformation into and ung⁺ strain, where the mutant strand is preferentially replicated. My experience in the production of peptide-VLP libraries indicates that typically about 90% of the transformants contain the desired peptide insertions. The primer extension mutagenesis reaction can be conducted on relatively large quantities of DNA (e.g. 20 ug), enough to readily generate on the order of 10¹¹ individual recombinants by electroporation. Such high complexities are important in the construction of libraries of totally random sequences, but are unlikely to be necessary for production of most antigen fragment libraries. Nonetheless this method can also be more convenient that the PCR-based technique and so is also described here.

To facilitate the production of single-stranded DNA, we introduced an M13 origin of replication into pDSP1. To do so, the M13 origin found in pUC119 was amplified by PCR and cloned at a unique AlwNI site in pDSP1. This plasmid, called pDSP1-IG, is the progenitor to pDSP62. Because it is only an intermediate to the construction of pDSP62 I, its sequence is not shown.

Targeting Insertions to Only One Half of the Single-Chain Dimer Through the Use of a Synthetic “Codon-Juggled” Coat Gene.

The desire to use primer-extension mutagenesis for efficient peptide library construction introduced a new complication. Our display method relies on the ability to specifically introduce foreign peptides into only one of the two AB-loops of the single-chain dimer. Using the single-chain dimer sequence present in pDSP1, the mutagenic primer would anneal to sequences in both halves, resulting in double insertions. But we already know that insertions in both AB-loops result in a high frequency of protein folding failures. Moreover, even if the insertions were tolerated, an site-directed mutagenesis that failed to target only one half of the single-chain dimer would result in the display of two different peptides on each VLPs. For these reasons, a “codon-juggled” version of coat protein was synthesized and exchanged it for the normal upstream half of the single-chain dimer. The codon-juggled sequence contains the maximum possible number of silent nucleotide substitutions, and thus produces a polypeptide having the wild-type coat protein amino acid sequence. However, the presence of numerous mutations makes the juggled sequence incapable of efficiently annealing to the mutagenic oligonucleotide, and therefore the mutagenic primer is specifically directed to the downstream AB-loop sequence. Plasmid pDSP62 is shown in FIG. 2X and its sequence is provided in FIG. 3X.

A Chloramphenicol-Resistant M13 Helper Phage for Single-Strand pDSP62 Production.

Plasmid pDSP62 confers resistance to kanamycin. The helper phages (e.g. M13KO7) usually used for production of single stranded phagemid DNA also confer kanamycin resistance, and are therefore unsuitable for use with the plasmids described here. For this reason we constructed M13CM1, a chloramphenicol resistant derivative of M13KO7. The chloramphenicol resistance gene of pACYC184(4) (4) was amplified by PCR using primers that attached recognition sequences for XhoI and Sad, and the fragment was inserted into M13KO7 in place of its kanamycin resistance gene, taking advantage of XhoI and Sad sites that roughly flank the kanamycin resistance determinant. In the presence of kanamycin (selects for pDSP62 maintenance) and chloramphenicol (selects for helper phage), cells produce large quantities of single-stranded plasmid DNA after infection with M13 CM1. Using these single-stranded templates and the method of Kunkel et al. (1), random sequence peptide libraries have been readily produced that contain more than 10¹⁰ individual members for [NNS]₆, [NNS]₇, [NNS]₈ and [NNS]₁₀. Significantly higher complexities are possible with scale-up.

### References for Examples 2 and 3

1. Kunkel, T. A., Bebenek, K., and Mcclary, J. (1991) Methods in Enzymology 204, 125-139
2. Peabody, D. S., and Lim, F. (1996) Nucleic Acids Res 24, 2352-2359
3. Sidhu, S. S., Lowman, H. B., Cunningham, B. C., and Wells, J. A. (2000) Methods Enzymol 328, 333-363
4. Chang, A. C., and Cohen, S. N. (1978) J Bacteriol 134, 1141-1156

### Example 4

### Design of a PP7 Peptide Display Vector

Two general kinds of plasmid were constructed for the synthesis of PP7 coat protein in E coli (see FIGS. 1X and 5X). The first expresses coat protein from the lac promoter and is used (in combination with pRZP7—see below) to assay for coat protein's tolerance of peptide insertions using translational repressor and VLP assembly assays. The second plasmid type expresses the protein from the T7 promoter and transcription terminator. These plasmids produce large amounts of coat protein that assembles correctly into a VLP. They also produce coat-specific mRNA with discrete 5′- and 3′-termini for encapsidation into VLPs.

Design of the Peptide Insertion Site.

The three-dimensional structure of the PP7 capsid shows that it is comprised of a coat protein whose tertiary structure closely mimics that of MS2, even though the amino acid sequences of the two proteins show only about 12% sequence identity (4). The PP7 protein possesses an AB-loop into which peptides may be inserted following a scheme similar to the one we described previously for MS2 (1). As in the MS2 case, we began by mutating the PP7 coat sequence to contain a site for the restriction endonuclease KpnI, thus facilitating insertion of foreign sequences in the plasmid we call pP7K (FIG. 1X). This modification resulted in the amino acid substitution (E 11T) shown in FIG. 2X. This substitution was well tolerated, since the mutant coat protein represses translation and assembles correctly into a VLP. Again following the MS2 example, we assumed that the folding of a single chain dimer version of PP7 coat protein would be more resistant to AB-loop insertions than the conventional dimer. Its construction was described previously (2), but here we describe its use for peptide display for the first time. We modified the single-chain dimer to contain a KpnI site only in the downstream copy of the coding sequence, producing p2P7K32 (FIG. 1X). In this design we inserted peptides at amino acid 11, but it should be noted that other specific insertion sites are possible, possibly anywhere within the AB-loop. In fact, below we describe tests of two alternative insertion modes (FIGS. 2X and 3X). The first we call the 11/11 mode because the 11^{th} amino acid appears twice—as thr on the N-terminal side of the inserted peptide, and as the wild-type glu11 on the C-terminal side. In the so-called 11/12 mode the insertion is flanked by thr11 on one side and ala12 on the other.

To test the general tolerance of PP7 coat protein to AB-loop insertions, we created libraries of random sequence peptides inserted in the AB-loop of PP7 coat protein using the scheme shown in FIG. 3X. The random sequences consisted of 6, 8 or 10 copies of the sequence NNY (where N is any nucleotide and Y is pyrimidine). Such libraries contain 15 of the possible amino acids, and are therefore capable of substantial diversity. However, by avoiding the possibility of stop codons subsequent analysis is greatly facilitated.

A PP7 coat protein-producing analog of pDSP62 has been produced. Called pDSP7, it contains the same functional features as pDSP62, but replaces the MS2 coat protein-encoding sequences with a PP7 single-chain dimer sequence in which the upstream half has been codon-juggled and the downstream half is essentially the wild-type sequence. Because it produced single-stranded DNA in cells infected with M13CM1, it is amenable to the site-directed mutagenesis method for library construction described above. A simple variant called pDSP7K inserts a KpnI site at the AB loop, thus facilitating the application of the PCR-based library construction method, also described above.

### Peptides Displayed on MS2 and PP7 VLPs are Displayed to the Immune System and are Immunogenic.

We constructed PP7 VLPs displaying the specific peptide sequences shown in FIGS. 2X and 7X. These included the so-called Flag peptide and sequences derived from the minor capsid protein L2 of human Papillomavirus type 16 (HPV16), the V3 loop of HIV-1 gp120, and Bacillus anthracis protective antigen. To demonstrate that these inserted peptides were indeed displayed on the surface of VLPs, we assessed the ability of a monoclonal antibody against HPV16 L2 (called RG-1) to bind to PP7 L2-VLPs by ELISA. As shown in FIG. 11X, RG-1 bound to L2-VLPs, but not to PP7 VLPs that displayed the V3 peptide. To demonstrate the immunogenicity of the VLPs, mice were immunized with V3-VLPs by intramuscular injection as described previously (1). As shown in FIG. 12X, sera from the mice were tested by ELISA and shown to have high titer IgG antibodies that specifically react with a synthetic version of the V3 peptide. Similar experiments conducted using the HIV V3 and macaque CCR5 ECL2 sequences gave similar results as described in Peabody, D. S., Manifold-Wheeler, B., Medford, A., Jordan, S. K., do Carmo Caldeira, J., and Chackerian, B. (2008) J Mol Biol 380, 252-263

### References for Example 4

1. Peabody, D. S., Manifold-Wheeler, B., Medford, A., Jordan, S. K., do Carmo Caldeira, J., and Chackerian, B. (2008) J Mol Biol 380, 252-263
2. Caldeira, J. C., and Peabody, D. S. (2007) J Nanobiotechnology 5, 10
3. Olsthoorn, R. C., Garde, G., Dayhuff, T., Atkins, J. F., and Van Duin, J. (1995) Virology 206, 611-625
4. Tars K, F. K., Bundule M, Liljas L. (2000) Virology 272, 331-337
5. Lim, F., Downey, T. D., and Peabody, D. S. (2001) Journal of Biological Chemistry 276, 22507-22512
6. Peabody, D. S. (1990) J Biol Chem 265, 5684-5689

1. A method of screening immunogenic viral like particles comprising:
(a) providing a library of peptides which have been expressed on virus-like particles comprising a bacteriophage single chain coat polypeptide dimer, preferably a bacteriophage MS2 single chain coat polypeptide dimer or a bacteriophage PP7 single coat polypeptide dimer, each of said peptides corresponding to a putative epitope of a pathogen, said virus-like particles having been made by prokaryotically expressing a plurality of nucleic acid constructs which each comprise an oligonucleotide encoding one of the peptides corresponding to the putative pathogenic epitope;
(b) conducting affinity selection on the library of virus-like particles using monoclonal or polyclonal antibodies (of any class or subclass including but not restricted to IgG, IgM, IgY or IgE of any vertebrate species) or antiserum to select candidate virus-like particles wherein said antiserum is reactive to epitopes on a pathogen, is reactive to epitopes which produce autoantibody responses, or is reactive to epitopes which produce responses to vaccines or other foreign antigens, including allergens; and
(c) sequencing the peptides which have been expressed on the candidate virus-like particles; and further optionally
(d) prokaryotically expressing the sequenced peptides on virus-like particles comprising a bacteriophage MS2 single chain coat polypeptide dimer or a bacteriophage PP7 single chain coat polypeptide dimer and thereafter purifying the virus-like particles comprising the sequenced peptides; and further optionally
(e) immunizing a subject with the purified virus-like particles comprising the sequenced peptides and assaying the subject's immune response upon exposure to the pathogen.

2. The method of claim 1, wherein the pathogen is a virus, a bacterium, a parasite, or a microbe.

3. The method of claim 1, wherein the pathogen is selected from the group consisting of dengue virus; yellow fever virus; West Nile virus; Japanese encephalitis virus; HIV; HTLV-I, Bunyaviridae viruses including the hantaviruses, Crimean-Congo hemorrhagic fever, Rift Valley fever virus, and fever and severe fever and thrombocytopenia virus; arenaviruses including all agents of South American hemorrhagic fever, Lassa virus and lymphocytic choriomeningitis virus; filoviruses including Ebola and Marburg viruses; paramyxoviruses including morbilliviruses, henipaviruses, respiroviruses including RSV and metapneumovirus and rubellaviruses; Alphaviruses including Chikungunya, O'nyung-nyung, Semliki Forest, Ross River, Sindbis, eastern, western and Venezuelan equine encephalitis; picornaviruses; papillomaviruses including HPV; herpesviruses including HSV-1/2, EBV, CMV, HHV-6, 7, and 8; polyomaviruses including SV40, JC and BK viruses; poxviruses including variola and vaccinia viruses; bacterial pathogens including any human pathogen such as Staphylococcus spp; Streptococcus spp; E. coli and other pathogenic coliforms; and parasitic pathogens include malaria (Plasmodium spp).

4. The method of claim 1 wherein said autoantibody response occurs in systemic lupus erythematosus, rheumatoid arthritis (RA), juvenile RA, Hashimoto throiditis, Addison's disease, “antiphospholipid syndrome”, autoimmune hepatitis, autoimmune thrombocytopenia, bullous pemphigoid, dermatomyositis, Goodpasteur's disease, Lambert-Eaton myasthenia, multiple sclerosis, myasthenia gravis, pemphigus vulgaris, polymyositis, primary biliary cirrhosis, psoriasis, Sjögren's disease or type-1 diabetes.

5. The method of claim 1 wherein said foreign antigens are allergens.

6. The method according to claim 5 wherein said allergen is DCP-1, or another known domestic animal, plant, fungal, or arthropod allergen,

7. The method according to claim 1 wherein said bacteriophage comprises MS2 single chain coat polypeptide dimer or PP7 single chain coat polypeptide dimer.

8. The method of claim 7, wherein the library of peptides which have been expressed on virus-like particles comprising a bacteriophage MS2 single chain coat polypeptide dimer or a bacteriophage PP7 single chain coat polypeptide dimer is either an antigen fragment library or a random sequence library.

9. The method of claim 1, wherein peptides which have been expressed on selected virus-like particles are sequenced using deep sequencing.

10. The method of claim 1, wherein:
(a) the library of peptides which have been expressed on virus-like particles comprising a bacteriophage MS2 single chain coat polypeptide dimer or a bacteriophage PP7 single chain coat polypeptide dimer is a random sequence library; and
(b) the pathogen is a virion containing a glycoprotein that maps to a sequenced peptide of a purified virus-like particle.

11. The method of claim 10, wherein the virion is the Dengue virion.

12. The method of claim 1, wherein:
(a) the library of peptides which have been expressed on virus-like particles comprising a bacteriophage MS2 single chain coat polypeptide dimer or a bacteriophage PP7 single chain coat polypeptide dimer is a random sequence library; and
(b) the pathogen contains a carbohydrate that maps to a sequenced peptide of a purified virus-like particle.

13. The method of claim 12, wherein the pathogen is malaria protein AMA-1.

14. The method of claim 1, wherein assaying the subject's immune response upon exposure to the pathogen is conducted in vitro.

15. The method of claim 14, wherein assaying the subject's immune response upon exposure to the pathogen is conducted using a neutralization assay.

16. The method of claim 1, wherein:
(a) the library of peptides which have been expressed on virus-like particles comprising a bacteriophage MS2 single chain coat polypeptide dimer or a bacteriophage PP7 single chain coat polypeptide dimer is an antigen fragment library; and
(b) peptides which have been expressed on virus-like particles in the antigen fragment library have been prokaryotically expressed using a plurality of oligonucleotides which in the aggregate scan the pathogen's genome.

17. The method of claim 16, wherein the pathogen is influenza (any subtype) or hantavirus, including SNV.

18. The method of claim 16, wherein each oligonucleotide is approximately 15-30 mer in length.

19. The method of claim 15, wherein the neutralization assay is a pseudotype neutralization assay.

20. The method of claim 1, wherein the method is conducted in a high-throughput format.

21. The method of claim 1, wherein the step of immunizing a subject with the purified virus-like particles comprising the sequenced peptides is conducted in vivo, in vitro or in silica.

22. The method of claim 1, wherein affinity selection on the library of virus-like particles using pathogen antiserum comprises comparing an affinity range for a variety of neutralizing antibody titers.

23. The method of claim 1, wherein the pathogen has more than one serotype and the peptides corresponding to a putative epitope of the pathogen include peptides corresponding to epitopes of each pathogen serotype.

24. The method of claim 1, wherein the pathogen antiserum is a polyclonal antiserum.

25. The method of claim 1, wherein:
(a) affinity selection on the library of virus-like particles uses pathogen antiserum which comprises comparing an affinity range for a variety of neutralizing antibody titers;
(b) the pathogen has more than one serotype and the peptides corresponding to a putative epitope of the pathogen include peptides corresponding to epitopes of each pathogen serotype; and
(c) the pathogen antiserum is a polyclonal antiserum.

26. The method of claim 25, wherein the pathogen is Dengue viruses DENV-1 through 4.

27. The method of claim 26, wherein the peptides have been expressed on virus-like particles comprising a bacteriophage MS2 single chain coat polypeptide dimer.

28. The method of claim 12, wherein the pathogen is a microbe.

29. The method of claim 1, wherein the step of prokaryotically expressing the sequenced peptides on virus-like particles, wherein said particles comprise a bacteriophage MS2 single chain coat polypeptide dimer or a bacteriophage PP7 single chain coat polypeptide dimer, comprises:
(a) reverse-translating the peptides which have been expressed on the candidate virus-like particles to ascertain one or more nucleic acid sequences which encode those peptides; and
(b) constructing a library of virus-like particles by (1) providing a plurality of nucleic acid constructs, treating the nucleic acid constructs with a restriction enzyme, and inserting the one or more nucleic acid sequences ascertained in step (a) into the nucleic acid constructs to obtain a population of transcription units (2) generating virus-like particles by expressing the transcription units in a prokaryote which has been modified to under-express an affinity tag, and
(3) purifying the viral-like particles using the affinity tag and isolating the library.

30. The method of claim 1, wherein in step (a), the peptides comprise a series of peptide mer units comprising between about 5 to about 20 amino acids, and wherein the series overlaps peptide regions adjacent to the putative epitope of the peptide sequences by between about 2 to about 10 amino acids.

31. The method of claim 1, wherein the nucleic acid construct comprises
(a) a bacterial or bacteriophage promoter which is operably associated with a coding sequence of either bacteriophage MS2 single chain coat polypeptide dimer or bacteriophage PP7 single chain coat polypeptide dimer, wherein the coat polypeptide dimer coding sequence (1) is modified to define a first restriction site positioned 5′ to that portion of the sequence which defines the coat polypeptide dimer AB loop, and (2) comprises (i) an oligonucleotide encoding one of the peptides, and (ii) a stop codon which (I) substitutes for that codon which would otherwise encode the coat polypeptide's first amino acid, or (II) which is positioned at the C-terminus of the single-chain dimer;
(b) a restriction site positioned 3′ to the coat polypeptide dimer coding sequence;
(c) a PCR primer positioned 3′ to the second restriction site;
(d) a repressor to resistance to a first antibiotic, wherein the repressor is operably associated with the promoter;
(e) a helper phage gene modified to contain a gene conferring resistance to a second antibiotic, and
(f) a replication origin for replication in a prokaryotic cell.

32. The method of claim 31, wherein a first primer is positioned 5′ to the first restriction site and a second primer is positioned 3′ to the second restriction site and 5′ to the PCR primer.

33. The method of claim 32, wherein the bacterial or bacteriophage promoter is a T7 promoter, the first restriction site is either a SalI and KpnI restriction site, the second restriction site is a BamHI site, the PCR primer is TP7, the antibiotic repressor is a kanamycin resistance gene, and the replication origin is colE1 ori.

34. The method of claim 31, wherein the construct further comprises a transcription terminator positioned 5′ to the second restriction site.

35. The method of claim 31, wherein the construct optionally comprises a transcription terminator positioned 5′ to the second restriction site.

36. The method of claim 31, wherein the bacterial or bacteriophage promoter is a T7 promoter, the RNA bacteriophage single chain coat polypeptide dimer is a MS2 coat protein single chain dimer, the codon sequence contains the maximum possible number of silent nucleotide substitutions, the restriction site is a BamHI site, the PCR primer is TP7, the repressor to resistance to a first antibiotic is a kanamycin resistance gene, the helper phage gene is modified to contain a gene conferring resistance to chloramphenicol, and the replication origin is colE1 ori.

37. The method of claim 1, wherein peptides corresponding to said putative epitope in step (a) and sequenced peptides in step (e) are displayed on virus-like particles and encapsidate either MS2 mRNA or PP7 mRNA.

38. The method of claim 37 wherein said putative epitope is a pathogen putative epitope.

39. The method of claim 37 wherein said putative epitope is an allergen putative epitope.

40. The method of claim 37 wherein said putative epitope is an epitope which produces an autoantibody response or a response to a foreign antigen or an allogenic protein.

41. An immunogenic composition comprising a population of purified virus-like particles identified by the method of claim 1.

42. (canceled)

43. (canceled)

44. A method comprising characterizing an immune response of a sample, the method comprising contacting the sample with purified virus-like particles identified by the method of claim 1 and assaying any resultant immune response.

45. The method of claim 44, wherein the sample is obtained from a mammal that may be infected with a virus, a bacterium, a parasite, or a microbe.

46. The method of claim 44, wherein the mammal may be infected by H5N1, SNV, or Dengue viruses DENY-1 through 4.

47. (canceled)

48. A method of identifying a cryptic neutralizing epitope comprising:
(a) defining one or more potential viral cryptic neutralizing epitope-targeting peptide sequences;
(b) determining peptide sequences for each of the defined potential viral cryptic neutralizing epitope-targeting peptide sequences;
(c) make cDNA from and amplify the foreign insert from phage RNA to determine the RNA sequence and that of the peptide encoded by that insert;
(d) constructing a library of virus-like particles by (i) preparing nucleic acid constructs, treating the nucleic acid constructs with a restriction enzyme, and inserting the one or more nucleic acid sequences ascertained in step (c) into the nucleic acid constructs to obtain a population of transcription units (ii) generating virus-like particles by expressing the transcription units in a prokaryote which has been modified to under-express an affinity tag, and (iii) purifying the viral-like particles using the affinity tag and isolating the library;
(e) immunizing a subject with one or more of the viral-like particles; and
(f) identifying one or more viral-like particles that induce a neutralizing antibody response in the subject.

49. The method of claim 48, wherein more than one potential cryptic neutralizing epitope-targeting peptide sequence is defined and each of said sequences corresponds to overlapping peptides from throughout the targeted sequences.

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. The method of claim 48, wherein the cryptic neutralizing epitope-targeting peptide sequence targets a peptide selected from the group consisting of an HIV peptide, a self antigen, Flag peptide, amino acid sequences derived from the minor capsid protein L2 of human Papillomavirus type 16 (HPV16), the V3 loop of HIV-1 gp120, Bacillus anthracis protective antigen, a receptor, a ligand which binds to a cell surface receptor, a peptide with affinity for either end of a filamentous phage particle specific peptide, a metal binding peptide or a peptide with affinity for the surface of either MS2 or PP7.

62. The method of claim 61, wherein the method is conducted in a high-throughput format.

63. A method of making a vaccine comprising isolating viral-like particles that have been identified as inducing a neutralizing antibody response in the subject in accordance with the method of claim 48.

64. The method of claim 63, wherein the method is conducted in a high-throughput format.

65. A vaccine comprising viral-like particles that have been identified as inducing a neutralizing antibody response in the subject in accordance with the method of claim 48.

66. The method of claim 48, wherein the step of immunizing a subject is conducted in silica or in vitro.

67. (canceled)

68. (canceled)

69. (canceled)

70. The nucleic acid construct of claim 67, wherein the construct further comprises a transcription terminator positioned 5′ to the second restriction site.

71. The nucleic acid construct of claim 67, wherein the construct optionally comprises a transcription terminator positioned 5′ to the second restriction site.

72.-87. (canceled)


SEQUENCE LISTING (36 sequences)

SEQ ID NO: 1 - DNA, 5096 bp, Artificial Sequence
  synthetic sequence of pDSP1
ttcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactatagggagaccacaacggtttccctctagaaataattttgtttaactttaagaaggagatataccatggcttctaactttactcagttcgttctcgttgacaatggcggaactggcgacgtgactgtcgccccaagcaacttcgctaacggggtcgctgaatggatcagctctaactcgcgttcacaggcttacaaagtaacctgtagcgttcgtcagagctctgcgcagaatcgcaaatacaccatcaaagtcgaggtgcctaaagtggcaacccagactgttggtggtgtagagcttcctgtagccgcatggcgttcgtacttaaatatggaactaaccattccaattttcgctacgaattccgactgcgagcttattgttaaggcaatgcaaggtctcctaaaagatggaaacccgattccctcagcaatcgcagcaaactccggcctctacggcaactttactcagttcgttctcgtcgacaatggcggtaccggcgacgtgactgtcgccccaagcaacttcgctaacggggtcgctgaatggatcagctctaactcgcgttcacaggcttacaaagtaacctgtagcgttcgtcagagctctgcgcagaatcgcaaatacaccatcaaagtcgaggtgcctaaagtggcaacccagactgttggtggtgtagagcttcctgtagccgcatggcgttcgtacttaaatatggaactaaccattccaattttcgctacgaattccgactgcgagcttattgttaaggcaatgcaaggtctcctaaaagatggaaacccgattccctcagcaatcgcagcaaactccggcatctactaatagacgccgggttaattaattaaggatccggctgctaacaaagcccgaaaggaagctgagttggctgctgccaccgctgagcaataactagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgaa

SEQ ID NO: 2 - DNA, 4486 bp, Artificial Sequence
  synthetic sequence of pDSP62
ttcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcttttcaaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagtttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactatagggagaccacaacggtttccctctagaaataattttgtttaactttaagaaggagatataccatggcaagcaatttcacgcaatttgtattggtagataacgggggtacgggggatgttacggtagcaccttcaaattttgcaaatggtgtagcagagtggatatcaagcaatagcagaagccaagcatataaggttacgtgctcagtaagacaatcaagcgctcaaaacagaaagtatacgataaaggtagaagttccgaaggttgctacgcaaacggtaggtggtgttgaattgccggttgcagcttggagaagctatctcaacatggagttgacgatacctatatttgcaaccaacagtgattgtgaattgatagtaaaagctatgcaggggttgttgaaggacggtaatcctataccgagcgctatagctgctaatagtggcctctacggcaactttactcagttcgttctcgtcgacaatggcggaactggcgacgtgactgtcgccccaagcaacttcgctaacggggtcgctgaatggatcagctctaactcgcgttcacaggcttacaaagtaacctgtagcgttcgtcagagctctgcgcagaatcgcaaatacaccatcaaagtcgaggtgcctaaagtggcaacccagactgttggtggtgtagagcttcctgtagccgcatggcgttcgtacttaaatatggaactaaccattccaattttcgctacgaattccgactgcgagcttattgttaaggcaatgcaaggtctcctaaaagatggaaacccgattccctcagcaatcgcagcaaactccggcatctactaatagacgccgggttaattaattaggatccggctgctaacaaagcccgaaaggaagctgagttggctgctgccaccgctgagcaataactagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgaa

SEQ ID NO: 3 - DNA, 8599 bp, Artificial Sequence
  synthetic sequence of M13CM1
aacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaaagggtgagaaaggccggagacagtcaaatcaccatcaatatgatattcaaccgttctagctgataaattcatgccggagagggtagctatttttgagaggtctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaaacgttaatattttgttaaaattcgcgttaaatttttgttaaatcagctcattttttaaccaataggccgaaatcggcaaaatcccttataaatcaaaagaatagcccgagacctgcagggggggggggggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgagtgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattgagctcgaatcggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcgatccgtcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttggaatcagagcgggagctaaacaggaggccgattaaagggattttagacaggaacggtacgccagaatcttgagaagtgtttttataatcagtgaggccaccgagtaaaagagtctgtccatcacgcaaattaaccgttgtagcaatacttctttgattagtaataacatcacttgcctgagtagaagaactcaaactatcggccttgctggtaatatccagaacaatattaccgccagccattgcaacaggaaaaacgctcatggaaatacctacattttgacgctcaatcgtctgaaatggattatttacattggcagattcaccagtcacacgaccagtaataaaagggacattctggccaacagagatagaacccttctgacctgaaagcgtaagaatacgtggcacagacaatatttttgaatggctattagtctttaatgcgcgaactgatagccctaaaacatcgccattaaaaataccgaacgaaccaccagcagaagataaaacagaggtgaggcggtcagtattaacaccgcctgcaacagtgccacgctgagagccagcagcaaatgaaaaatctaaagcatcaccttgctgaacctcaaatatcaaaccctcaatcaatatctggtcagttggcaaatcaacagtagaaaggaattgaggaaggttatctaaaatatctttaggtgcactaacaactaatagattagagccgtcaatagataatacatttgaggatttagaagtattagactttacaaacaattcgacaactcgtattaaatcctttgcccgaacgttattaattttaaaagtttgagtaacattatcattttgcggaacaaagaaaccaccagaaggagcggaattatcatcatattcctgattatcagatgatggcaattcatcaatataatcctgattgtttggattatacttctgaattatggaaggaattgaaccaaccatatcaaaattattagcacgtaaaacagaaataaagaaattgcgtagattttcaggtttaacgtcagatgaatatacagtaacagtaccttttacatcgggagaaacaataacggattcgcctgattgctttgaataccaagttacaaaatcgcgcagaggcgaattattcatttcaattacctgagcaaaagaagatgatgaaacaaacatcaagaaaacaaaattaattacatttaacaatttcatttgaattaccttttttaatggaaacagtacataaatcaatatatgtgagtgaataaccttgcttctgtaaatcgtcgctattaattaattttcccttagaatccttgaaaacatagcgatagcttagattaagacgctgagaagagtcaatagtgaatttatcaaaatcataggtctgagagactacctttttaacctccggcttaggttgggttatataactatatgtaaatgctgatgcaaatccaatcgcaagacaaagaacgcgagaaaactttttcaaatatattttagttaatttcatcttctgacctaaatttaatggtttgaaataccgaccgtgtgataaataaggcgttaaataagaataaacaccggaatcataattactagaaaaagcctgtttagtatcatatgcgttatacaaattcttaccagtataaagccaacgctcaacagtagggcttaattgagaatcgccatatttaacaacgccaacatgtaatttaggcagaggcattttcgagccagtaataagagaatataaagtaccgacaaaaggtaaagtaattctgtccagacgacgacaataaacaacatgttcagctaatgcagaacgcgcctgtttatcaacaatagataagtcctgaacaagaaaaataatatcccatcctaatttacgagcatgtagaaaccaatcaataatcggctgtctttccttatcattccaagaacgggtattaaaccaagtaccgcactcatcgagaacaagcaagccgtttttattttcatcgtaggaatcattaccgcgcccaatagcaagcaaatcagatatagaaggcttatccggtattctaagaacgcgaggcgttttagcgaacctcccgacttgcgggaggttttgaagccttaaatcaagattagttgctattttgcacccagctacaattttatcctgaatcttaccaacgctaacgagcgtctttccagagcctaatttgccagttacaaaataaacagccatattatttatcccaatccaaataagaaacgattttttgtttaacgtcaaaaatgaaaatagcagcctttacagagagaataacataaaaacagggaagcgcattagacgggagaattaactgaacaccctgaacaaagtcagagggtaattgagcgctaatatcagagagataacccacaagaattgagttaagcccaataataagagcaagaaacaatgaaatagcaatagctatcttaccgaagccctttttaagaaaagtaagcagatagccgaacaaagttaccagaaggaaaccgaggaaacgcaataataacggaatacccaaaagaactggcatgattaagactccttattacgcagtatgttagcaaacgtagaaaatacatacataaaggtggcaacatataaaagaaacgcaaagacaccacggaataagtttattttgtcacaatcaatagaaaattcatatggtttaccagcgctaaagacaaaagggcgacattcaaccgattgagggagggaaggtaaatattgacggaaattattcattaaaggtgaattatcaccgtcaccgacttgagccatttgggaattagagccagcaaaatcaccagtagcaccattaccattagcaaggccggaaacgtcaccaatgaaaccatcgatagcagcaccgtaatcagtagcgacagaatcaagtttgcctttagcgtcagactgtagcgcgttttcatcggcattttcggtcatagcccccttattagcgtttgccatcttttcataatcaaaatcaccggaaccagagccaccaccggaaccgcctccctcagagccgccaccctcagaaccgccaccctcagagccaccaccctcagagccgccaccagaaccaccaccagagccgccgccagcattgacaggaggttgaggcaggtcagacgattggccttgatattcacaaacgaatggatcctcattaaagccagaatggaaagcgcagtctctgaatttaccgttccagtaagcgtcatacatggcttttgatgatacaggagtgtactggtaataagttttaacggggtcagtgccttgagtaacagtgcccgtataaacagttaatgccccctgcctatttcggaacctattattctgaaacatgaaagtattaagaggctgagactcctcaagagaaggattaggattagcggggttttgctcagtaccaggcggataagtgccgtcgagagggttgatataagtatagcccggaataggtgtatcaccgtactcaggaggtttagtaccgccaccctcagaaccgccaccctcagaaccgccaccctcagagccaccaccctcattttcagggatagcaagcccaataggaacccatgtaccgtaacactgagtttcgtcaccagtacaaactacaacgcctgtagcattccacagacaaccctcatagttagcgtaacgatctaaagttttgtcgtctttccagacgttagtaaatgaattttctgtatggggttttgctaaacaactttcaacagtttcagcggagtgagaatagaaaggaacaactaaaggaattgcgaataataattttttcacgttgaaaatctccaaaaaaaaaggctccaaaaggagcctttaattgtatcggtttatcagcttgctttcgaggtgaatttcttaaacagcttgataccgatagttgcgccgacaatgacaacaaccatcgcccacgcataaccgatatattcggtcgctgaggcttgcagggagttaaaggccgcttttgcgggatcgtcaccctcagcagcgaaagacagcatcggaacgagggtagcaacggctacagaggctttgaggactaaagactttttcatgaggaagtttccattaaacgggtaaaatacgtaatgccactacgaaggcaccaacctaaaacgaaagaggcgaaagaatacactaaaacactcatctttgacccccagcgattataccaagcgcgaaacaaagtacaacggagatttgtatcatcgcctgataaattgtgtcgaaatccgcgacctgctccatgttacttagccggaacgaggcgcagacggtcaatcataagggaaccgaactgaccaactttgaaagaggacagatgaacggtgtacagaccaggcgcataggctggctgaccttcatcaagagtaatcttgacaagaaccggatattcattacccaaatcaacgtaacaaagctgctcattcagtgaataaggcttgccctgacgagaaacaccagaacgagtagtaaattgggcttgagatggtttaatttcaactttaatcattgtgaattaccttatgcgattttaagaactggctcattataccagtcaggacgttgggaagaaaaatctacgttaataaaacgaactaacggaacaacattattacaggtagaaagattcatcagttgagatttaggaataccacattcaactaatgcagatacataacgccaaaaggaattacgaggcatagtaagagcaacactatcataaccctcgtttaccagacgacgataaaaaccaaaatagcgagaggcttttgcaaaagaagttttgccagagggggtaatagtaaaatgtttagactggatagcgtccaatactgcggaatcgtcataaatattcattgaatccccctcaaatgctttaaacagttcagaaaacgagaatgaccataaatcaaaaatcaggtctttaccctgactattatagtcagaagcaaagcggattgcatcaaaaagattaagaggaagcccgaaagacttcaaatatcgcgttttaattcgagcttcaaagcgaaccagaccggaagcaaactccaacaggtcaggattagagagtacctttaattgctccttttgataagaggtcatttttgcggatggcttagagcttaattgctgaatctggtgctgtagctcaacatgttttaaatatgcaactaaagtacggtgtctggaagtttcattccatgtaacagttgattcccaattctgcgaacgagtagatttagtttgaccattagatacatttcgcaaatggtcaataacctgtttagctatattttcatttggggcgcgagctgaaaaggtggcatcaattctactaatagtagtagcgtt

SEQ ID NO: 4 - DNA, 4500 bp, Artificial Sequence
  synthetic sequence of pDSP7
ttcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcttttcaaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagtttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactatagggagaccacaacggtttccctctagacgttacagcgactactgaaacgtaagtcgttcttttatgggaggacataatgtccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgagtcaacctaaaactggatcaggcggacgtcgttgattgctccaccagcgtctgcggcgagcttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgttatggctccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgagtcaacctaaaactggatcaggcggacgtcgttgattgctccaccagcgtctgcggcgagcttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgttaaggccctgcggtgtacccgtgtgtatggatccggctgctaacaaagcccgaaaggaagctgagttggctgctgccaccgctgagcaataactagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgaa

SEQ ID NO: 5 - DNA, 4500 bp, Artificial Sequence
  synthetic sequence of pDSP7K
ttcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcttttcaaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagtttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactatagggagaccacaacggtttccctctagacgttacagcgactactgaaacgtaagtcgttcttttatgggaggacataatgtccaaaaccatcgttctttcggtcggtaccgctactcgcactctgactgagatccagtccaccgcagaccgtcagatcttcgaagagaaggtcgggcctctggtgggtcggctgcgcctcacggcttcgctccgtcaaaacggagccaagaccgcgtatcgagtcaacctaaaactggatcaggcggacgtcgttgattgctccaccagcgtctgcggcgagcttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgttatggctccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgagtcaacctaaaactggatcaggcggacgtcgttgattgctccaccagcgtctgcggcgagcttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgttaaggccctgcggtgtacccgtgtgtatggatccggctgctaacaaagcccgaaaggaagctgagttggctgctgccaccgctgagcaataactagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgaa

SEQ ID NO: 6 - DNA, 4564 bp, Artificial Sequence
  synthetic sequence of p6H
ggggaattgtgagcggataacaattcccctgtagaaataattttgtttaactttaataaggagatataccatggcttctaactttactcagttcgttctcgtcgacaatggcggaactggcgacgtgactgtcgccccaagcaacttcgctaacggggtcgctgaatggatcagctctaactcgcgttcacaggcttacaaagtaacctgtagcgttcgtcagagctctgcgcagaatcgcaaatacaccatcaaagtcgaggtgcctaaagtggcaacccagactgttggtggtgtagagcttcctgtagccgcatggcgttcgtacttaaatatggaactaaccattccaattttcgctacgaattccgactgcgagcttattgttaaggcaatgcaaggtctcctaaaagatggaaacccgattccctcagcaatcgcagcaaactccggcctctacggcaactttactcagttcgttctcgtcgacaatggcggtacccatcaccatcaccatcacggaactggcgacgtgactgtcgccccaagcaacttcgctaacggggtcgctgaatggatcagctctaactcgcgttcacaggcttacaaagtaacctgtagcgttcgtcagagctctgcgcagaatcgcaaatacaccatcaaagtcgaggtgcctaaagtggcaacccagactgttggtggtgtagagcttcctgtagccgcatggcgttcgtacttaaatatggaactaaccattccaattttcgctacgaattccgactgcgagcttattgttaaggcaatgcaaggtctcctaaaagatggaaacccgattccctcagcaatcgcagcaaactccggcatctactaatagacgccgggttaattaattaaggatccccgggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatagtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactata

SEQ ID NO: 7 - protein, 10 aa, Artificial Sequence
  synthetic sequence of mAb--10, containing perfect match to natural G protein epitope.
GTNQTAENPI

SEQ ID NO: 8 - protein, 10 aa, Nipah Virus
  BINDING (3)..(9): G protein epitope.
DSNQTAENPV

SEQ ID NO: 9 - protein, 10 aa, Artificial Sequence
  synthetic sequence of mAb--26, containing perfect match to natural G protein.
DSGWEGVYRQ

SEQ ID NO: 10 - protein, 10 aa, Nipah Virus
  BINDING (4)..(8): G protein epitope.
EVCWEGVYND

SEQ ID NO: 11 - protein, 10 aa, Artificial Sequence
  synthetic sequence of mAb--55, containing perfect match to natural G protein.
GDDTNAQRAF

SEQ ID NO: 12 - protein, 10 aa, Nipah Virus
  BINDING (3)..(7)
SEDTNAQKTI

SEQ ID NO: 13 - protein, 10 aa, Artificial Sequence
  synthetic sequence of mAb--101, containing perfect match to natural G protein.
VRLQKTSKGY

SEQ ID NO: 14 - protein, 10 aa, Artificial Sequence
  synthetic sequence of mAb--101, containing perfect match to natural G protein.
TNICLQKAGW

SEQ ID NO: 15 - protein, 10 aa, Artificial Sequence
  synthetic sequence of mAb--101, containing perfect match to natural G proteiin.
GKQALPNNIR

SEQ ID NO: 16 - protein, 10 aa, Artificial Sequence
  synthetic sequence of mAb--102, containing perfect match to natural G protein.
GAIVGLPNQG

SEQ ID NO: 17 - protein, 24 aa, Nipah Virus
  BINDING (5)..(15): relevant residues for sequences 13-16.
VSNLVGLPDNICLQKTSNQILKPK

SEQ ID NO: 18 - DNA, 54 bp, Artificial Sequence
  synthetic sequence of wild-type PP7 coat protein N-terminal sequence.
atggccaaaaccatcgttctttcggtcggcgaggctactcgcactctgactgag

SEQ ID NO: 19 - protein, 18 aa, Artificial Sequence
  synthetic sequence of wild-type PP7 coat protein N-terminial sequence.
MAKTIVLSVGEATRTLTE

SEQ ID NO: 20 - DNA, 54 bp, Artificial Sequence
  synthetic sequence of pP7K mutant coat protein sequence.
atggccaaaaccatcgttctttcggtcggtaccgctactcgcactctgactgag

SEQ ID NO: 21 - protein, 18 aa, Artificial Sequence
  synthetic sequence of pP7K mutant coat protein sequence.
MAKTIVLSVGTATRTLTE

SEQ ID NO: 22 - DNA, 48 bp, Artificial Sequence
  --primer for insertion of random peptide sequence between residues 11 and 12, of NNY hexamer.
gcgggtaccnnynnynnynnynnynnygctactcgcactctgactgag

SEQ ID NO: 23 - DNA, 51 bp, Artificial Sequence
  synthetic sequence--product with NNY-hexamer insertion.
gcgggtaccnnynnynnynnynnynnygaggctactcgcactctgactgag

SEQ ID NO: 24 - DNA, 57 bp, Artificial Sequence
  synthetic sequence--product of insertion of NNY-octamer.
gcgggtaccnnynnynnynnynnynnynnynnygaggctactcgcactctgactgag

SEQ ID NO: 25 - DNA, 63 bp, Artificial Sequence
  synthetic sequence--product of NNY--decimer insertion.
gcgggtaccnnynnynnynnynnynnynnynnynnynnygaggctactcgcactctgactgag

SEQ ID NO: 26 - DNA, 57 bp, Artificial Sequence
  synthetic sequence--primers constructed for specific peptide insertions.
gccggtaccgattataaagatgatgatgataaagaggctactcgcactctgactgag

SEQ ID NO: 27 - DNA, 63 bp, Artificial Sequence
  synthetic sequence--primer constructed for specific peptide insertion.
gccggtaccgtgcatgcgagcttttttgatatcggcggcgaggctactcgcactctgactgag

SEQ ID NO: 28 - DNA, 60 bp, Artificial Sequence
  synthetic sequence--primer constructed for specific peptide insertion.
gccggtaccnnsnnsgcgagcttttttgatnnsnnsgaggctactcgcactctgactgag

SEQ ID NO: 29 - DNA, 63 bp, Artificial Sequence
  synthetic sequence--primer constructed for specific peptide insertion.
gccggtaccatccagcgcggcccgggccgcgcgtttgtggaggctactcgcactctgactgag

SEQ ID NO: 30 - DNA, 78 bp, Artificial Sequence
  synthetic sequence--primer constructed for specific peptide insertion.
gccggtacccagctgtataaaacctgcaaacaggcgggcacctgcccgccggatgaggctactcgcactctgactgag

SEQ ID NO: 31 - DNA, 57 bp, Artificial Sequence
  synthetic sequence--primer constructed for specific peptide insertion.
gccggtacccagctgtataaaacctgcaaacaggaggctactcgcactctgactgag

SEQ ID NO: 32 - protein, 17 aa, Artificial Sequence
  synthetic sequence--MS2 coat sequence in region of AB-loop insertion site for pDSP1.
QFVLVDNGGTGDVTVAP

SEQ ID NO: 33 - DNA, 50 bp, Artificial Sequence
  synthetic sequence--MS2 coat sequence in region of AB-loop insertion site for pDSP1.
cagttcgttctcgtcgacaatggcggtaccggcgacgtgactgtcgccca

SEQ ID NO: 34 - protein, 22 aa, Artificial Sequence
  synthetic sequence--a random 7-mer library in pDSP1, with random sequence in the so-called 13/16 mode. The gene product is shown here.
QFVLVDNGXXXXXXXGDVTVAP

SEQ ID NO: 35 - DNA, 66 bp, Artificial Sequence
  synthetic sequence--a random 7-mer library in pDSP1, with the random sequence inserted in the so-called 13/16 mode.
cagttcgttctcgtcgacaatggcnnsnnsnnsnnsnnsnnsnnsggcgacgtgactgtcgcccca

SEQ ID NO: 36 - DNA, 57 bp, Artificial Sequence
  synthetic sequence--5′-PCR primer used with a 3′ primer to generate a fragment of the coat protein as described in the specification.
cgcgtcgacaatggcnnsnnsnnsnnsnnsnnsnnsggcgacgtgactgtcgcccca